(19) United States
(12) Patent Application Publication
(10) Pub. No.: US 2021/0269828 A1
(43) Pub. Date: Sep. 2, 2021

(54) VECTORS FOR GENE DELIVERY THAT PERSIST WITHIN CELLS

(71) Applicant: ASKLEPIOS BIOPHARMACEUTICAL, INC., Research Triangle Park, NC (US)

(72) Inventor: Richard Jude SAMULSKI, Hillsborough, NC (US)

(73) Assignee: ASKLEPIOS BIOPHARMACEUTICAL, INC., Research Triangle Park, NC (US)

(21) Appl. No.: 17/253,929

(22) PCT Filed: Jun. 21, 2019
(86) PCT No.: PCT/US2019/038515

§ 371 Date: Dec. 18, 2020

Related U.S. Application Data
(60) Provisional application No. 62/688,982, filed on Jun. 22, 2018.
(60) Provisional application No. 62/689,453, filed on Jun. 25, 2018.
(60) Provisional application No. 62/697,750, filed on Jul. 13, 2018.
(60) Provisional application No. 62/717,310, filed on Aug. 10, 2018.

(51) Int. Cl. C12N 15/86

(52) U.S. Cl. CPC C12N 15/86; A61K 48/00; C12N 2750/14143

(57) ABSTRACT

Disclosed herein are vectors for delivery of nucleic acid sequence into a target cell. The vectors are non-viral DNA constructs. The vectors have at least one DD-ITR, and complementary copies of the nucleic acid sequence operatively linked to regulatory elements that promote expression. The construct has covalently closed ends having a hairpin structure, and persists within the recipient cells as they divide. Delivery of the vector to the target cell results in sustained expression of the nucleic acid sequences in the target cell. Also disclosed are DNA vector constructs having at least one synthetic ITR, wherein the DNA construct forms linear DNA with hairpin covalently closed ends. Methods of generating the constructs and introducing target cells to thereby promote sustained expression of the nucleic acid sequences contained therein, are also disclosed. Further disclosed are cells and populations thereof, which contain the vectors.

### CROSS-REFERENCE TO RELATED APPLICATION

This Application is a 371 National Phase Entry of International Patent Application No. PCT/US2019/038515 filed on Jun. 21, 2019, which claims benefit under 35 U.S.C. § 119(e) of the U.S. Provisional Application No. 62/688,982 filed Jun. 22, 2018; U.S. Provisional Application No. 62/689,453 filed Jun. 25, 2018; U.S. Provisional Application No. 62/697,750 filed Jul. 13, 2018; and U.S. Provisional Application No. 62/717,310 filed Aug. 10, 2018, the contents of each of which are incorporated herein by reference in their entireties.

### FIELD OF THE INVENTION

The present invention relates to the field of vectors and methods for the cell-free production, introduction and expression of nucleic acid sequences into mammalian cells, such as for gene therapy.

### SEQUENCE LISTING

The instant application contains a Sequence Listing which has been submitted electronically in ASCII format and is hereby incorporated by reference in its entirety. Said ASCII copy, created on Dec. 17, 2020, is named 046192-092620WOPT_SL.txt and is 37,123 bytes in size.

### BACKGROUND OF THE INVENTION

It is desirable to introduce the exogenous DNA in a manner such that it provides for long-term expression of the protein encoded by the exogenous DNA. Viral based protocols have been developed, in which a viral vector is employed to introduce exogenous DNA into a cell that can subsequently integrate the introduced DNA into the target cell's genome or remain episomally. Viral based vectors finding use include retroviral vectors, e.g., Moloney murine leukemia viral based vectors, adenovirus derived vectors, adeno-associated virus (AAV) derived vectors, HSV derived vectors, sindbis derived vectors, etc. A great deal of interest has focused on the use of AAV vectors. There are 12 serotypes that are most common. A typical method for production of AAV vectors is using cells such as insect cells, e.g., SF-9, or mammalian cells, that express AAV rep (rep is required to initiate replication of the AAV), and cap (cap is needed for the production of AAV) genes for the formation of virions, and introducing into these cells a plasmid with AAV inverted terminal repeats (ITRs) flanking a cassette containing a promoter operably linked to a gene of interest. An additional plasmid with viral helper genes, for example derived from adenovirus or herpes simplex virus, is also needed to replicate and obtain the packaged AAV capsids used for transduction. However, it is difficult to completely eliminate the helper virus, e.g., adenovirus, from the AAV products. In addition, the use of viral proteins can elicit an unwanted immune response.

Therefore, there is continued interest in the development of additional methods of production of vectors, and for transfecting cells with exogenous nucleic acids to provide for persistent, long-term expression of an encoded protein, or nucleic acid, e.g. a therapeutic protein or nucleic acid.

### SUMMARY OF THE INVENTION

Aspects of the invention relate to an in vitro cell-free environment to generate vectors for gene delivery without the need of a helper virus. This cell-free synthesis is easier and safer to use for vector production. However, one of skill will understand that while the cell-free methods described herein may be preferred for therapeutic use, the vectors described herein can also be produced within cells, e.g. bacterial, mammalian, or insect cells.

Aspects of the invention also relate to a methods and vector constructs for introducing a nucleic acid construct into a target cell for sustained expression. In one aspect, the method comprises administering to the target cell a covalently closed linear non-viral DNA construct comprising at least one DD-ITR comprising i) an inverted terminal repeat having an A, A′, B, B′, C, C′ and D region; ii) a D′ region; iii) wherein the D and D′ region are complementary palindromic sequences, and wherein D and D′ are positioned adjacent the A and A′ region. In one embodiment the regions correspond to a parvoviral ITR, e.g. the AAV A, A′, B, B′, C, C′ and D regions. In one embodiment the ITR corresponds to a parvoviral ITR that is a wild type ITR, a mutant ITR, or a synthetic ITR. The DNA vector construct further comprises complementary strands of the nucleic acid construct comprising a cassette containing a promoter operably linked to a predetermined DNA sequence that can anneal into expressible dsDNA, wherein the DNA construct forms linear DNA with covalently closed hairpin ends, and wherein the DNA construct can express the predetermined DNA sequence in the target cell. In one embodiment, the covalently closed non-viral linear DNA construct comprises at least two DD-ITRs, e.g. as illustrated in FIG. 1.

Aspects of the invention further relate to a DNA vector for delivery of a predetermined nucleic acid sequence (e.g. heterologous gene) into a target cell for sustained expression. The DNA vector is a covalently closed non-viral linear DNA vector for delivery of predetermined nucleic acid into a target cell for sustained expression that comprises a) at least one DD-ITR comprising: i) an inverted terminal repeat having an A, A′, B, B′, C, C′ and D region; ii) a D′ region; iii) wherein the D and D′ region are complementary palindromic sequences, and wherein D and D′ are positioned adjacent the A and A′ region; b) complementary strands of the nucleic acid construct comprising a predetermined DNA sequence that can anneal into expressible dsDNA; c) wherein the DNA vector construct forms linear DNA with covalently closed hairpin ends; and d) wherein the DNA vector construct can express the predetermined DNA sequence in the target cell. In one embodiment the regions correspond to a parvoviral ITR, e.g. the AAV A, A′, B, B′, C, C′ and D regions. In one embodiment the ITR correspond to a wild type ITR, a mutant ITR, or a synthetic ITR. The DNA vector construct further comprises complementary strands of the nucleic acid construct comprising a cassette containing a promoter operably linked to a predetermined DNA sequence that can anneal into expressible dsDNA, wherein the DNA construct forms linear DNA with covalently closed hairpin ends, and wherein the DNA construct can express the predetermined DNA sequence in the target cell. In one embodiment, the vector comprises at least two DD-ITRs that flank the nucleic acid in the context of covalently closed non-viral linear DNA that expresses a predetermined heterologous transgene, e.g. a protein or nucleic acid. One embodiment of this vector is illustrated in the bottom of FIG. 1. The vector is substantially complimentary DNA having covalently closed hairpin ends. In one embodiment, the D regions are about 20 nt in length. The D and D′ region may have substitution, insertion, and/or deletions that retains at least 5 nucleic acids of the region. In one embodiment, the retained nucleic acids may comprise the nicking site and/or junction of the A and A′ region and the D and D′ regions.

Aspects of the invention further relate to a DNA vector for delivery of a predetermined nucleic acid sequence into a target cell for sustained expression. The vector comprises two DD-ITRs each comprising i) an inverted terminal repeat having an A, A′, B, B′, C, C′ and D region;

ii) a D′ region; iii) wherein the D and D′ region are complementary palindromic sequences and are positioned adjacent the A and A′ region. The vector further comprises the predetermined nucleic acid sequence. The two DD-ITRs flank the nucleic acid in the context of covalently closed non-viral DNA, e.g. mini-circle DNA, wherein a majority of the vector is non-bacterial sequences.

Aspects of the invention relate to a method for introducing a nucleic acid into a target cell for sustained expression comprising administering to the target cell a covalently closed non-viral DNA construct. The DNA construct comprises at least one ITR sequence selected from the group consisting of the ITR's shown in FIG. 5, complementary strands of the nucleic acid construct, wherein the nucleic acid construct comprises a predetermined DNA sequence, wherein the complementary strands can anneal into expressible dsDNA; and wherein the DNA construct forms linear DNA with hairpin covalently closed ends.

Further aspects of the invention relate to use of the covalently closed non-viral linear DNA (sometimes closed linear DNA) to produce recombinant viral particles comprising construct DNA. This involves adding the closed linear DNA construct comprising at least two DD-ITRs (e.g., see FIG. 1) into a host cell along with vectors or plasmids capable of (1) expressing AAV Rep proteins necessary to nick at least one of the DD-ITRs, and (2) expressing parvovirus viral capsid proteins, e.g., a dependovirus such as AAV, necessary to form a virion (viral particle) that can encapsidate the ITRs and the intervening DNA sequence. In the host cell the DD-ITRs resolve and replication is initiated from the self-primed ITR in the presence of Rep to produce vector genomes that can be packaged. Preferably, the intervening DNA sequence contains a transgene sequence such as a therapeutic gene sequence. The particular Rep protein that nicks, e.g., an AAV ITR is typically from the same serotype (for example, AAV2 ITR with AAV2Rep78) although any serotypes can be used as long as they retain at least 25% . . . 35% . . . 50% . . . 65% . . . 75% . . . 85% . . . 90% . . . 95% . . . 98% . . . 100% or any intervening percent or greater percent nicking efficacy as compared to the situation when the AAV ITR and the AAV Rep protein are from the same serotype. One can use this method to prepare large amounts of viral particles containing the ITRs and their intervening DNA sequence, e.g. transgene. Using the closed linear vector containing the DD-ITR is more efficient, i.e. it provides a higher yield of packaged genomes, infectious viral particles as compared to a closed linear vector not having the Double D, and the viral particles do not have any contaminating plasmid DNA.

With this method one can produce any known AAV particles such as those containing synthetic ITRs, or self-complementary dimeric AAV sequences (sc dimer) etc. The skilled artisan knows that the typical AAV particle packages a single stranded DNA genome and cannot package more than about 5,000 nts and thus care is taken in designing the initial non-viral closed linear DNA construct to be a packagable size. In one embodiment, when packaged self-complementary is desired the initial non-viral closed linear DNA construct used as a rAAV vector template, for example can have a Rep nick defective ITR or a hairpin sequence. See for example, U.S. Pat. Nos. 7,465,583, 7,790,154, 8,361,457, 8,784,799, the contents of which are incorporated herein by reference in their entirety. In one embodiment, the template is approximately ½ size template and one of the DD-ITRs is a Rep-nick incompetent ITR. In one embodiment, the nick defective ITR, or hairpin sequence, is placed between to two Rep-nick competent DD-ITRs and the nick defective ITR, or hairpin sequence is further in between the sense and anti-sense sequences of the transgene. In either case, after replication of the template the sense and anti-sense sequences are on a single strand of DNA in between ITRs and this vector genome can be packaged.

The host cell can already be expressing the Rep and viral capsid proteins or one can co-transfect the cells at the same or about the same time by standard means. Any of the known cell-types used in the art can be used in this process. For example, one can use mammalian cells, or insect cells such as baculovirus.

Pharmaceutical compositions comprising the vectors described herein are also provided.

In embodiments of the methods and vectors described herein, the ITR sequence is flanked on either side by complementary sequences D and D′.

In embodiments of the methods and vectors described herein, the D regions contain a nicking site.

In embodiments of the methods and vectors described herein, the D regions are at least 5 nucleotides in length.

In embodiments of the methods and vectors described herein, the D regions are about 20 nt in length.

In embodiments of the methods and vectors described herein, the D region corresponds to a parvovirus D region of a parvovirus ITR.

In embodiments of the methods and vectors described herein, the parvovirus is a dependovirus.

In embodiments of the methods and vectors described herein, the dependovirus is AAV.

In embodiments of the methods and vectors described herein, the predetermined DNA/nucleic acid sequence is operably linked to a promoter.

In embodiments of the methods and vectors described herein, the ITR is acting as a promoter.

In embodiments of the methods and vectors described herein, the promoter is separate from the ITR.

In embodiments of the methods and vectors described herein, the DD-ITR drives expression of the predetermined DNA/nucleic acid sequence.

In embodiments of the methods and vectors described herein, the D and D′ region has a substitution, insertion, and/or deletion that retains at least 5 nucleic acids of the region.

In embodiments of the methods and vectors described herein, the retained nucleic acids comprise the nicking site and/or junction of the A and A′ region and the D and D′ regions.

In embodiments of the methods and vectors described herein, the predetermined DNA sequence/nucleic acid sequence encodes a protein, a protein fragment, a peptide, or a functional RNA.

In embodiments of the methods and vectors described herein, the functional RNA is selected from the group consisting of micro RNA, RNAi, shRNA, and guide RNA for Crisper Cas 9 recombination.

In embodiments of the methods and vectors described herein, there are at least 2 nucleotides between the D and D′ region and the predetermined DNA sequence/nucleic acid as spacers.

In embodiments of the methods and vectors described herein, there are at least 2 nucleotides between the D and D′ region and the promoter as spacers.

In embodiments of the methods and vectors described herein, the spacers are at least 5 nucleotides.

In embodiments of the methods and vectors described herein, the spacers are at least 20 nucleotides.

In embodiments of the methods and vectors described herein, the spacers are at least 25 nucleotides.

In embodiments of the methods and vectors described herein, at least one DD-ITR is generated from an AAV ITR, a parvovirus ITR, or a synthetic ITR.

In embodiments of the methods and vectors described herein, the DNA construct comprises two or more DD-ITRs.

In embodiments of the methods and vectors described herein, the D regions are from different stereotypes than the ITR.

In embodiments of the methods and vectors described herein, multiple DD-ITRs are present and one or more of the DD-ITRs are derived from a different viral serotype than the other(s).

In embodiments of the methods and vectors described herein, one DD-ITR is derived from an AAV2 ITR, and a second DD-ITR is derived from an AAV5 ITR.

In embodiments of the methods and vectors described herein, there is a deletion, substitution and/or insertion in the B and B′ or C and C′ region.

In embodiments of the methods and vectors described herein, there is a deletion, substitution and/or insertion in the A and A′ region.

In embodiments of the methods and vectors described herein, the DNA construct further comprises a partial protelomerase binding site and wherein the covalently closed ends are formed by protelomerase enzyme activity in vitro.

In embodiments of the methods and vectors described herein, the DNA construct persists within the target cell and results in sustained expression of the predetermined sequence (e.g., as a result of either replication or recombination).

In embodiments of the methods and vectors described herein, the DNA construct can be converted into a concatemeric structure in the cell.

In embodiments of the methods and vectors described herein, the sustained expression of the predetermined DNA sequence in the target cell is for a period of time at least at least 2-5 weeks, at least 1-12 months, at least 1-10 years.

In embodiments of the methods and vectors described herein, the concatemeric structure persists in the target cell and results in sustained expression of the predetermined sequence (e.g., as a result of either replication or recombination).

In embodiments of the methods and vectors described herein, the concatemeric structure persists in the target cell extra-chromosomally.

In embodiments of the methods and vectors described herein, the concatemeric structure integrates into the target cell chromosome.

In embodiments of the methods and vectors described herein, the nucleic acid is a therapeutic nucleic acid.

In embodiments of the methods and vectors described herein, the target cell is in vitro.

In embodiments of the methods and vectors described herein, the target cell is in vivo.

In embodiments of the methods and vectors described herein, the construct is administered to the target cell ex vivo.

In embodiments of the methods and vectors described herein, the target cell is a genetically deficient cell and/or a diseased cell.

In embodiments of the methods and vectors described herein, the target cell is selected from the group consisting of a neural cell, lung cell, retinal cell, epithelial cell, smooth muscle cell, skeletal muscle cell cardiac muscle cell, pancreatic cell, hepatic cell, kidney cell, myocardial cell, bone cell, spleen cell, keratinocyte, fibroblast, endothelial cell, prostate cell, germ cell, progenitor cell, stem cell, cancer cell, and tumor cell.

Another aspect of the invention relates to a cell or population thereof, produced by the methods described herein.

### Definitions

The singular forms “a” and “an” are intended to include the plural forms as well, unless the context clearly indicates otherwise.

The term “about,” as used herein when referring to a measurable value such as an amount of the length of a polynucleotide or polypeptide sequence, dose, time, temperature, and the like, is meant to encompass variations of 20%, 10%, 5%, 1%, 0.5%, or even 0.1% of the specified amount.

As used herein, “and/or” refers to and encompasses any and all possible combinations of one or more of the associated listed items, as well as the lack of combinations when interpreted in the alternative (“or”).

The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panlcukopenia virus, feline parvovirus, goose parvovirus, HI parvovirus, muscovy duck parvovirus, snake parvovirus, and B19 virus. Other autonomous parvoviruses are known to those skilled in the art. See, e.g., FIELDS et al. VIROLOGY, volume 2, chapter 69 (4th ed., Lippincott-Raven Publishers).

The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. See, e.g., FIGS. 8-19; FIELDS et al. VIROLOGY, volume 2, chapter 69 (4th ed., Lippincott-Raven Publishers).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, and any other AAV now known or later discovered. See, e.g., FIELDS et al. VIROLOGY, volume 2, chapter 69 (4th ed., Lippincott-Raven Publishers). A number of relatively new AAV serotypes and clades have been identified (See, e.g., Gao et al. (2004) J. Virol. 78:6381; Moris et al. (2004) Virol. 33-:375).

The term “inverted terminal repeat” or “ITR” includes any viral terminal repeat or synthetic sequence that forms a hairpin structure and functions as an inverted terminal repeat (i.e., mediates the desired functions such as replication, integration and/or provirus rescue, and the like). The ITR can be an AAV ITR or a non-AAV ITR. For example, a non-AAV ITR sequence such as those of other parvoviruses (e.g., canine parvovirus, bovine parvovirus, mouse parvovirus, porcine parvovirus, human parvovirus B-19) or the SV40 hairpin that serves as the origin of SV40 replication can be used as an ITR, which can further be modified by truncation, substitution, deletion, insertion and/or addition. Further, the ITR can be partially or completely synthetic, such as shown in FIG. 5, and described in U.S. Pat. No. 9,169,494, the contents of which are incorporated herein by reference in their entirety. Typically, the ITR is 145 nucleotides. The terminal 125 nucleotides form a palindromic double stranded T-shaped hairpin structure. In the structure the A-A′ palindrome forms the stem, and the two smaller palindromes B-B′ and C-C′ form the cross-arms of the T. The other 20 nucleotides in the D sequence remain single stranded. In the context of an AAV genome, there would be two ITR's, one at each end of the genome, and there would be a single D sequence, D or D′, for each ITR.

A DD-ITR has the same sequence the ITR from which it is derived, but includes a second D sequence adjacent the A sequence, so there are D and D′. The D and D′ can anneal (e.g., as described in U.S. Pat. No. 5,478,745, the contents of which are incorporated herein by reference). Each D is typically about 20 nt in length, but can be as small as 5 nucleotides. Shorter D regions preserve the A-D junction (e.g., are generated by deletions at the 3′ end that preserve the A-D junction). Preferably the D region retains the nicking site and/or the A-D junction. The DD-ITR is typically about 165 nucleotides. The DD-ITR has the ability to provide information in cis for replication of the DNA construct. Thus, a DD-ITR has an inverted palindromic sequence with flanking D and D′ elements, e.g. a (+) strand 5′ to 3′ sequence of 5′-DABB′CC′A′D′-3′ and a (−) strand complimentary to the (+) strand that has a 5′ to 3′ sequence of 5′-DACC′BB′A′D′-3′ that can form a Holiday structure, e.g. as illustrated in FIG. 1. In certain embodiments, the DD-ITR may have deletions in its components (e.g. A-C), while still retaining the D and D′ element. In certain embodiments, the ITR comprises deletions while still retaining the ability to form a Holliday structure and retaining two copies of the D element (D and D′). The DD-ITR may be generated from a native AAV ITR or from a synthetic ITR. In certain embodiments, the deletion is in the B region element. In certain embodiments, the deletion is in the C region element. In certain embodiments, a deletion within both the B and C element of the ITR. In one embodiment, the entire B and/or C element is deleted, and e.g. replaced with a single hairpin element.

An “AAV inverted terminal repeat” or “AAV ITR” may be from any AAV, including but not limited to serotypes 1, 2, 3a, 3b, 4, 5, 6, 7, 8, 9, 10, 11, or 13, snake AAV, avian AAV, bovine AAV, canine AAV, equine AAV, ovine AAV, goat AAV, shrimp AAV, or any other AAV now known or later discovered (see, e.g., Table 1). An AAV ITR need not have the native terminal repeat sequence (e.g., a native AAV ITR sequence may be altered by insertion, deletion, truncation and/or missense mutations), as long as the terminal repeat mediates the desired functions, e.g., replication, or, integration.

As used herein, the term “synthetic ITR” refers to a non-naturally occurring ITR that differs in nucleotide sequence from wild-type ITRs, e.g., the AAV serotype 2 ITR (ITR2) sequence due to one or more deletions, additions, substitutions, or any combination thereof. The difference between the synthetic and wild-type ITR (e.g., ITR2) sequences may be as little as a single nucleotide change, e.g., a change in 1, 2, 3, 4, 5, 6, 7, 8, 9, 10, 15, 20, 25, 30, 35, 40, 45, 50, 60, 60, 70, 80, 90, or 100 or more nucleotides or any range therein. In some embodiments, the difference between, the synthetic and wild-type ITR (e.g., ITR2) sequences may be no more than about 100, 90, 80, 70, 60, 50, 45, 40, 35, 30, 25, 20, 15, 10, 9, 8, 7, 6, 5, 4, 3, 2, or 1 nucleotide or any range therein.

In some embodiments of the invention, the D regions are derived from different serotypes than the A, B and C regions.

The terms “nucleotide sequence” and “nucleic acid”, DNA sequence, are used interchangeably herein and refer to a sequence that is to be delivered into a target cell. Generally, the nucleic acid comprises an open reading frame that encodes a polypeptide or nontranslated RNA of interest (e.g., for delivery to a cell or subject). The nucleic acid sequence may further comprise regulatory sequences, the combination of which may be referred to as a transgene or expression construct. Preferably the nucleic acid is heterologous, that is not naturally occurring in conjunction with the ITR (e.g. not naturally occurring in a virus from which an ITR is derived). Such a nucleic acid is referred to as heterologous.

A “promoter” is a nucleotide sequence which initiates and regulates transcription of a polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

“Operably linked” refers to an arrangement of elements wherein the components so described are configured so as to perform their usual function. Thus, a given promoter operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence. Thus, the term “operably linked” is intended to encompass any spacing or orientation of the promoter element and the DNA sequence of interest which allows for initiation of transcription of the DNA sequence of interest upon recognition of the promoter element by a transcription complex.

A protelomerase target sequence is any DNA sequence whose presence in a DNA template allows for its conversion into a closed linear DNA by the enzymatic activity of protelomerase. In other words, the protelomerase target sequence is required for the cleavage and religation of double stranded DNA by protelomerase to form covalently closed linear DNA.

Typically, a protelomerase target sequence comprises any perfect palindromic sequence i.e any double-stranded DNA sequence having two-fold rotational symmetry, also described herein as a perfect inverted repeat. As shown in U.S. Pat. No. 9,109,250, the contents of which are incorporated by reference in their entirety, the protelomerase target sequences from various mesophilic bacteriophages, and a bacterial plasmid all share the common feature of comprising a perfect inverted repeat. The length of the perfect inverted repeat differs depending on the specific organism. In Borrelia burgdorferi, the perfect inverted repeat is 14 base pairs in length. In various mesophilic bacteriophages, the perfect inverted repeat is 22 base pairs or greater in length. Also, in some cases, e.g E. coli N15, the central perfect inverted palindrome is flanked by inverted repeat sequences, i.e forming part of a larger imperfect inverted palindrome.

The term “strand displacement” is used herein to describe the ability of a DNA polymerase to displace complementary strands on encountering a region of double stranded DNA during DNA synthesis.

The term “contacting” as used herein in connection with delivery of a nucleic acid (e.g., a template, DNA) to a host system (e.g, cell free or cellular) refers broadly to placing the nucleic acid (e.g., DNA template or plasmid template) into a host system such that it is present in the host system. Less broadly, contacting refers to any appropriate means of placing the template or plasmid template in a host system described herein. Contacting can be by such means that the template is appropriately transported into the interior of the cell such that, e.g., circular nucleic acid is produced by the host cell machinery. Such contacting may involve, for example transformation, transfection, electroporation, or lipofection.

### BRIEF DESCRIPTION OF THE DRAWINGS

FIG. 1 is an illustration of the generation of an embodiment of the covalently closed linear DNA vector construct of the invention through the activity of protelomerase. Protelomerase binds to the protelomerase binding sites, cleaves and ligates the two strands to generate covalently closed ends, leaving a partial protelomerase binding site at the ligated portion. In this embodiment, the partial protelomerase binding sites flank two DD-ITRs that flank a predetermined sequence (e.g. a transgene the encodes a protein or nucleic acid sequence). The vector is a covalently closed linear double stranded vector having hairpin ends in its natural state (annealed) and is capable of expressing transgene in a target cell. If the vector is fully denatured it would form a single stranded covalently closed circle having complimentary transgene sequences on one contiguous covalently closed single strand of DNA; upon renaturing the complimentary sequences would re-anneal. This is in contrast to a covalently closed circular double stranded DNA molecule that can express a transgene, e.g. plasmid DNA, where the complimentary sequences for expression of the transgene are on separate single stranded covalently closed molecules when denatured.

FIG. 2 is an illustration of the recombination of one embodiment of the DNA vector construct of the invention that can occur in vivo, resulting in concatemeric structures that persist within cells resulting in sustained gene expression.

FIG. 3 is an illustration of an embodiment of the DNA vector construct of the invention showing the DD-ITR resolution in vivo in the absence of an AAV Rep protein to generate structures that can either ligate in vivo after resolution or self-replicate in vivo, e.g. forming monomers and concatemeric structures, thereby resulting in sustained gene expression.

FIG. 4 is an illustration of the generation of another embodiment of the covalently closed DNA vector construct of the invention through the activity of protelomerase. Protelomerase binds to the protelomerase binding sites, cleaves and ligates the two strands to generate covalently closed ends, leaving a partial protelomerase binding site at the ligated portion. These constructs can also recombine in cells.

FIG. 5 lists chimeric ITRs for use in the instant invention. ITR2, ITR5, ITR5+2SNS, ITR2+5SNS, ITR5+2NS, ITR2+5NS, ITR2-TA, ITR5+TA, ITR2-GC, ITR5+GC, ITR2-2nt, ITR2 5nt, ITR2+7, ITR2 9nt, ITR2 10nt, ITR2 11nt, ITR2 15nt, ITR5 3nt, ITR5 Ent, ITR5 9 bp NS, ITR5 21nt, ITR5 30nt, ITR5 GAGY, ITR5 no GAGY, ITR2+8nt GAGY, ITR5 Spacer RBE, ITR2+8-8 Spacer RBE, ITR5 with ITR2 hairpins, ITR2 no hairpins, ITR2 T1, ITR2 T2, ITR2 T2 #2, ITR2 T3, ITR2 T4, ITR5+3nt Spacer & ITR5 NS, and ITR2 pHpa8. These ITRs were originally disclosed in U.S. Pat. No. 9,169,494, the contents of which are incorporated herein by reference in their entirety.

FIG. 6 is an illustration of the cloning of PCR-amplified and EcoRI cut double-D into EcoRI site of plasmid pGEM-3′Z.

FIG. 7 is a schematic of preparation of viral particles.

FIG. 8 shows protelomerase target site telRL. The DNA of phage N15 after infection consists of a circular DNA, which contains the telomere resolution site telRL. This site is a palindrome with telR and telL as right and left arms. Arrows indicate the inverted repeats (28 bp each) and dots interruptions in the palindrome. The central 22-bp palindrome telO is required for recognition and cleavage by the protelomerase, TelN. It cleaves the DNA within telO and joins the resulting ends to form two covalently closed hairpins at either end. The 28-bp sequences telR and telL designate also the ends of the linear N15 prophage DNA (“doggybone”).

FIGS. 9A-9C show results expected from expression of EGFP from mini lc-DNA. (FIG. 9A) In plasmid pDD3 (left), two telRL sites flank the transcriptional unit for EGFP. The reaction of pDD3 with protelomerase results in two linear closed linear DNA fragments, the EGFP-expressing mini lc-DNA (right), and the vector backbone (not shown). Two MunI recognition sites within the protelomerase target sites are used to generate the mini lo-DNA of the same size as a control. (FIG. 9B) Aliquots (0.2 μg each) of the preparations of the mini lc-DNA (lc) and the mini lo-DNA (10) will be subjected to 1.2% agarose gel electrophoresis. The parental plasmid (ccc) will be cut with either TelN (lc) or MunI (10) into two fragments of identical length (fraction I). Then the vector backbone digested using HaeII (fraction II), and the mini DNA purified (fraction III). The absence of vector DNA will be verified by treatment with HaeII to test for vector-derived digestion products. PstI will be used to test for remaining vector fragments after digestion of the mini DNAs (fraction III). To demonstrate that PstI cuts the parental ccc-DNA only once, the parental plasmid (ccc) will be included. (FIG. 9C) HEK293 cells will be transiently transfected with DNA (0.7 μg total) containing mini lc-DNA (lc), mini lo-DNA (lo), or parental DNA (ccc) (6 nmol each), respectively. As a control vector (c) will be used. Transfection efficiency will b controlled by cotransfection with EGFP-CNK.CT-expressing plasmid (pEGFPc2-CNK.CT, 0.35 μg). The total amount of DNA will be adjusted with vector DNA. The presence of EGFP and EGFP-CNK.CT will be determined 48 h post transfection by Western blot analysis. The expected bands representing EGFP and EGFP-CNK.CT are indicated.

FIG. 10 shows the expected results when conditional processing of the parent plasmid DNA vectors containing at least one DD-ITR further comprising a promoter operably linked to a predetermined DNA sequence occurs (FIG. 10A). R-cell conditional processing of the parent plasmid into mini DNA vectors. Under induced conditions, R-cells lead to the production of mini lcc (Tel-cell) and mini ccc (Cre-cell) DNA. (FIG. 10B). Processing of the parent plasmid construct into mini lcc and mini cc vectors. Efficiency of processing of the plasmid into lcc mini vectors (Tel) and ccc mini vectors (Cre) after plasmid extraction from R-cells under induced (42° C.) conditions. Schematics adjacent to each representative band show the expected plasmid and expected conformation they represent (FIG. 10C). In vivo Tel/pal recombination efficiency versus TelN/telRL. Efficiency of processing of the pDD4 plasmid into lcc mini vectors in Tel⁺ versus TelN⁺ R-cells.

FIG. 11 presents an exemplary schematic for manufacturing a circular nucleic acid having, in the 5′ to 3′ direction, a BAMHI restriction site, an F1 ORI, an DD-ITR (mutant), a promoter linked to a transgene (indicated by red star), a DD-ITR (mutant), a promotor linked to a transgene (indicated by red star), a DD-ITR (mutant), and a HINDIII restriction site, and having adaptor sequences ligated at each end via the restriction sites. A plasmid fragment is excised from the plasmid via cutting with a BAMHI and HINDIII restriction enzyme. Adaptor sequences are ligated to the plasmid fragment, forming the template. The template can be replicated in vitro or in vivo, e.g., in E. coli cells, cell extracts (e.g., E. coli cell extract), or bacterial packaging cells. The exemplified order of the elements (e.g., a cleavage site, an ORI, an ITR, or a promoter linked to a transgene) shown herein from 5′ to 3′ is not meant to be limiting, and it is to be understood the elements can be in any order from 5′ to 3′.

FIG. 12 presents an exemplary schematic for manufacturing a circular nucleic acid having, in the 5′ to 3′ direction, a BAMHI restriction site, an F1 ORI, an DD-ITR (mutant), a promotor linked to a transgene (indicated by star), a DD-ITR (mutant), and a HINDIII restriction site, and having adaptor sequences ligated at each end via the restriction sites. A plasmid fragment is excised from the plasmid via cutting with a BAMHI and HINDIII restriction enzyme. Adaptor sequences are ligated to the plasmid fragment, forming the template. The template can be replicated in vitro or in vivo, e.g., in E. coli cells, cell extracts (e.g., E. coli cell extract), or bacterial packaging cells. The exemplified order of the elements (e.g., a cleavage site, an ORI, an ITR, or a promoter linked to a transgene) shown herein from 5′ to 3′ is not meant to be limiting, and it is to be understood the elements can be in any order from 5′ to 3′.

FIG. 13 presents an exemplary schematic for manufacturing a circular nucleic acid having, in the 5′ to 3′ direction, a BAMHI restriction site, an F1 ORI, a PVUII restriction site, an DD-ITR (mutant), a promotor linked to a transgene (indicated by star), DD-ITR (mutant), and a HINDIII restriction site, and having adaptor sequences ligated at each end via the restriction sites. A plasmid fragment is excised from the plasmid via cutting with a BAMHI and HINDIII restriction enzyme. Adaptor sequences are ligated to the plasmid fragment, forming the template. The template can be replicated in vitro or in vivo, e.g., in E. coli cells, cell extracts (e.g., E. coli cell extract), or bacterial packaging cells. Following replication, the circular nucleic acid is further cut with a PVUII restriction enzyme, removing an adaptor sequence and the ORI, and resulting in one open end, and one closed end. The exemplified order of the elements (e.g., a cleavage site, an ORI, an ITR, or a promoter linked to a transgene) shown herein from 5′ to 3′ is not meant to be limiting, and it is to be understood the elements can be in any order from 5′ to 3′.

FIG. 14 presents an exemplary schematic of manufacturing a self-complementary, single stranded DNA vector having in the 5′ to 3′ direction, an F1 ORI, an DD-ITR (mutant), a promotor linked to a transgene (indicated by star), a DD-ITR (mutant), a promotor linked to a transgene (indicated by star), a DD-ITR (mutant), a hairpin sequence, a complementary DD-ITR (mutant), a complementary promotor linked to a transgene (indicated by star), a complementary DD-ITR (mutant), a complementary promotor linked to a transgene (indicated by star), a complementary DD-ITR (mutant), and a ORIΔ29. This method uses a bacterial packaging cell and a helper phage. Asterisk indicates a complementary sequence, e.g., a complementary TR or transgene sequence. The exemplified order of the elements (e.g., a cleavage site, an ORI, an ITR, or a promoter linked to a transgene) shown herein from 5′ to 3′ is not meant to be limiting, and it is to be understood the elements can be in any order from 5′ to 3′.

### DETAILED DESCRIPTION OF THE INVENTION

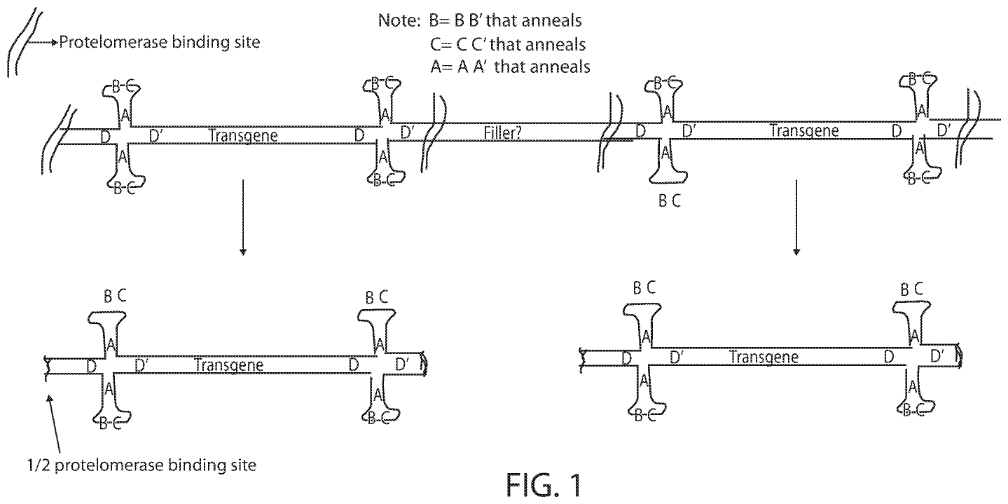

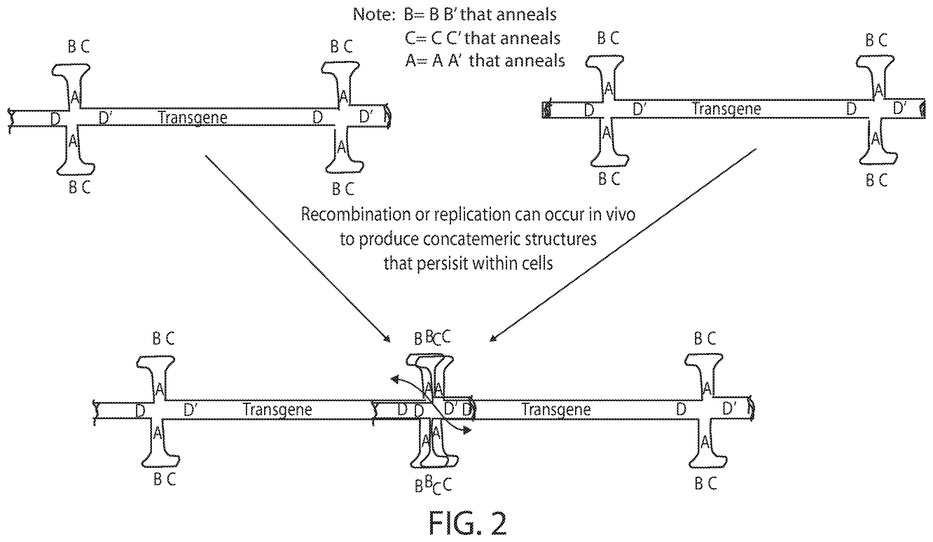

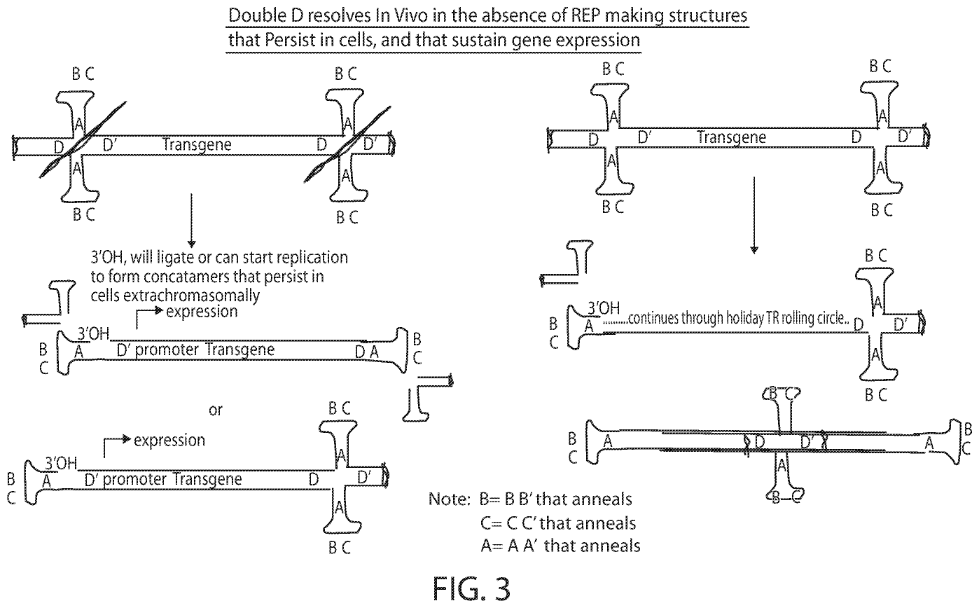

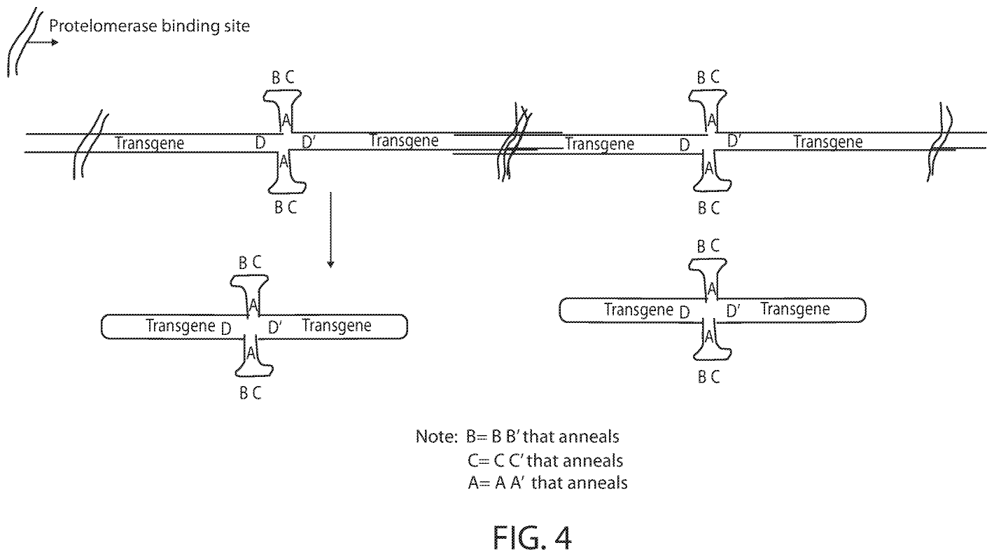

Aspects of the invention relate to a DNA vector for delivery of a predetermined nucleic acid sequence into a target cell for sustained expression. The DNA vector comprises at least one DD-ITRs and a predetermined nucleic acid sequence/construct (e.g., a regulatory sequence such as a promoter operably linked to a predetermined DNA sequence (e.g. a heterologous nucleic acid) that can anneal into expressible ds DNA). The DNA vector is a covalently closed linear non-viral DNA construct. The DNA vector can further self-replicate and/or recombine in the target cell (e.g., when the target cell divides). The DNA vector construct having at least one DD-ITR, for delivery of nucleic acid to cells is linear DNA that has covalently closed hairpin ends and is not plasmid DNA, i.e. is not a DD-ITR plasmid. In one preferred embodiment, the covalently closed linear DNA vector comprises two DD-ITRs (e.g. as shown in FIG. 1).

An ITR (inverted terminal repeat) typically has regions of complementary palindromic sequences which can anneal, with intervening spacer sequences that allow the formation of a T or Y structure. Such regions are known in the art and referred herein as A, A′, B, B′, C, C′ and a D region (E.g. as described in U.S. Pat. No. 5,478,745). In the context of an AAV ITR, the D region that is a single stranded part of the genome. A DD-ITR has an additional D′ region which is complementary to the D region of the ITR (See also, Xiao et al. J. Virology vo. 71 (2). 1997: p941-948, incorporated herein by reference in its' entirety).

In some embodiments, there are two DD-ITR's, located such that they flank the predetermined nucleic acid sequence/construct (e.g., when viewed as shown in the bottom of FIG. 1). In some embodiment, at least one of the DD-ITR's is an AAV ITR.

In some embodiments, the DNA construct further comprises a partial protelomerase binding site (e.g., such as is left following generation of covalently closed ends by a protelomerase enzyme activity in vitro).

In one preferred embodiment, the covalently closed non-viral DNA construct comprises two DD-ITRs, e.g. as shown in the bottom of FIG. 1. While FIG. 1 shows half of a protelomerase binding site, the covalently closed non-viral DNA construct that comprises two DD-ITRs need not comprise a protelomerase binding site.

In embodiments of the invention there are at least 2 nucleotides located between the 5′ end of the promoter (+ strand) and the DD-ITRs to serve as spacers. These spacers prevent the DD-ITR from inhibiting promoter function. The spacer can be larger than 2 nucleotides, e.g., 5 or more nucleotides. In one embodiment, the spacer is at least 10 nucleotides, in one embodiment the spacer is 20 nucleotides or more, or 25 nucleotides or more. In one embodiment the spacer is from 5-50 nucleotides. In a preferred embodiment, the spacer is 20 nucleotides.

In embodiments of the invention, there is no promoter in the vector, and the DD-ITR acts as a promoter, to drive expression of the predetermined DNA nucleic acid. In some embodiments, there are at least 2 nucleotides located between the 5′ end of the predetermined nucleic acid (+ strand) and the DD-ITRs (the D region) to serve as spacers. The spacer can be larger than 2 nucleotides, e.g., 5 or more nucleotides. In one embodiment, the spacer is at least 10 nucleotides, in one embodiment the spacer is 20 nucleotides or more, or 25 nucleotides or more. In one embodiment the spacer is from 5-50 nucleotides.

In embodiments of the invention, the DNA construct/vector lacks sequences that encode an AAV Rep protein (e.g., a Rep protein corresponding to the AAV from which the DD-ITR is derived).

In some embodiments, the majority of the sequence of the DNA construct/vector is mammalian DNA instead of bacterial DNA. The construct is closed linear double stranded DNA that expresses a heterologous nucleic acid and is not closed circular double stranded plasmid DNA that can express a heterologous gene.

The ITR can be from any parvovirus, for example a dependovirus such as AAV. Numerous serotypes of AAV are known. See for example Table 1 below

TABLE 1

| Complete Genomes | GenBank ® Database Accession Number |
|---|---|
| Adeno-associated virus 1 | NC_002077, AF063497 |
| Adeno-associated virus 2 | NC_001401 |
| Adeno-associated virus 3 | NC_001729 |
| Adeno-associated virus 3B | NC_001863 |
| Adeno-associated virus 4 | NC_001829 |
| Adeno-associated virus 5 | Y18065, AF085716 |
| Adeno-associated virus 6 | NC_001862 |
| Adeno-associated virus 8 | NC_006261 |
| Adeno-associated virus 9 | AX753250.1 |
| Avian AAV ATCC VR-865 | AY186198, AY629583, NC_004828 |
| Avian AAV strain DA-1 | NC_006263, AY629583 |
| Bovine AAV | NC_005889, AY388617 |

Clade A

| | |
|---|---|
| AAV1 | NC_002077, AF063497 |
| AAV6 | NC_001862 |
| Hu.48 | AY530611 |
| Hu 43 | AY530606 |
| Hu 44 | AY530607 |
| Hu 46 | AY530609 |

Clade B

| | |
|---|---|
| Hu. 19 | AY530584 |
| Hu. 20 | AY530586 |
| Hu 23 | AY530589 |
| Hu22 | AY530588 |
| Hu24 | AY530590 |
| Hu21 | AY530587 |
| Hu27 | AY530592 |
| Hu28 | AY530593 |
| Hu29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |

Clade C

| | |
|---|---|
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |

Clade D

| | |
|---|---|
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |

Clade E

| | |
|---|---|
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |

Clade F

| | |
|---|---|
| Hu14 (AAV9) | AY530579 |
| Hu31 | AY530596 |
| Hu32 | AY530597 |

Clonal Isolate

| | |
|---|---|
| AAV5 | Y18065, AF085716 |
| AAV 3 | NC_001729 |
| AAV 3B | NC_001863 |
| AAV4 | NC_001829 |
| Rh34 | AY243001 |
| Rh33 | AY243002 |
| Rh32 | AY243003 |

In embodiments, the DNA construct/vector persist (e.g., for an extended period of time) in the target cell (e.g., via replication or recombination, and/or concatemer formation). This may occur through self-replication when the target cell divides and/or concatemer formation, or a combination of both. In embodiments of the invention, the DNA construct/vector is converted into a concatemeric structures within a target cell. In embodiments, the concatemeric structures persist (e.g., for an extended period of time) in the target cell (e.g., via replication or recombination). Persistence of the concatemeric structure may be extra-chromosomal (e.g., as a mini-chromosome) or by integration into the target cell chromosome.

Another aspect of the invention relates to a method for introducing a nucleic acid construct into a target cell for sustained expression. The method comprises administering to the target cell a covalently closed non-viral DNA construct described herein. Administration to the target cell can be in vitro, in vivo, or ex vivo. Successful introduction of the DNA construct into the target cell promotes expression of the nucleic acid construct. In certain embodiments, the ability of the DNA construct to replicate or integrate into the target cell genome leads to the expression being sustained.

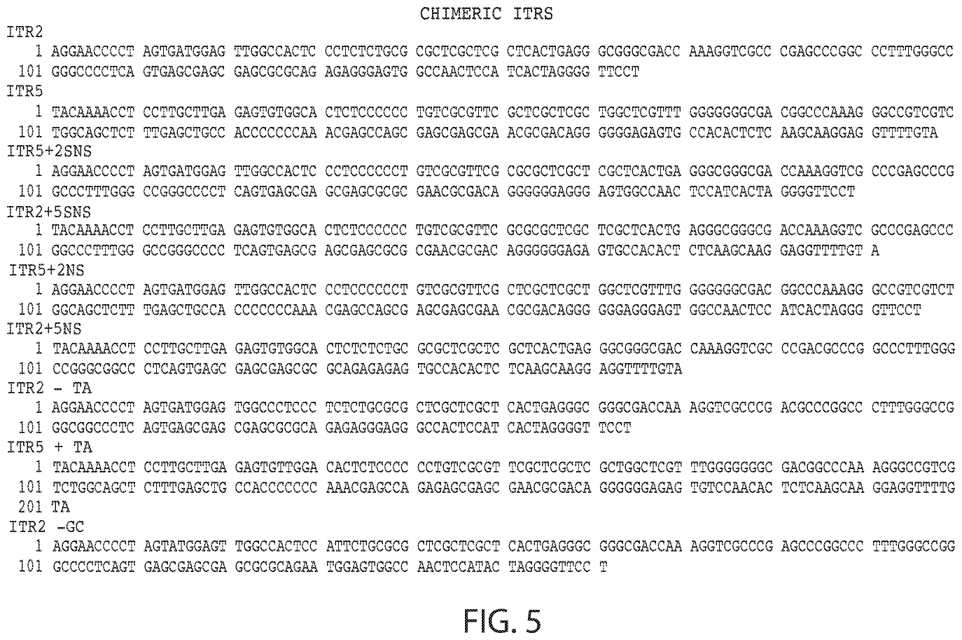

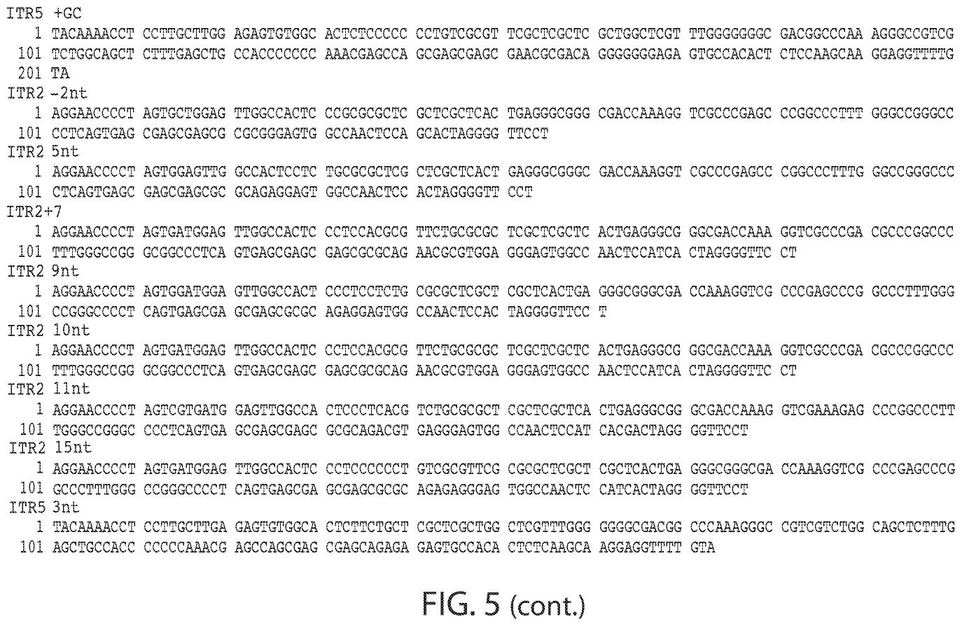

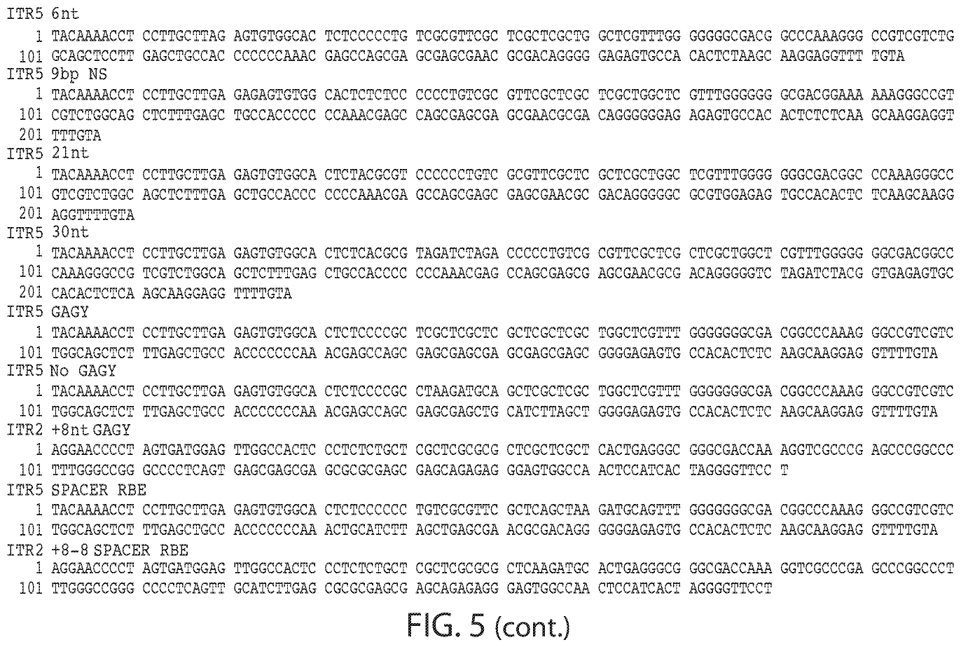

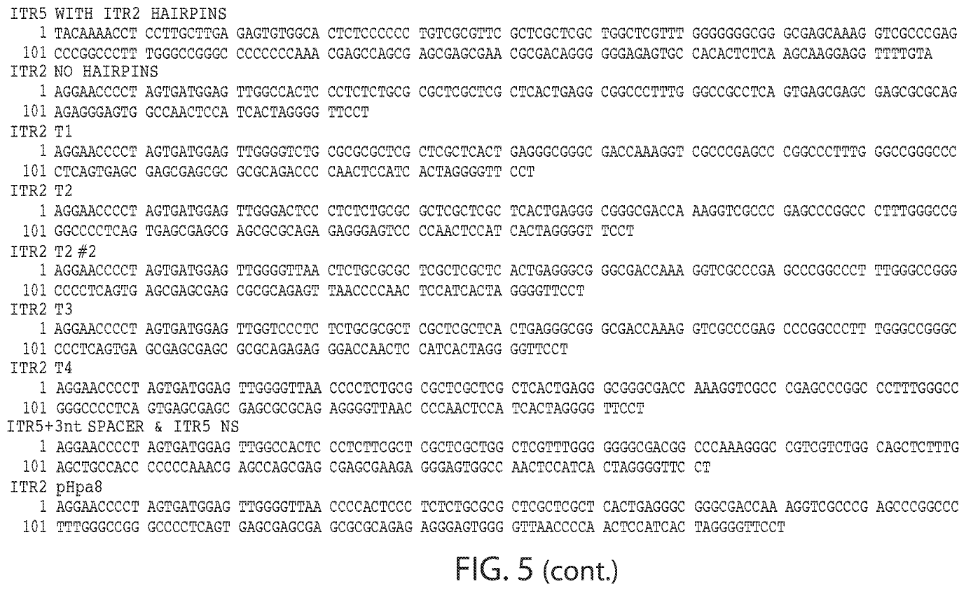

Another aspect of the invention relates to a method for introducing a nucleic acid into a target cell for sustained expression by administering a covalently closed non-viral DNA construct comprising at least one ITR sequence and complementary strands of the nucleic acid construct that anneal into a linear dsDNA that is expressible. The DNA construct is similar to those described above, in that it forms linear DNA with hairpin covalently closed ends. It may further comprise a partial protelomerase recognition sequence (reflective of it being generated through the protelomerase activity). The ITR is a synthetic ITR such as those shown in FIG. 5. The ITR sequence may further comprise both D and D′ sequences located on either side of the hairpin structure, as described herein.

Another aspect of the invention relates to a cell or population thereof, generated from introduction of a DNA construct described herein. A significant portion of the population receives the DNA construct and expresses the encoded nucleic acid. In embodiments, at least 10% of the cells of the population express the introduced nucleic acid. In embodiments, at least 20%, 30%, 40%, 50% or more express the introduced nucleic acid. In embodiments, at least 60%, 70%, 80% or more of the cells in the population express the introduced nucleic acid.

The methods and DNA constructs/vectors described herein promote sustained expression of a predetermined nucleic acid in a target cell or population thereof. By sustained expression is meant that the expression of encoded product, e.g., protein or nucleic acid, is at a detectable level that persists for an extended period of time, if not indefinitely, following administration of the subject vector. By extended period of time is meant from 1-5 weeks, from 2-5 weeks, from 3-5 weeks, from 4-5 weeks, at least 5 weeks, at least 6 weeks, at least 1 month, at least 2 months, at least 3 months, at least 4 months, at least 5 months, at least 6 months, at least 7, 8, 9, 10, 11 or 12 months, from 1-12 months, from 1-10 years, at least 1, 2, 3, 4, 5, 6, 7, 8, 9, or 10 years, or longer. By detectable level is meant that the expression of the encoded product is at a level such that one can detect the encoded product in target cell, or the mammal comprising the same, e.g., in the serum of the mammal, at a therapeutic concentration. As compared to a control in which the pBluescript plasmid vector (Stratagene Corporation, La Jolla, Calif.) is employed, protein expression persists for a period of time at a detectable level that is at least about 2 fold, usually at least about 5 fold and more usually at least about 10 fold longer following the subject methods as compared to a control. An encoded product is considered to be at a detectable level if it can be detected using technology and protocols readily available and well known to those of skill in the art.

In some embodiments, the above-described sustained expression is not only at a detectable level, but at a high level. A minimal vector is considered to provide for a high level of expression if, after a period of time following its administration, e.g., at least about 28 days, the protein or nucleic acid encoded by the expression cassette of the vector is present at high levels in the host, e.g., in the target cells, in the serum of the host, etc. Levels of an encoded product are considered “high” for purposes of the invention if they are present in amounts such that they exhibit detectable activity (e.g., have an impact on the phenotype), e.g., therapeutic activity, in the host. Whether or not the expression level of a particular product is high will necessarily vary depending on the nature of the particular product, but can readily be determined by those of skill in the art, e.g., by an evaluation of whether expression of the product is sufficient to exhibit a desired effect on the phenotype of the host, such as an amelioration of a disease symptom.

Generation of Covalently Closed Ends

Covalently closed ends of the DNA construct described herein may be generated by a variety of known methods, including in vitro cell-free synthesis. One method of generating the covalently closed ends is by incorporation of protelomerase binding sites in a precursor molecule, and exposure of the molecule to protelomerase to thereby cleave and ligate the DNA at the site. Non-limiting examples of cell free in vitro synthesis are e.g. described in U.S. Pat. Nos. 9,109,250; 6,451,563; Nucleic Acids Res. 2015 Oct. 15; 43(18): e120; U.S. Pat. Nos. 9,499,847; 15/508,766; PCT/GB2017/052413; and Antisense & nucleic acid drug development 11:149-153 (2001); herein incorporated by reference in their entirety.

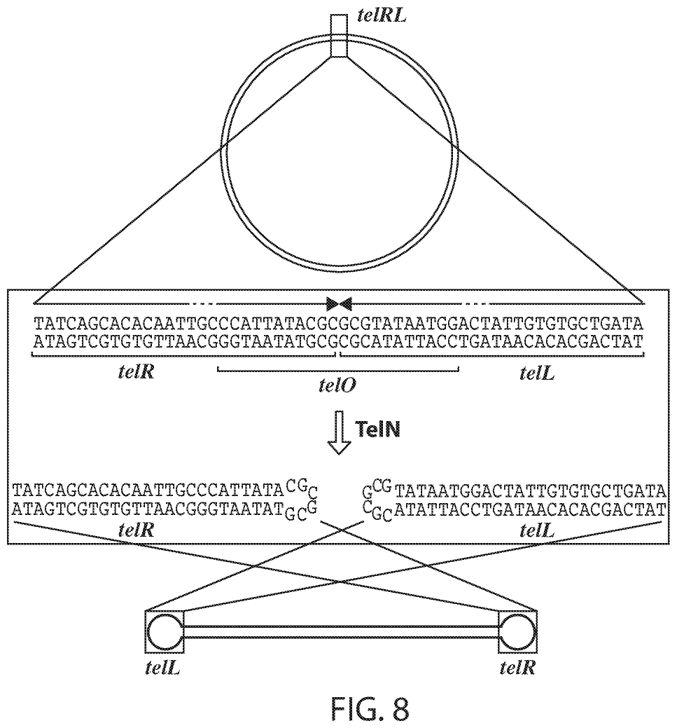

One can design a recombinant AAV DNA template having wild type ITRs, synthetic ITRs, or DD ITR sequences, or a combination thereof, together within imperfect palindromic structure containing protelomerase sites such as telRL (see FIG. 8) The template is used to produce closed linear vector.

In one embodiment, the viral vector template comprises wild type ITRs, e.g., that can have deletions, insertions, or substitutions. In one embodiment, the vector template comprises at least one synthetic ITR (e.g. non-limiting examples are those described in FIG. 5), an expression cassette, and flanked on each side of the ITRs a telomerase target site, which can be cleaved by a telomerase that covalently closes the ends. In one embodiment, the vector comprises two DD ITRs, an expression cassette, and flanked on each side of the DD ITRs is a telomerase target site, which can be cleaved by the telomerase and covalently closes the ends.

A prokaryotic system can be used herein. In lysogenic bacteria, the bacteriophage N15 exists as a linear extrachromosomal DNA with covalently closed ends (see Rybchin V N, Svarchevsky A N (1999) The plasmid prophage N15: a linear DNA with covalently closed ends. Mol Micro-biol 33:895-903). This DNA arises by a cleaving joining reaction, which is exerted by a single enzyme, a protelomerase, for example, TelN (prokaryotic telomerase) [Deneke J, Ziegelin G, Lurz R, Lanka E (2000) The protelomerase of temperate Escherichia coli phage N15 has cleaving joining activity. Proc Natl Acad Sci USA 97:7721-7726]. A protelomerase such as TelN recognizes a target sequence in double-stranded DNA. The target site is an imperfect palindromic structure termed telRL (see FIG. 8), which is formed by the two halves telR and telL, corresponding to the covalently closed ends of the linear prophage. The enzyme cleaves both DNA strands and joins the resulting ends to form covalently closed hairpin structures. The resulting DNA molecule has two hairpin loops (FIG. 8). TelN is able to linearize a recombinant plasmid harboring the telRL site [Deneke J, Ziegelin G, Lurz R, Lanka E (2000) The protelom-erase of temperate Escherichia coli phage N15 has cleaving-joining activity. Proc Natl Acad Sci USA 97:7721-7726]. Therefore, one can employee this enzyme on a plasmid DNA for expression in higher organisms.

In one embodiment, a method is provided herein that efficiently produces rAAV closed linear DNA in an in vivo cell system that can be used to make rAAV virus. The method comprises using a cell that expresses a protelomerase, such as TelN, or other protelomerase, wherein the protelomerase gene is under the control of a regulatable promoter. For example, an inducible promoter such as a small molecule regulated promoter or a temperature sensitive promoter, e.g. a heat shock promoter. After sufficient production of the AAV template DNA, one can allow the protelomerase to be expressed which will excise the closed linear AAV genome from the template, e.g containing a double D, synthetic, or AAV ITR. In an alternative embodiment the protelomerase can be added to the cell by known means. The closed linear AAV genome containing the ITRs can then be used in place of plasmid DNA in a transfection protocol to make rAAV virus. The closed linear genome comprises a ½ protelomerase binding site on each end.

In certain embodiments, the in-vivo cell system is used to produce a non-viral DNA vector construct for delivery of a predetermined nucleic acid sequence into a target cell for sustained expression. The non-viral DNA vector comprises, two DD-ITRs each comprising: an inverted terminal repeat having an A, A′, B, B′, C, C′ and D region; a D′ region; and wherein the D and D′ region are complementary palindromic sequences of about 5-20 nt in length, are positioned adjacent the A and A′ region; the predetermined nucleic acid sequence (e.g. a heterologous gene for expression); wherein the two DD-ITRs flank the nucleic acid in the context of covalently closed non-viral DNA and wherein the closed linear vector comprises a ½ protelomerase binding site on each end.

The TelN/telRL system described herein can be used to produce the closed linear DNA fragments either by linearizing a parental plasmid containing one telRL site or by excising the rAAV DNA fragment, or non-viral vector fragment, comprising a promoter, the gene of interest, a polyadenylation signal from the parental plasmid with two flanking ITRs, further having two telRL sites flanking the respective segment. In one embodiment, there is at least one double “D” ITR. The resulting linear covalently closed DNA molecules are functional in vivo.

The system comprises recombinant host cells. Suitable host cells for use in the present production system include microbial cells, for example, bacterial cells such as E. coli cells, and yeast cells such as S. cerevisiae. Mammalian host cells may also be used including Chinese hamster ovary (CHO) cell for example of K1 lineage (ATCC CCL 61) including the Pro5 variant (ATCC CRL 1281); the fibroblast-like cells derived from SV40-transformed African Green monkey kidney of the CV-1 lineage (ATCC CCL 70), of the COS-1 lineage (ATCC CRL 1650) and of the COS-7 lineage (ATCC CRL 1651; murine L-cells, murine 3T3 cells (ATCC CRL 1658), murine C127 cells, human embryonic kidney cells of the 293 lineage (ATCC CRL 1573), human carcinoma cells including those of the HeLa lineage (ATCC CCL 2), and neuroblastoma cells of the lines IMR-32 (ATCC CCL 127), SK-N-MC (ATCC HTB 10) and SK-N-SH (ATCC HTB 11).

The host cell is designed to encode at least one recombinase. The host cell may also be designed to encode two or multiple recombinases. The term “recombinase” refers to an enzyme that catalyzes DNA exchange at a specific target site, for example, a palindromic sequence, by excision/insertion, inversion, translocation and exchange. Examples of suitable recombinases for use in the present system include, but are not limited to, TelN, Tel, Tel (gp26 K02 phage) Cre, Flp, phiC31, Int and other lambdoid phage integrases, e.g. phi 80, HK022 and HP1 recombinases. The target sequences for each of these recombinases are, respectively:

the telRL site
(SEQ ID NO: 31)
(TATCAGCACACAATTGCCCATTATACGCGCGTATAATGGACTAT
TGTGTGCTGA);
the pal site
(SEQ ID NO: 32)
(ACCTATTTCAGCATACTACGCGCGTAGTATGCTGAAATAGGT);
the φK02 telRL site
(SEQ ID NO: 2)
(CCATTATACGCGCGTATAATGG),
the loxP site
(SEQ ID NO: 33)
(TAACT TC GTATAGCATACATTATACGAAGTTAT);
the FRT site
(SEQ ID NO: 34)
(GAAGTTCCTATTCTCTAGAAAGTATAGGAACTTC);
the phiC31 attP site
(SEQ ID NO: 35)
(CCCAGGTCAGAAGCGGTTTTCGGGAGTAGTGCCCCAACTGGGGTAAC
CTTTGAGTTCTCTCAGTT
GGGGGCGTAGGGTCGCCGACAYGACACAAGGGGTT);
the λ attP site
(SEQ ID NO: 36)
(TGATAGTGACCTGTTCGTTGCAACACATTGATGAGCAATGCTTTTTTAT
AATGCCAACTTTGTACAAAA
AAGCTGAACGAGAAACGTAAAATGATATAAA).

Expression of the recombinase is under the control of any regulated or inducible promoter, i.e. a promoter which is activated under a particular physical or chemical condition or stimulus. Examples of suitable promoters include thermally-regulated promoters such as the λpL promoter, the IPTG regulated lac promoter, the glucose regulated ara promoter, the T7 polymerase regulated promoter, cold-shock inducible cspA promoter, pH inducible promoters, or combinations thereof, such as tac (T7 and lac) dual regulated promoter.

The recombinant cells also incorporate at least one DD-ITR and an expression cassette adapted to express a nucleic acid of interest. The DD-ITR containing expression vector includes regulatory expression sequences, e.g. promoter, initiation and termination sequences, and a nucleic acid sequence of interest appropriately positioned relative to the regulatory sequences so that expression of the nucleic acid of interest will occur, i.e. the nucleic acid of interest is expressibly incorporated within the expression vector as well as at least one flanking DD-ITR, preferably flanked on both ends by DD-ITRs. In one embodiment, one of the DD-ITRs can serve as the promoter. The regulatory expression sequences and nucleic acid sequence of interest, i.e. the expression cassette, is flanked on either side by a target sequence (e.g. target nucleic acid sequence) for at least a first recombinase.

Alternate methods of generating covalently closed ends are known in the art e.g., by formation of mini-circle DNA from plasmids (e.g. as described in U.S. Pat. Nos. 8,828,726, and 7,897,380, the contents of each of which are incorporated by reference in their entirety). For example, one method of cell-free synthesis of the DD-vectors combines the use of two enzymes—Phi29 DNA polymerase and a protelomerase, and generates high fidelity, covalently closed, linear DNA constructs. The constructs contain no antibiotic resistance markers, and therefore eliminate the packaging of these sequences. The process can amplify DD-AAV constructs in a 2-week process at commercial scale and maintain the ITR sequences required for virus production.

Phi29 DNA polymerase is used to amplify double-stranded DNA by rolling circle amplification, and a protelomerase to generate covalently closed linear DNA, which coupled with a streamlined purification process, results in a pure DNA product containing just the sequence of interest.

Phi29 DNA polymerase has high fidelity ( 1/10⁶- 1/10⁷) and high processivity (approximately 70 kbp). These features make this polymerase particularly suitable for the large-scale production of GMP DNA. Protelomerases (also known as telomere resolvases) catalyze the formation of covalently closed hairpin ends on linear DNA and have been identified in some phages, bacterial plasmids and bacterial chromosomes. A pair of protelomerases recognizes inverted palindromic DNA recognition sequences and catalyzes strand breakage, strand exchange and DNA ligation to generate closed linear hairpin ends. The formation of these closed ended structures makes the DNA resistant to exonuclease activity, allowing for simple purification and can improve stability and duration of expression.

In one embodiment of synthesis, the production of DD-constructs depends upon the introduction of protelomerase recognition sequences that flank the region of interest. These sites are palindromic sequences (e.g. 56 bp) that are highly specific to particular protelomerase enzymes. The protelomerase proteins bind to these sites to perform a cleavage joining reaction that results in monomeric double-stranded, linear, covalently closed DNA constructs. The DNA outside the gene of interest (e.g., the original vector backbone) will also be similarly processed by this enzyme, these regions can be removed by the sequential action of restriction enzymes cutting at restriction sites unique to the vector backbone and exonuclease digestion of the released fragments, leaving only the desired covalently closed linear DNA containing the DD regions.

When denatured, the DD-constructs comprise circular DNA molecules that can be used as a starting material for further amplification reactions.

Protelomerase Binding Sites

In one embodiment, the DNA construct comprises a protelomerase binding site and the covalently closed ends are formed by protelomorase enzyme activity (e.g., in vitro). Protelomerase binding sites and corresponding protelomerases for use in the invention are provided in U.S. Pat. Nos. 9,499,847 and 9,190,250, the contents of each of which are incorporated herein by reference in their entirety. A protelomerase target sequence as used in the invention preferably comprises a double stranded palindromic (perfect inverted repeat) sequence of at least 14 base pairs in length. Preferred perfect inverted repeat sequences include the sequences of SEQ ID NOs: 1 to 6 and variants thereof. SEQ ID NO: 1 (NCATNNTANNCGNNTANNATGN) is a 22 base consensus sequence for a mesophilic bacteriophage perfect inverted repeat. Base pairs of the perfect inverted repeat are conserved at certain positions between different bacteriophages, while flexibility in sequence is possible at other positions. Thus, SEQ ID NO: 1 is a minimum consensus sequence for a perfect inverted repeat sequence for use with a bacteriophage protelomerase in the process of the present invention.

Within the consensus defined by SEQ ID NO: 1, SEQ ID NO: 2 (CCATTATACGCGCGTATAATGG) is a particularly preferred perfect inverted repeat sequence for use with E. coli phage N15, and Klebsiella phage Phi KO2 protelomerases. Also within the consensus defined by SEQ ID NO: 1, SEQ ID NOs: 3 to 5: SEQ ID NO: 3 (GCATACTACGCGCGTAGTATGC), SEQ ID NO: 4 (CCATACTATACGTATAGTATGG), SEQ ID NO: 5 (GCATACTATACGTATAGTATGC), are particularly preferred perfect inverted repeat sequences for use respectively with protelomerases from Yersinia phage PY54, Halomonas phage phiHAP-1, and Vibrio phage VP882. SEQ ID NO: 6 (ATTATATATATAAT) is a particularly preferred perfect inverted repeat sequence for use with a Borrelia burgdorferi protelomerase. This perfect inverted repeat sequence is from a linear covalently closed plasmid, lpB31.16 comprised in Borrelia burgdorferi. This 14 base sequence is shorter than the 22 bp consensus perfect inverted repeat for bacteriophages (SEQ ID NO: 1), indicating that bacterial protelomerases may differ in specific target sequence requirements to bacteriophage protelomerases. However, all protelomerase target sequences share the common structural motif of a perfect inverted repeat.

The perfect inverted repeat sequence may be greater than 22 bp in length depending on the requirements of the specific protelomerase used in the process of the invention. Thus, in some embodiments, the perfect inverted repeat may be at least 30, at least 40, at least 60, at least 80 or at least 100 base pairs in length. Examples of such perfect inverted repeat sequences include SEQ ID NOs: 7 to 9 and variants thereof. SEQ ID NO: 7 (GGCATAC TATACGTATAGTATGCC); SEQ ID NO: 8 (ACCTATTTCAGCATACTACGCGCG-TAGTATGCTGAAATAGGT); SEQ ID NO: 9 (CCTATATTGGGCCACCTATGTATG-CACAGTTCGCCCATACTATACGTATAGTATGGGCGAACTGTGCATACATAGGTGGCC CAATATAGG). SEQ ID NOs: 7 to 9 and variants thereof are particularly preferred for use respectively with protelomerases from Vibrio phage VP882, Yersinia phage PY54 and Halomonas phage phi HAP-1.

The perfect inverted repeat may be flanked by additional inverted repeat sequences. The flanking inverted repeats may be perfect or imperfect repeats i.e may be completely symmetrical or partially symmetrical. The flanking inverted repeats may be contiguous with or non-contiguous with the central palindrome. The protelomerase target sequence may comprise an imperfect inverted repeat sequence which comprises a perfect inverted repeat sequence of at least 14 base pairs in length. An example is SEQ ID NO: 14. The imperfect inverted repeat sequence may comprise a perfect inverted repeat sequence of at least 22 base pairs in length. An example is SEQ ID NO: 10.

Particularly preferred protelomerase target sequences comprise the sequences of SEQ ID NOs: 10 to 14 or variants thereof. SEQ ID NO: 10:

SEQ ID NO: 11
(TATCAGCACACAATTGCCCATTATACG-CGCGTATAATGGACTATTG
TGTGCTGATA);
SEQ ID NO: 12
(ATGCGCGCATCCATTATACGCGCGTATAATGGCGATAATACA);
SEQ ID NO: 13:
(TAGTCACCTATTTCAGCATACTACGCGCGTAGTATGCTGAAATAGGT
TACTG);
(GGGATCCCGTTCCATACATACATGTATCCATGTGGCATACTATACG
TATAGTATGCCGATGTTACATATGGTATCATTCGGGATCCCGTT);
SEQ ID NO: 14
(TACTAAATAAATATTATATATATAATTTTTTATTAGTA).

The sequences of SEQ ID NOs: 10 to 14 comprise perfect inverted repeat sequences as described above, and additionally comprise flanking sequences from the relevant organisms. A protelomerase target sequence comprising the sequence of SEQ ID NO: 10 or a variant thereof is preferred for use in combination with E. coli N15 TelN protelomerase and variants thereof. A protelomerase target sequence comprising the sequence of SEQ ID NO: 11 or a variant thereof is preferred for use in combination with Klebsiella phage Phi K02 protelomerase and variants thereof. A protelomerase target sequence comprising the sequence of SEQ ID NO: 12 or a variant thereof is preferred for use in combination with Yersinia phage PY54 protelomerase and variants thereof. A protelomerase target sequence comprising the sequence of SEQ ID NO: 13 or a variant thereof is preferred for use in combination with Vibrio phage VP882 protelomerase and variants thereof. A protelomerase target sequence comprising the sequence of SEQ ID NO: 14 or a variant thereof is preferred for use in combination with a Borrelia burgdorferi protelomerase.

Variants of any of the palindrome or protelomerase target sequences described above include homologues or mutants thereof. Mutants include truncations, substitutions or deletions with respect to the native sequence. A variant sequence is any sequence whose presence in the DNA template allows for its conversion into a closed linear DNA by the enzymatic activity of protelomerase. This can readily be determined by use of an appropriate assay for the formation of closed linear DNA. Any suitable assay described in the art may be used. An example of a suitable assay is described in Deneke et al, PNAS (2000) 97, 7721-7726. Preferably, the variant allows for protelomerase binding and activity that is comparable to that observed with the native sequence. Examples of preferred variants of palindrome sequences described herein include truncated palindrome sequences that preserve the perfect repeat structure, and remain capable of allowing for formation of closed linear DNA. However, variant protelomerase target sequences may be modified such that they no longer preserve a perfect palindrome, provided that they are able to act as substrates for protelomerase activity.

It should be understood that the skilled person would readily be able to identify suitable protelomerase target sequences for use in the invention on the basis of the structural principles outlined above. Candidate protelomerase target sequences can be screened for their ability to promote formation of closed linear DNA using the assays described above.

Generation of Covalently Closed Linear DNA Construct

The covalently closed vectors described herein may be generated in vitro or in vivo. The vectors are covalently closed linear double stranded vectors capable of expressing transgene in a target cell. One example of an in vitro process for the production of a closed linear expression cassette DNA, e.g. containing the ITRs described herein, comprises a) contacting a DNA template comprising at least one expression cassette flanked on either side by a protelomerase target sequence with at least one DNA polymerase in the presence of one or more primers under conditions promoting amplification of said template; and b) contacting amplified DNA produced in a) with at least one, protelomerase under conditions promoting formation of a closed linear expression cassette DNA. The closed linear expression cassette DNA product may comprise, consist or consist essentially of a eukaryotic promoter operably linked to a coding sequence of interest, and optionally a eukaryotic transcription termination sequence. The closed linear expression cassette DNA product may additionally lack one or more bacterial or vector sequences, typically selected from the group consisting of: (i) bacterial origins of replication; (ii) bacterial selection markers (typically antibiotic resistance genes) and (iii) unmethylated CpG motifs.

As outlined above, any DNA template comprising at least one protelomerase target sequence may be amplified according to the process of the invention. Thus, although production of therapeutic DNA molecules, e.g. for DNA vaccines or other therapeutic proteins and nucleic acid is preferred, the process of the invention may be used to produce any type of closed linear DNA. The DNA template may be a double stranded (ds) or a single stranded (ss) DNA. A double stranded DNA template may be an open circular double stranded DNA, a closed circular double stranded DNA, an open linear double stranded DNA or a closed linear double stranded DNA. Preferably, the template is a closed circular double stranded DNA. Closed circular dsDNA templates are particularly preferred for use with RCA (rolling circle amplification) DNA polymerases. A circular dsDNA template may be in the form of a plasmid or other vector typically used to house a gene for bacterial propagation. Thus, the process of the invention may be used to amplify any commercially available plasmid or other vector, such as a commercially available DNA medicine, and then convert the amplified vector DNA into closed linear DNA.

An open circular dsDNA may be used as a template where the DNA polymerase is a strand displacement polymerase which can initiate amplification from at a nicked DNA strand. In this embodiment, the template may be previously incubated with one or more enzymes which nick a DNA strand in the template at one or more sites. A closed linear dsDNA may also be used as a template. The closed linear dsDNA template (starting material) may be identical to the closed linear DNA product. Where a closed linear DNA is used as a template, it may be incubated under denaturing conditions to form a single stranded circular DNA before or during conditions promoting amplification of the template DNA.

As outlined above, the DNA template typically comprises an expression cassette as described above, i.e comprising, consisting or consisting essentially of a eukaryotic promoter operably linked to a sequence encoding a protein of interest, and optionally a eukaryotic transcription termination sequence. Optionally the expression cassette may be a minimal expression cassette as defined above, i.e lacking one or more bacterial or vector sequences, typically selected from the group consisting of: (i) bacterial origins of replication; (ii) bacterial selection markers (typically antibiotic resistance genes) and (iii) unmethylated CpG motifs.

The DNA template may be provided in an amount sufficient for use in the process by any method known in the art. For example, the DNA template may be produced by the polymerase chain reaction (PCR). Where the DNA template is a dsDNA, it may be provided for the amplification step as denatured single strands by prior incubation at a temperature of at least 94 degrees centigrade. Thus, the process of the invention preferably comprises a step of denaturing a dsDNA template to provide single stranded DNA. Alternatively, the dsDNA template may be provided in double-stranded form. The whole or a selected portion of the DNA template may be amplified in the reaction.

The DNA template is contacted with at least one DNA polymerase under conditions promoting amplification of said template. Any DNA polymerase may be used. Any commercially available DNA polymerase is suitable for use in the process of the invention. Two, three, four, five or more different DNA polymerases may be used, for example one which provides a proof reading function and one or more others which do not. DNA polymerases having different mechanisms may be used e.g. strand displacement type polymerases and DNA polymerases replicating DNA by other methods. A suitable example of a DNA polymerase that does not have strand displacement activity is T4 DNA polymerase.

It is preferred that a DNA polymerase is highly stable, such that its activity is not substantially reduced by prolonged incubation under process conditions. Therefore, the enzyme preferably has a long half-life under a range of process conditions including but not limited to temperature and pH. It is also preferred that a DNA polymerase has one or more characteristics suitable for a manufacturing process. The DNA polymerase preferably has high fidelity, for example through having proof-reading activity. Furthermore, it is preferred that a DNA polymerase displays high processivity, high strand-displacement activity and a low Km for dNTPs and DNA. A DNA polymerase may be capable of using circular and/or linear DNA as template. The DNA polymerase may be capable of using dsDNA or ssdNA as a template. It is preferred that a DNA polymerase does not display non-specific exonuclease activity. Strand displacement-type polymerases are preferred.

In order to allow for amplification according to the invention, it is preferred that the DNA template is also contacted with one or more primers. The primers may be non-specific (i.e random in sequence) or may be specific for one or more sequences comprised within the DNA template. It is preferred that the primers are of random sequence so as to allow for non-specific initiation at any site on the DNA template. This allows for high efficiency of amplification through multiple initiation reactions from each template strand. Examples of random primers are hexamers, heptamers, octamers, nonamers, decamers or sequences greater in length, for example of 12, 15, 18, 20 or 30 nucleotides in length. A random primer may be of 6 to 30, 8 to 30 or 12 to 30 nucleotides in length. Random primers are typically provided as a mix of oligonucleotides which are representative of all potential combinations of e.g. hexamers, heptamers, octamers or nonamers in the DNA template.

In other embodiments, the primers are specific. This means they have a sequence which is complementary to a sequence in the DNA template from which initiation of amplification is desired. In this embodiment, a pair of primers may be used to specifically amplify a portion of the DNA template which is internal to the two primer binding sites. Primers may be unlabelled, or may comprise one or more labels, for example radionuclides or fluorescent dyes. Primers may also comprise chemically modified nucleotides. Primer lengths/sequences may typically be selected based on temperature considerations i.e as being able to bind to the template at the temperature used in the amplification step.

The contacting of the DNA template with the DNA polymerase and one or more primers takes place under conditions promoting annealing of primers to the DNA template. The conditions include the presence of single-stranded DNA allowing for hybridisation of the primers. The conditions also include a temperature and buffer allowing for annealing of the primer to the template.

Once the DNA template is contacted with the DNA polymerase and one or more primers, there is then a step of incubation under conditions promoting amplification of said template.

In addition to the amplification step, the process of the invention also comprises a processing step for production of closed linear DNA. Amplified DNA is contacted with at least one protelomerase under conditions promoting production of closed linear DNA. This simple processing step based on protelomerase is advantageous over other methods used for production of closed linear DNA molecules. The amplification and processing steps can be carried out simultaneously or concurrently. However, preferably, the amplification and processing steps are carried out sequentially with the processing step being carried out subsequent to the amplification step (i.e on amplified DNA).

The process of the invention is carried out in an in vitro cell-free environment. Thus, the process is carried out in the absence of a host cell and typically comprises use of purified enzymatic components. Accordingly, the amplification of a template DNA and processing by protelomerase is typically carried out by contacting the reaction components in solution in a suitable container. Optionally, particular components may be provided in immobilised form, such as attached to a solid support.

In certain embodiments, the template is an amplified linear open ended DNA, with blunt ends or overhangs and a synthesized hairpin molecule is ligated to one or both ends to form the closed ended linear DNA comprising at least one DD-ITR, or e.g. an ITR from FIG. 5. The template can be e.g. synthesized or PCR amplified. Unligated hairpins are purified away using means well known to those of skill in the art.

In one embodiment, the non-viral linear DNA is made within a cell.

It should be understood that the process of the invention may be carried out at any scale. However, it is preferred that the process is carried out to amplify DNA at a commercial or industrial scale i.e. generating amplified DNA in milligram or greater quantities. It is preferred that the process generates at least one milligram, at least 10 milligrams, at least 20 milligrams, at least 50 milligrams or at least 100 milligrams of amplified DNA. The final closed linear DNA product derived from the amplified DNA may also preferably be generated in milligram or greater quantities. It is preferred that the process generates at least one milligram, at least 2 milligrams, at least 5 milligrams, at least 10 milligrams, at least 20 milligrams, at least 50 milligrams, or at least 100 milligrams of closed linear DNA.

Formation of Mini-Circle DNA

An alternative method of generating covalently closed ends of the DD-ITR of the DNA construct is by formation of mini-circle DNA from plasmids. By way of non-limiting example, a parent nucleic that includes the DNA of interest (e.g., the DD-ITR and predetermined nucleic acid sequence in the form of an expression cassette) flanked by attB and attP sites of a unidirectional site specific recombinase is contacted with the unidirectional site specific recombinase that recognizes the flanking attB and attP sites under conditions sufficient for the unidirectional site specific recombinase to mediate a recombination event that produces a minicircle vector from the parent nucleic acid. By “flanked” is meant that the expression cassette and other sequence of interest that is to be present in the product minicircle vector, has an att site, e.g., attB and attP, at either end, such that the parent nucleic acid is described by the formula:
-------att(P or B) expression cassette-att(P or B)-------

The order of the att sites does not generally matter. The att sites are substrate sites for the unidirectional site specific recombinase, and are typically referred to as attB or attP sites by those of skill in the art. Sites of interest include, but are not limited to, the att sites recognized by the specific integrase recombinases above, as well as mutants thereof.

The parent nucleic acid may be present as a variety of different forms, depending at least in part on whether the production method is an in vitro or in vivo method. As such, the parent nucleic acid may be a linear double stranded nucleic acid, a closed circular nucleic acid (such as a bacterial plasmid suitable for use in replication), integrated into genomic DNA, and the like.

The method may be practiced in vitro or in vivo, e.g., inside of a cell. Where the method is practiced in vitro, all necessary reagents, e.g., parent nucleic acid, site specific integrase, etc., are combined into a reaction mixture and maintained under sufficient conditions for a sufficient period of time for the site specific recombinase mediated production of the desired product minicircle vectors to occur. Typically, for in vitro reactions, the reaction mixture is maintained at a temperature of between about 20 and 40° C.

In certain embodiments, the method is an in vitro method in that the recombinase mediated production of the desired product minicircle vector occurs inside of a cell in culture. Examples of such embodiments includes those embodiments where the parent nucleic acid is a plasmid that replicated in a bacterial host to produce large copy numbers of the parent nucleic acid prior to the recombinase mediated vector production step.

In the above in vivo embodiments, the first step may generally be to first prepare a host cell that includes large numbers of the parent nucleic acid. This may conveniently be done by transforming a host cell, e.g., E. coli., with a plasmid that will serve as the parent nucleic acid. The resultant transformed host cell is then maintained under conditions sufficient for the host cell to produce large copy numbers of the parent nucleic acid, as described above.

Upon provision of the host cell having sufficient copy numbers of the parent nucleic acid (e.g., plasmid), the unidirectional site-specific recombinase activity (i.e., that mediates production of the desired vector from the parent nucleic acid) is then produced in the host cell. The desired recombinase activity may be produced in the cell using any convenient protocol. In certain embodiments, the recombinase or a nucleic acid coding sequence therefore may be introduced into the host cell, e.g., as described above. Alternatively, the coding sequence for the recombinase may already be present in the host cell but not expressed, e.g., because it is under the control of an inducible promoter. In these embodiments, the inducible coding sequence may be present on the parent nucleic acid, present on another episomal nucleic acid, or even integrated into the host's genomic DNA. Representative inducible promoters of interest that may be operationally linked to the recombinase coding sequence include, but are not limited to: aracBAD promoter, the lambda pL promoter, and the like. In these embodiments, the step of providing the desired recombinase activity in the host cell includes inducing the inducible promoter to cause expression of the desired recombinase.

Following production of the desired recombinase activity in the host cell, the resultant host cell is then maintained under conditions and for a period of time sufficient for the recombinase activity to mediate production of the desired minicircle vectors from the parent nucleic acids. Typically, the host cell is maintained at a temperature of between about 20 and 40° C.

Following recombinase mediated production of the minicircle vectors from the parent nucleic acids, as described above, the product minicircles may then be separated from the remainder of their “synthesis” environment (e.g., reaction mixture, host cell, etc.) as desired. Any convenient protocol for separating the product minicircles may be employed. Representative protocols are described in U.S. Pat. Nos. 8,828,726, and 7,897,380.

Manufacture of Circular Nucleic Acids

Methods for manufacturing circular nucleic acids as described herein are further described in, e.g., U.S. Provisional Application No. 62/864,689; the contents of which are incorporated herein by reference in its entirety.

One aspect of the invention described herein provides a method of manufacturing circular nucleic acid vectors containing a transgene comprising: (a) contacting a host system with a template, wherein the template comprises at least one flanking cleavage sites, and (i) at least one phage origin of replication (ORI); (ii) at least one Terminal Repeat (TR), and; (iii) a promoter sequence operatively linked to a transgene, wherein at least one TR is an adeno associated virus (AAV) Double D ITR (DD-ITR); (b) incubating the host system for a time sufficient for replication to occur resulting in circular nucleic acid production; and (c) recovering the circular nucleic acid production, wherein the circular nucleic acid self-anneals.

Another aspect of the invention described herein provides a method of manufacturing circular nucleic acid vectors containing a transgene comprising: (a) transforming a host system with a plasmid template, wherein the plasmid template comprises: (i) a phage origin of replication (ORI); (ii) a truncated phage ORI (e.g., ORIΔ29); (iii) at least one Terminal Repeat (TR), and; (iv) a promoter sequence operatively linked to a transgene, wherein the plasmid template comprises, in the 5′ to 3′ direction, the sense sequence and the anti-sense sequence separated by a hairpin sequence that allows for annealing of the sense and anti-sense strand, and wherein at least one TR is an AAV Double D ITR (DD-ITR); (b) incubating the host system for a time sufficient for replication to occur resulting in circular nucleic acid production; and (c) recovering the circular nucleic acid production, wherein the circular nucleic acid self-anneals.

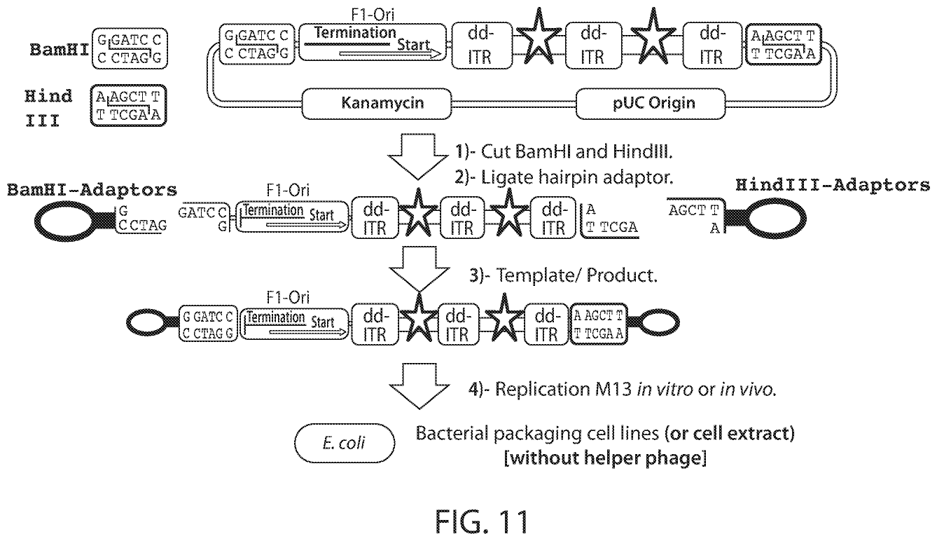

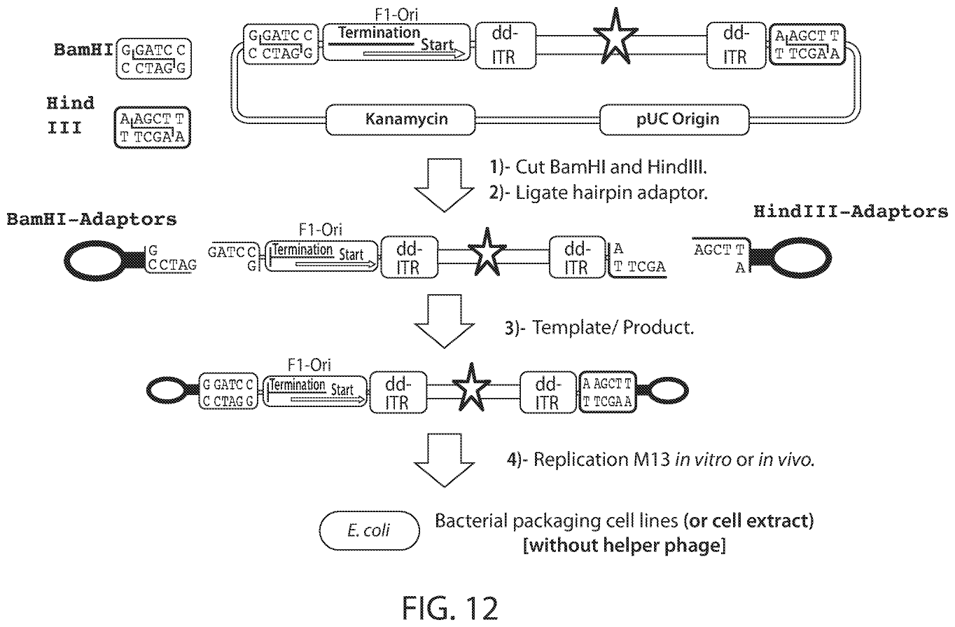

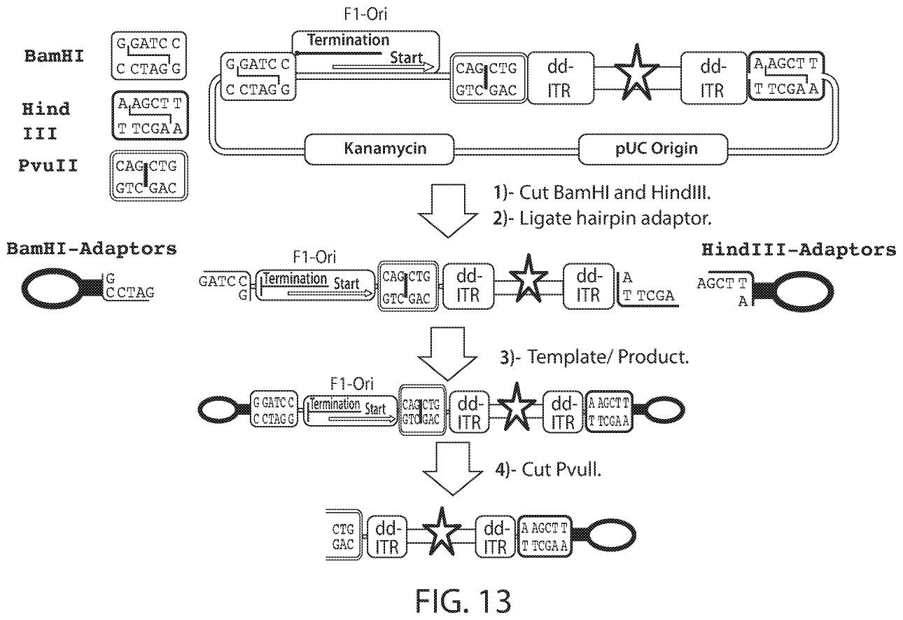

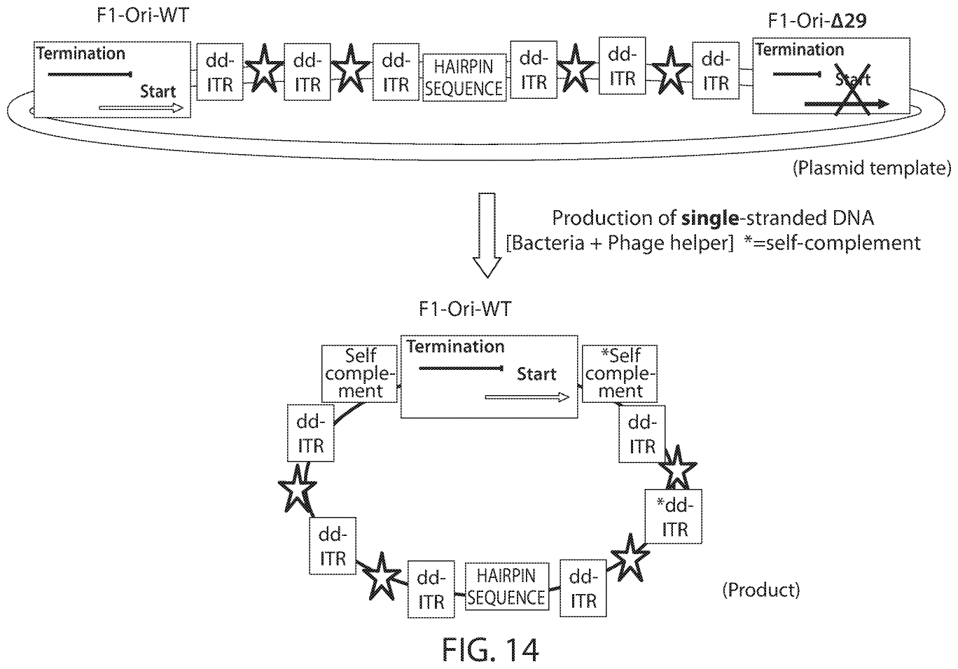

In one embodiment, a template used to produce circular nucleic acids is generated by cutting double-stranded plasmid DNA which comprises the components of the template (for example, see FIGS. 11-13) with a nuclease that specifically targets the cleavage site present on the plasmid, e.g., a restriction enzyme. In an alternative embodiment, double-stranded plasmid template can be used to produce circular nucleic acids (e.g., FIG. 14). A plasmid comprising components of a template, or a plasmid template described herein can be generated using standard cloning techniques known in the art. Cutting of the cleavage sites excises the plasmid fragment, i.e., a single-stranded linear DNA found between the two cleavage sites. In one embodiment, the plasmid fragment is than annealed to adaptor proteins at the cut ends. An adaptor sequence, e.g., having a complementary sequence to the cleavage site, would be capable of annealing to the cleavage site using standard techniques known in the art, for example, via ligation. Annealing the adaptor sequences to the ends of the plasmid fragment circularizes the DNA, creating a closed-end DNA structure, referred to herein as a template.

A template can be replicated in vitro or in vivo in a host system. For example, in E. coli cells using standard methods, e.g., as described in Shepherd, et al. Scientific Reports 9, Article number: 6121 (2019); cell extracts, e.g., E. coli cell extracts, as described in Wang, G., et al. Cell Research 7, 1-12(1997); or in a bacterial packaging cell line known in the art (the contents of these citations are incorporated herein by reference in their entireties). A bacterial packaging cell line can express M13-based helper plasmids, e.g., as described in Chastenn, L., et al. Nucleic Acids Res. 2006 December; 34(21): e145, the contents of which are incorporated herein by reference in its entirety. Alternatively, a template described herein need not undergo replication, and can be used to directly contact a host system, for example, an in vitro cell line.

Use of a phage ORI described herein is advantageous as it does not necessarily require the presence of a helper phage to initiate replication, eliminating the likelihood of helper phage contamination in the replicate. Phage ORIs described herein independently initiate replication of single-stranded circle, i.e., circular nucleic acids. The phage ORI located on the template initiates the replication of a single-stranded, complementary circle DNA, referred to herein as circular nucleic acid. In one embodiment, the template is incubated in the host system for a time sufficient to replicate circular nucleic acid. In one embodiment, the phage ORI initiates replication without requiring any additional components, e.g., helper phage. In an alternative embodiment, phage ORI-initiated replication occurs in the presence of additional components, e.g., helper phage. Helper phage particles, for example, M13K07, provide the necessary gene products for particle formation when using phage vectors. Helper phage particles are further reviewed in, for example, in (2005) Helper Phage. In: Encyclopedic Reference of Genomics and Proteomics in Molecular Medicine. Springer, Berlin, Heidelberg; the contents of which are incorporated herein by reference in its entirety.

In one embodiment, the template is single-stranded and in vitro or in vivo replication of the template generates single-stranded circular nucleic acids. The single-stranded circular DNA can self-anneal, for example, at the transgene sequence, and become double stranded.

When an ORI is present on both sides of the plasmid template, for example, a plasmid template having an F1 ORI and a ORIΔ29 flanking the other elements of the template (see, e.g., FIG. 14), the single-stranded circular nucleic acid contains a self-complementary transgene, e.g., a therapeutic transgene. In one embodiment, the single-stranded circular nucleic acid contains the sense sequence of the transgene and the anti-sense sequence of the transgene on one strand. In one embodiment, the sense and the anti-sense sequences are separated by a linker, e.g., a Holliday linker or a defective ITR, that allows the strand to bend and binding of the sense and antisense sequences to occur. It is specifically contemplated herein that the linker can be any sequence that allows for the bending of the strand that facilitates the binding of the sense and anti-sense sequences of the transgene. In one embodiment, the single-stranded circular nucleic acids further comprise a complement region and self-complement region flanking the ORI. See, e.g., FIG. 14.

Circular nucleic acids are released (i.e., set free) from the host system using standard techniques known for a specific host system, such as mechanical-mediated release (sonication) or chemical-mediated release (detergents). Following release, circular nucleic acids are recovered using standard methods, for example, via purification using column chromatography.

A circular nucleic acid generated herein can be closed-ended, open-ended, or both open-ended and closed ended. In one embodiment, the circular nucleic acid is closed-ended. A closed-ended DNA vector can have any configuration, for example, doggie bone, dumbbell, circular, closed-ended linear duplex, etc.

A circular nucleic acid replicate generated using methods described herein can be used for delivery of the transgene it expresses, or to generate more circular nucleic acids, e.g., via additional in vitro or in vivo replication. Circular nucleic acids manufactured using methods described herein can be used in the production of recombinant vectors, e.g., a recombinant viral vector.

Various additional aspects described herein provide vectors manufactured using any of the methods described herein.

A host system used for replication of the circular nucleic acid can be, e.g., an in vitro or in vivo host system. In one embodiment, the host system can be a host cell, such as an insect cell, a mammalian cell, a virus, a bacterial packaging cell, or a cell free system. A host system for manufacturing an AAV vector can further comprise a baculovirus expression system, for example, if the host system is an insect cell, e.g., Sf9, Sf21, Hi-5, and S2 insect cell lines. Baculovirus expression systems are further described in, e.g., U.S. Pat. Nos. 6,919,085B2; 6,225,060B1; 5,194,376A; the contents of each are incorporated herein by reference in their entireties. In a cell-free system, vectors can be synthesized and assembled in an in vitro system. In one embodiment, the cell-free system comprises helper phage particles.

The elements located on the circular nucleic acid, or template or plasmid template it is produced from, are not limited with respect to their location on the circular nucleic acid, or template or plasmid template from 5′ to 3′. For example, an ORI can be located upstream of a dd-ITR, or downstream of a dd-ITR, or both up and downstream of an dd-ITR. In another example, the ORI is flanked by the dd-ITRs and upstream of the promoter sequence operably linked to a transgene.

In one embodiment, the template contains an F1 ORI and has the sequence of SEQ ID NO: 37.

(SEQ ID NO: 37)
ACGCGCCCTGTAGCGGCGCATTAAGCGCGGCGGGTGTGGTGGTTACGCGC
AGCGTGACCGCTACACTTGCCAGCGCCCTAGCGCCCGCTCCTTTCGCTTT
CTTCCCTTCCTTTCTCGCCACGTTCGCCGGCTTTCCCCGTCAAGCTCTAA
ATCGGGGGCTCCCTTTAGGGTTCCGATTTAGTGCTTTACGGCACCTCGAC
CCCAAAAAACTTGATTAGGGTGATGGTTCACGTAGTGGGCCATCGCCCTG
ATAGACGGTTTTTCGCCCTTTGACGTTGGAGTCCACGTTCTTTAATAGTG
GACTCTTGTTCCAAACTGGAACAACACTCAACCCTATCTCGGTCTATTCT
TTTGATTTATAAGGGATTTTGCCGATTTCGGCCTATTGGTTAAAAAATGA
GCTGATTTAACAAAAATTTAACGCGAATTA

In another embodiment, the ORI is derived from M13 and facilitates M13 helper-dependent replication of the template. M13 ORI has the nucleotide sequence of SEQ ID NO: 38.

(SEQ ID NO: 38)
ACGCGCCCTGTAGCGGCGCATTAAGCGCGGCGGGTGTGGTGGTTACGCGCA
GCGTGACCGCTACACTTGCCAGCGCCCTAGCGCCCGCTCCTTTCGCTTTCT
TCCCTTCCTTTCTCGCCACGTTCGCCGGCTTTCCCCGTCAAGCTCTAAATC
GGGGGCTCCCTTTAGGGTTCCGATTTAGTGCTTTACGGCACCTCGACCCCA
AAAAACTTGATTTGGGTGATGGTTCACGTAGTGGGCCATCGCCCTGATAGA
CGGTTTTTCGCCCTTTGACGTTGGAGTCCACGTTCTTTAATAGTGGACTCT
TGTTCCAAACTGGAACAACACTCAACCCTATCTCGGGCTATTCTTTTGATT
TATAAGGGATTTTGCCGATTTCGG

In one embodiment, the at least one ORI includes a second ORI that is mutated as compared to a wild-type ORI. A mutated ORI can comprise single nucleotide mutations, e.g., nucleotide deletion, insertion, or substitutions) or can be truncated to lack at least a portion (e.g., at least five nucleotides) of the wild-type ORI sequence. In one embodiment, the mutant ORI is a mutant F1 ORI, F1-ORIΔ29. Mutant ORIΔ29 is a truncated F1 ORI that lacks the capacity to initiate replication. ORIΔ29 is further reviewed in, e.g., Specthrie, L, et al. Journal of Mol Biol. V. 228(3), 1992. In one embodiment, ORIΔ29 has the nucleotide sequence of SEQ ID NO: 39.

(SEQ ID NO: 39)
ACGCGCCCTGTAGCGGCGCATTAAGCGCGGCGGGTGTGGTGGTTACGCG
CAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCT
CGACCCCAAAAAACTTGATTAGGGTGATGGTTCACGTAGTGGGCCATCG
CCCTGATAGACGGTTTTTCGCCCTTTGACGTTGGAGTCCACGTTCTTTA
ATAGTGGACTCTTGTTCCAAACTGGTTTAACACTC

In one embodiment, the mutant ORI is a mutant M13 ORI, M13-ORIΔ29. Mutant ORIΔ29 is a truncated M13 ORI that lacks the capacity to initiate replication. In one embodiment, ORIΔ29 has the nucleotide sequence of SEQ ID NO: 40.

(SEQ ID NO: 40)
ACGCGCCCTGTAGCGGCGCATTAAGCGCGGCGGGTGTGGTGGTTACGCGC
AGCGTGACCGCTACACTTGCCAGCGCCCTAGCGCCCGCTCCTTTCGCTTT
CTTCCCTTCCTTTCTCGCCACGTTCGCCGGCTTTCCCCGTCAAGCTCTAA
ATCGGGGGCTCCCTTTAGGGTTCCGATTTAGTGCTTTACGGCACCTCGAC
CCCAAAAAACTTGATTTGGGTGATGGTTCACGTAGTGGGCCATCGCCCTG
ATAGACGGTTTTTCGCCCTTTGACGTTGGAGTCCACGTTCTTTAATAGTG
GACTCTTGTTCCAAACTGGTTTAACACTC

Circular nucleic acids described herein do not comprise other types or species of ORI, for example, the vector does not comprise bacterial or mammalian ORI.

A cleavage site is a nucleotide sequence in which the phosphodiester backbone is selectively broken. For example, a nucleotide sequence recognized by a nuclease is a cleavage site because the enzyme will cut the phosphodiester backbone at selective sites within the sequence. Such cleavage sites may be single or double-stranded, depending on the endonuclease. Also included are chemical cleavage sites such as pyrimidine and purine cleavage reactions performed in Maxam and Gilbert sequencing, or cleavage through chemical methods such as oxidation as described in U.S. Pat. No. 4,795,700, which is incorporated herein by reference.

In one embodiment, the template further comprises at least a second cleavage, and within the sites are the additional elements contained on the template, e.g., at least on ORI, at least one TR (at least one of which is a DD-ITR), and promotor operatively linked to a therapeutic transgene, such that the at least two cleavage sites flank these elements. In one embodiment, a third, unique cleavage site is located immediately downstream of the ORI.

In one embodiment, the cleavage site is cut by a nuclease. As used herein, the term “nuclease” refers to molecules which possesses activity for DNA cleavage. In one embodiment, the nuclease is protelomerase and the cleavage site is a protelomerase target sequence, e.g., the TelN recognition site. In one embodiment, the nuclease is a restriction endonuclease and the cleavage site is a recognition site for the endonuclease (i.e., a restriction site). Restriction endonucleases are hydrolytic enzymes capable of catalyzing site-specific cleavage of DNA molecules. The locus of restriction endonuclease action is determined by the existence of a specific nucleotide sequence. Such a sequence is termed the recognition site for the restriction endonuclease. When at least two cleavage sites are present, the at least two site can be the same cleavage sites or difference cleave sites.

In one embodiment, a restriction site in the template is an uncommon restriction site, i.e., it is not commonly found in the sequence of transgenes. Exemplary uncommon restriction sites include a mirror-like palindrome restriction site, or an 8 base pair restriction site. In one embodiment, the restriction site used in the template is not found in the transgene, i.e., therapeutic transgene, of the invention.

An adaptor sequence is a short, synthesized, single-stranded or double-stranded oligonucleotide that can be ligated to the ends of other DNA or RNA molecules. In one embodiment, an adaptor sequences described herein are single-stranded and close the DNA end it is ligated to, e.g., through a hairpin loop. Adaptor sequences are added to one or both ends of the cut plasmid fragment as a means of circularizing the DNA. In one embodiment, the adaptor sequence is ligated to the plasmid fragment and directs the closure at the end of the cleaved DNA which it is ligated to (see e.g., FIGS. 11-13). Exemplary adaptor proteins that can be used to close the DNA end include hairpin loops further described, e.g., in U.S. Application No. 2009/0098612; and U.S. Pat. Nos. 6,369,038; 6,451,563; 6,849,725; the contents of which are incorporated herein by reference in their entireties. It is envisioned that any sequence that can circularize the DNA when added to the cut end of a plasmid fragment excised from a plasmid can be an adaptor sequence.

By way of example, a hairpin loop adaptor sequence having the sequence of SEQ ID NO. 41 (CCATTCTGTTCCGCATGATTCCTCTGCGGAACAGAATGG (SEQ ID NO: 41) can further comprise an Sfi1 restriction site sequence (e.g., GGCC GGCC; SEQ ID NO: 42). The adaptor sequence having a Sfi1 restriction site sequence can be digested with the restriction enzyme, Sfi1, for a time sufficient to cut the restriction site. This would create “sticky ends” on the adaptor sequence that can be used to hybridize the adaptor protein to a plasmid fragment excised via the Sfi1 restriction enzyme.

Therapeutic Nucleic Acids

The DNA constructs of the present invention are useful for the delivery of nucleic acids to cells in vitro, ex vivo, and in vivo. In particular, the DNA constructs can be advantageously employed to deliver or transfer nucleic acids to animal, including mammalian, cells.

Any nucleic acid sequence(s) of interest may be delivered in the DNA constructs of the present invention. Nucleic acids of interest include nucleic acids encoding polypeptides, including therapeutic (e.g., for medical or veterinary uses), immunogenic (e.g., for vaccines), or diagnostic polypeptides.

Therapeutic polypeptides include, but are not limited to, cystic fibrosis transmembrane regulator protein (CFTR), dystrophin (including mini- and micro-dystrophins (see, e.g, Vincent et al., (1993) Nature Genetics 5:130; U.S. Patent Publication No. 2003/017131; International publication WO/2008/088895, Wang et al., Proc. Natl. Acad. Sci. USA 97:13714-13719 (2000); and Gregorevic et al., Mol. Ther. 16:657-64 (2008)), myostatin propeptide, follistatin, activin type II soluble receptor, IGF-1, anti-inflammatory polypeptides such as the Ikappa B dominant mutant, sarcospan, utrophin (Tinsley et al., (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, beta-globin, alpha-globin, spectrin, alphas-antitrypsin, adenosine deaminase, hypoxanthine guanine phosphoribosyl transferase, beta-glucocerebrosidase, sphingomyelinase, lysosomal hexosaminidase A, branched-chain keto acid dehydrogenase, RP65 protein, cytokines (e.g., alpha-interferon, beta-interferon, interferon-gamma., interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor-3 and -4, brain-derived neurotrophic factor, bone morphogenic proteins [including RANKL and VEGF], glial derived growth factor, transforming growth factor-.alpha. and -.beta., and the like), lysosomal acid .alpha.-glucosidase, .alpha.-galactosidase A, receptors (e.g., the tumor necrosis growth factory soluble receptor), S100A1, parvalbumin, adenylyl cyclase type 6, a molecule that effects G-protein coupled receptor kinase type 2 knockdown such as a truncated constitutively active bARKet, anti-inflammatory factors such as TRAP, anti-myostatin proteins, aspartoacylase, and monoclonal antibodies (including single chain monoclonal antibodies; an exemplary Mab is the Herceptin® Mab). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. Parvovirus vectors can also be used to deliver monoclonal antibodies and antibody fragments, for example, an antibody or antibody fragment directed against myostatin (see, e.g., Fang et al., Nature Biotechnol. 23:584-590 (2005)).

Nucleic acid sequences encoding polypeptides include those encoding reporter polypeptides (e.g., an enzyme). Reporter polypeptides are known in the art and include, but are not limited to, Green Fluorescent Protein, beta-galactosidase, alkaline phosphatase, luciferase, and chloramphenicol acetyltransferase gene.

Alternatively, in particular embodiments of this invention, the nucleic acid may encode an antisense nucleic acid, a ribozyme (e.g., as described in U.S. Pat. No. 5,877,022), RNAs that effect spliceosome-mediated trans-splicing (see, Puttaraju et al., (1999) Nature Biotech 17:246; U.S. Pat. Nos. 6,013,487; 6,083,702), interfering RNAs (RNAi) including siRNA, shRNA or miRNA that mediate gene silencing (see, Sharp et al., (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10:132-142 (2008) and Li et al., Acta Pharmacol Sin. 26:51-55 (2005)); phospholamban inhibitory or dominant-negative molecules such as phospholamban S16E (e.g., to treat cardiovascular disease, see, e.g., Hoshijima et al. Nat. Med. 8:864-871 (2002)), RNAi to adenosine kinase (e.g., for epilepsy), RNAi to a sarcoglycan (e.g., alpha., beta., gamma), RNAi against myostatin, myostatin propeptide, follistatin, or activin type II soluble receptor, RNAi against anti-inflammatory polypeptides such as the Ikappa B dominant mutant, and RNAi directed against pathogenic organisms and viruses (e.g., hepatitis B virus, human immunodeficiency virus, CMV, herpes simplex virus, human papilloma virus, etc.).

Alternatively, in particular embodiments of this invention, the nucleic acid may encode protein phosphatase inhibitor I (I-1), serca2a, zinc finger proteins that regulate the phospholamban gene, Barkct, beta 2-adrenergic receptor, beta 2-adrenergic receptor kinase (BARK), phosphoinositide-3 kinase (PI3 kinase), a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, enos, inos, or bone morphogenie proteins (including BNP 2, 7, etc., RANKL and/or VEGF).

The DNA constructs may also comprise a nucleic acid that shares homology with and recombines with a locus on a host chromosome. This approach can be utilized, for example, to correct a genetic defect in the host cell.

As a further alternative, the nucleic acid can encode any polypeptide that is desirably produced in a cell in vitro, ex vivo, or in vivo. For example, the DNA construct may be introduced into cultured cells and the expressed gene product isolated therefrom.

Regulatory Elements

The nucleic acid may be in the context of an expression cassette. It will be understood by those skilled in the art that the nucleic acid(s) of interest can be operably associated with appropriate control sequences. For example, the nucleic acid can be operably associated with expression control elements, such as transcription/translation control signals, origins of replication, polyadenylation signals, internal ribosome entry sites (IRES), promoters, and/or enhancers, and the like. In preferred embodiments of the invention, the expression cassette comprises a eukaryotic promoter operably linked to a coding sequence of interest, and optionally a eukaryotic transcription termination sequence. Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host/target cell into which the transcriptional initiation region is introduced.

In particular embodiments, the promoter/enhancer elements can be native to the target cell or subject to be treated. In representative embodiments, the promoters/enhancer element can be native to the nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the heterologous nucleic acid sequence(s). Inducible promoters/enhancer elements for gene delivery can be tissue-specific or -preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

Preparation of Viral Particles

Aspects of the invention relate to covalently closed non-viral linear DNA construct (sometimes closed linear DNA) to produce recombinant viral particles for delivery of construct DNA as a single stranded recombinant viral genome. This involves adding the closed linear DNA construct comprising at least two DD-ITRs (e.g., see FIG. 1) into a host cell along with vectors or plasmids capable of (1) expressing AAV Rep proteins necessary to nick at least one of the DD-ITRs, and (2) expressing parvovirus viral capsid proteins, e.g., a dependovirus such as AAV, necessary to form a virion (viral particle) that can encapsidate the ITRs and the intervening DNA sequence. In the host cell the DD-ITRs resolve and replication is initiated from the self-primed ITR in the presence of Rep to produce vector genomes that can be packaged. Preferably, the intervening DNA sequence contains a transgene sequence such as a therapeutic gene sequence. The particular Rep protein that nicks, e.g., an AAV ITR is typically from the same serotype (for example, AAV2 ITR with AAV2Rep78) although any serotypes can be used as long as they retain at least 25% . . . 35% . . . 50% . . . 65% . . . 75% . . . 85% . . . 90% . . . 95% . . . 98% . . . 100% or any intervening percent or greater percent nicking efficacy as compared to the situation when the AAV ITR and the AAV Rep protein are from the same serotype. One can use this method to prepare large amounts of viral particles containing the ITRs and their intervening DNA sequence, e.g. transgene. Using the closed linear vector containing the DD-ITR is more efficient, i.e. it provides a higher yield of packaged genomes, infectious viral particles as compared to a closed linear vector not having the Double D, and the viral particles do not have any contaminating plasmid DNA.

With this method one can produce any known AAV particles such as those containing synthetic ITRs, or self-complementary dimeric AAV sequences (sc dimer) etc. The skilled artisan knows that the typical AAV particle cannot package more than about 5,000 nts and thus care is taken in designing the initial non-viral closed linear DNA construct to be a packagable size. In one embodiment, when packaged self-complementary is desired the initial non-viral closed linear DNA construct rAAV genome vector template, for example can have a Rep nick defective ITR or a hairpin sequence. See for example, U.S. Pat. Nos. 7,465,583, 7,790,154, 8,361,457, 8,784,799 incorporated by reference in their entirety. For example, in one embodiment, the vector template can be an approximately ½ size genome template (e.g. around ½ size of a wild type AAV genome) and one of the DD-ITRs is a Rep-nick incompetent ITR. The rescued and replicated rAAV genome would then be full length genome size and packageable. In an alternative embodiment, the closed linear DNA construct rAAV genome vector template comprises a nick defective ITR, or e.g. a hairpin sequence, that is placed between to two Rep-nick competent DD-ITRs and the nick defective ITR, or hairpin sequence is further in between the sense and anti-sense sequences of the transgene, and the genome template is approximately full size, e.g. around 5,000 nts or wtAAV size, e.g. less than 5000 nts . . . 4800 nts . . . 4500 nts. . . . In either case, after replication of the closed linear rAAV vector genome template, the sense and anti-sense sequences are on a single strand of DNA in between ITRs and this vector genome can be packaged within the rAAV virion.

The host cell can already be expressing the Rep and viral capsid proteins or one can co-transfect the cells at the same or about the same time by standard means. Any of the known cell-types used in the art can be used in this process. For example, one can use an insect cell such as baculovirus. Methods for production using mammalian suspension cells are for example described in U.S. Pat. No. 9,411,206, incorporated herein by reference in its entirety.

Pharmaceutical compositions comprising the vectors described herein are also provided.

In embodiments of the methods and vectors described herein, the ITR sequence is flanked on either side by complementary sequences D and D′ (e.g., is a DD-ITR).

In embodiments of the methods and vectors described herein, the D regions contain a nicking site.

The ITR sequences of a nucleic acid or nucleic acid vector described herein can be derived from any parviovirus or any AAV serotype (e.g., 1, 2, 3, 4, 5, 6, 7, 8, 9, or 10) or can be derived from more than one serotype. In some embodiments of the nucleic acid or nucleic acid vector provided herein, the ITR sequences are derived from AAV2. In some embodiments of the nucleic acid or nucleic acid vector provided herein, the ITR sequences are derived from an autonomus parvovirus. ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. Kessler PD, Podsakoff G M, Chen X, McQuiston S A, Colosi P C, Matelis L A, Kurtzman G J, Byrne B J. Proc Natl Acad Sci USA. 1996 Nov. 26; 93(24): 14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference). In some embodiments, the expression construct is no more than 5 kilobases, no more than 4 kilobases, or no more than 3 kilobases in size. In some embodiments, the expression construct is between 4 and 7 kilobases in size. In one embodiment, the replicated genome obtained from rescue and replicaiton of the rAAV genome from the closed linear DNA vectors described herein is enccapsidated in a parvovirus particle such as a dependovirus particle. For instance an AAV particle. The rAAV particle may be of any AAV serotype (e.g., 1, 2, 3, 4, 5, 6, 7, 8, 9, or 10), including any derivative (including non-naturally occurring variants of a serotype) or chimeric or pseudotype. The replicated genome is a packageable size.

Non-limiting examples of derivatives and chimerics include AAV2-AAV3, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShHIO, AAV2 (Y->F), AAV8 (Y733F), AAV2.15, AAV2.4, AAVM41, and AAVr3.45 (see, e.g., Mol Ther. 2012 April; 20(4):699-708. doi: 10.1038/mt.2011.287. Epub 2012 Jan. 24. The AAV vector toolkit: poised at the clinical crossroads. Asokan Al, Schaffer D V, Samulski R J.).

In some embodiments, the rAAV particle comprises a capsid that includes modified capsid proteins (e.g., capsid proteins comprising a modified VP3, VP1, or VP2). Methods of producing modified capsid proteins are known in the art (see, e.g., U.S. Patent Publication Number US20130310443, which is incorporated herein by reference in its entirety). In some embodiments, the rAAV particle comprises a modified capsid protein comprising at least one non-native amino acid substitution at a position that corresponds to a surface-exposed amino acid in a wild-type capsid protein (e.g., wild-type AAV2 capsid protein, such as SEQ ID NO: 15, or other wild-type AAV capsid protein). In some embodiments, the rAAV particle comprises a modified capsid protein comprising a non-tyrosine amino acid (e.g., a phenylalanine) at a position that corresponds to a surface-exposed tyrosine amino acid in a wild-type capsid protein, a non-threonine amino acid (e.g., a valine) at a position that corresponds to a surface-exposed threonine amino acid in the wild-type capsid protein, a non-lysine amino acid (e.g., a glutamic acid) at a position that corresponds to a surface-exposed lysine amino acid in the wild-type capsid protein, a non-serine amino acid (e.g., a valine) at a position that corresponds to a surface-exposed serine amino acid in the wild-type capsid protein, or a combination thereof.

An exemplary, non-limiting wild-type AAV2 capsid protein sequence is provided below (SEQ ID NO: 15).

1 MAADGYLPDW LEDTLSEGIR QWWKLKPGPP PPKPAERHKD DSRGLVLPGY 51 KYLGPFNGLD
KGEPVNEADA AALEHDKAYD RQLDSGDNPY LKYNHADAEF 101 QERLKEDTSF GGNLGRAVFQ
AKKRVLEPLG LVEEPVKTAP GKKRPVEHSP 151 VEPDSSSGTG KAGQQPARKR LNFGQTGDAD
SVPDPQPLGQ PPAAPSGLGT 201 NTM ATGS GAP MADNNEGADG VGNSSGNWHC DSTWMGDRVI
TTSTRTWALP 251 TYNNHLYKQI SSQSGASNDN HYFGYSTPWG YFDFNRFHCH FSPRDWQRLI 301
NNNWGFRPKR LNFKLFNIQV KEVTQNDGTT TIANNLTSTV QVFTDSEYQL 351 PYVLGS AHQG
CLPPFPADVF MVPQYGYLTL NNGSQAVGRS SFYCLEYFPS 401 QMLRTGNNFT FSYTFEDVPF
HSSYAHSQSL DRLMNPLIDQ YLYYLSRTNT 451 PSGTTTQSRL QFSQAGASDI RDQSRNWLPG
PCYRQQRVSK TS ADNNNSEY 501 SWTGATKYHL NGRDSLVNPG PAMASHKDDE EKFFPQSGVL
IFGKQGSEKT 551 NVDIEKVMIT DEEEIRTTNP VATEQYGS VS TNLQRGNRQA ATADVNTQGV 601
LPGMVWQDRD VYLQGPIWAK IPHTDGHFHP SPLMGGFGLK HPPPQILIKN 651 TPVPANPSTT FS
AAKFASFI TQYSTGQVS V EIEWELQKEN SKRWNPEIQY 701 TSNYNKSVNV DFTVDTNGVY
SEPRPIGTRY LTRNL

Methods of producing rAAV particles and nucleic acid vectors are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, the nucleic acid vector (e.g., the closed linear vector comprising DD-ITR and heterologus gene) may be combined with one or more helper plasmids, e.g., that contain a rep gene (e.g., encoding Rep78, Rep68, Rep52 and Rep40) and a cap gene (encoding VP1, VP2, and VP3), and transfected into a producer cell line such that the rAAV vector genome can be replicated, packaged and subsequently purified.

Any suitable cell known in the art may be employed. In particular embodiments, the cell is a mammalian cell. As another option, the cell can be a trans-complementing packaging cell line that provides functions deleted from a replication-defective helper virus, e.g., 293 cells or other Ela trans-complementing cells.

The AAV replication and capsid sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the Ela or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., are stably integrated into the cell as extra-chromosomal elements, designated as an “EBV based nuclear episome,” see Margolski, (1992) Curr. Top. Microbiol. Immun. 158:67).

Modified Capsids:

Modified capsid proteins of the invention can be produced by modifying the capsid protein of any AAV now known or later discovered. Further, the AAV capsid protein that is to be modified can be a naturally occurring AAV capsid protein (e.g., an AAV2, AAV3a or 3b, AAV4, AAV5, AAV8, AAV9, AAV10 or AAV11 capsid protein or any of the AAV shown in Table 1) but is not so limited. Those skilled in the art will understand that a variety of manipulations to the AAV capsid proteins are known in the art and the invention is not limited to modifications of naturally occurring AAV capsid proteins. For example, the capsid protein to be modified may already have alterations such as insertions, deletions or substitutions as compared with naturally occurring AAV (e.g., is derived from a naturally occurring AAV capsid protein, e.g., AAV2, AAV3a, AAV3b, AAV4, AAV5, AAV6, AAV7, AAV8, AAV9, AAV10 and/or AAV11 or any other AAV now known or later discovered). Such AAV capsid proteins are also within the scope of the present invention.

In one embodiment, the capsid protein can be modified such that it has a phenotype of decreased liver transduction and/or reduced glycan binding affinity as compared an unmodified capsid protein. It would be well known to one of skill in the art what the equivalent amino acids are in other AAV serotypes and the present invention encompasses such other AAV serotypes, comprising, consisting essentially of, or consisting of the mutation(s) of this invention at such equivalent amino acid positions, wherein said mutation(s) result in a phenotype of reduced liver transduction and/or reduced glycan binding affinity as compared to a control.

In particular embodiments, the capsid protein comprises 1, 2, 3, 4, 5, 6, 7, 8, 9, 10, 11, 12, 13, 14, 15, 16, 17, 18, 19 or 20, less than 20, less than 30, less than 40 less than 50, less than 60, or less than 70 amino acids alterations as compared with the native AAV capsid protein sequence. See U.S. Pat. No. 9,409,953, which is incorporated herein by reference.

To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production as described by Ferrari et al., (1997) Nature Med. 3:1295, and U.S. Pat. Nos. 6,040,183 and 6,093,570.

Further, the helper virus functions may be provided by a packaging cell with the helper sequences embedded in the chromosome or maintained as a stable extrachromosomal element. Generally, the helper viruses sequences cannot be packaged into AAV virions, e.g., are not flanked by TRs.

In some embodiments, the one or more helper plasmids includes a first helper plasmid comprising a rep gene and a cap gene and a second helper plasmid comprising other genes that assist in AAV production, such as a Ela gene, a Elb gene, a E4 gene, a E2a gene, and a VA gene. In some embodiments, the rep gene is a rep gene derived from AAV2 and the cap gene is derived from AAV5. Helper plasmids, and methods of making such plasmids, are known in the art and commercially available (see, e.g., pDM, pDG, pDPlrs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. Alternatively, in another example, Sf9-based producer stable cell lines are infected with a single recombinant baculovirus containing the nucleic acid vector. The rAAV particles can then be purified using any method known in the art or described herein, e.g., by iodixanol step gradient, CsCl gradient, chromatography, or polyethylene glycol (PEG) precipitation.

Various means of recombinant AAV production are well known to those of skill in the art and any of these methods can be used with the modification of substituting the vector template described herein with the covalently closed non-viral linear DNA construct comprising at least two DD-ITRs described herein.

The disclosure also contemplates host cells that comprise at least one of the disclosed rAAV particles, expression constructs, or nucleic acid vectors. Such host cells include mammalian host cells, with human host cells being preferred, and may be either isolated, in cell or tissue culture. In the case of genetically modified animal models (e.g., a mouse), the transformed host cells may be comprised within the body of a non-human animal itself.

In one embodiment, the rAAV using the closed linear or minicircle DNA, e.g. comprising the DD ITR, is produced in a cell line, e.g. the Pro10 cell line as described in U.S. Pat. No. 9,441,206, herein incorporated by reference in its entirety.

Delivery of DNA Construct to Target Cells

The DNA vector constructs either the viral particles or the naked DNA of the invention may be delivered to target cells by various available means in the art. Methods of delivery of nucleic acids include, without limitation infection by particles, lipofection, nucleofection, microinjection, biolistics, liposomes, immunoliposomes, polycation or lipid:nucleic acid conjugates, naked DNA, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Delivery can be to cells (e.g., in vitro or ex vivo administration) or target tissues (e.g., in vivo administration).

In one embodiment, the DNA construct described herein is administered to a cell by transfection. Transfection methods useful for the methods described herein include, but are not limited to, lipid-mediated transfection, cationic polymer-mediated transfection, or calcium phosphate precipitation. Transfection agents suitable for use with the invention include transfection agents that facilitate the introduction of RNA, DNA and proteins into cells. Exemplary transfection reagents include TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.).

In another embodiment, the DNA construct described herein is administered to a cell by electroporation (e.g., nucleofection). In some embodiments, the nucleic acids described herein are administered to a cell via microfluidics methods known to those of skill in the art.

Liposome-Mediated Delivery

In embodiments, the DNA construct is added to liposomes for delivery to a cell. Liposomes are vesicles that possess at least one lipid bilayer. Liposomes are typically used as carriers for drug/therapeutic delivery in the context of pharmaceutical development. Liposomes work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (di stearoyl-sn-glycero-phosphoethanolamine); DSPC (di stearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine). cholesteryl sulphate (CS), dipalmitoylphosphatidylglycerol (DPPG), DOPC (dioleoly-sn glycero-phosphatidylcholine) or any combination thereof.

In some aspects, the disclosure provides for a liposome formulation including phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation including a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation including a lipid containing a phosphatidylcholine functional group, cholesterol and a PEG-ylated lipid. In some aspects, the disclosure provides for a liposome formulation including a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g. cholesterol. In some aspects, the liposome formulation comprises DOPC/DEPC; and DOPE.

In some aspects, the disclosure provides for a liposome formulation further comprising one or more pharmaceutical excipients, e.g., sucrose and/or glycine.

In some aspects, the disclosure provides for a liposome formulation that is either unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder.

In some aspects, the disclosure provides for a liposome formulation that is made and loaded with the DNA construct obtained by the process of Example 1 or otherwise disclosed herein, by adding a weak base to a mixture having the isolated DNA construct outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g., polyphosphate or sucrose octasulfate.

In other aspects, the disclosure provides for a liposome formulation comprising phospholipids, lecithin, phosphatidylcholine and phosphatidylethanolamine.

In some aspects, the disclosure provides for lipid formulations, for example, lipid nanoparticle formulations, that are useful in delivering the DNA construct. For example, the lipid nanoparticle formulations described in WO2017/173054, the contents of which are incorporated herein by reference in its entirety, are contemplated for use with the methods and compositions described herein.

Pharmaceutical Compositions

The DNA vector constructs disclosed herein can be incorporated into pharmaceutical compositions suitable for administration to a subject for in vivo delivery to cells, tissues, or organs of the subject. Typically, the pharmaceutical composition includes the DNA construct disclosed herein and a pharmaceutically acceptable carrier. For example, the DNA construct can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high DNA construct concentration. Sterile injectable solutions can be prepared by incorporating the DNA construct compound in the required amount in an appropriate buffer with one or a combination of ingredients enumerated above, as required, followed by filtered sterilization including a DNA construct can be formulated to deliver a transgene in the nucleic acid to the cells of a recipient, resulting in the therapeutic expression of the nucleic acid therein. The composition can also include a pharmaceutically acceptable carrier.

The compositions and vectors provided herein can be used to deliver a predetermined DNA sequence (e.g., a transgene or donor sequence) for various purposes. In some embodiments, the DNA sequence encodes an RNA or protein that is intended to be used for research purposes, e.g., to create a somatic transgenic animal model harboring the transgene, e.g., to study the function of the protein to which the expressed protein or RNA interacts. In another example, the transgene encodes that is intended to be used to create an animal model of disease. In some embodiments, the transgene encodes one or more peptides, polypeptides, or proteins, which are useful for the treatment or prevention of disease states in a mammalian subject.

Pharmaceutical compositions for therapeutic purposes typically must be sterile and stable under the conditions of manufacture and storage. The composition can be formulated as a solution, microemulsion, dispersion, liposomes, or other ordered structure suitable to high DNA construct or virus particle concentration. Sterile injectable solutions can be prepared by incorporating the DNA construct compound in the required amount in an appropriate buffer with one or a combination of ingredients enumerated above, as required, followed by filtered sterilization.

Dosage, Administration and Efficacy

The methods comprise administering to the subject an effective amount of a composition comprising a DNA construct encoding a therapeutic protein or RNA or virus particle as described herein. As will be appreciated by a skilled practitioner, the term “effective amount” refers to the amount of the DNA construct composition administered that results in expression of the encoded protein or RNA in a “therapeutically effective amount” for the treatment of a disease.

The dosage ranges for the composition comprising a DNA construct depends upon the potency (e.g., efficiency of the promoter), and includes amounts large enough to produce the desired effect, e.g., expression of the desired protein or RNA, for treatment of a disease, e.g., cancer. The dosage should not be so large as to cause unacceptable adverse side effects. Generally, the dosage will vary with the particular characteristics of the DNA construct, expression efficiency and with the age, condition, and sex of the patient. The dosage can be determined by one of skill in the art. For example in mice, where non-viral DNA vector is intravenously administered in saline, in one embodiment, the amount of vector that is administered to a target cell can range from about 1 to 200 ug, or about 10 to 50 ug. One of skill in that adjusts the dose accordingly for administration to humans or larger animals. In certain embodiments the size of the vector ranges from about 0.5 to 100 kb, or from about 2 to 15 kb.

As used herein, the term “therapeutically effective amount” is an amount of an expressed therapeutic protein or RNA that is sufficient to produce a statistically significant, measurable change in expression of a disease biomarker or reduction in a given disease symptom (see “Efficacy Measurement” below). Such effective amounts can be gauged in clinical trials as well as animal studies for a given DNA construct or virus particle composition.

Agents useful in the methods and compositions described herein can be administered topically, intravenously (by bolus or continuous infusion), intracellular injection, intratissue injection, orally, by inhalation, intraperitoneally, intramuscularly, subcutaneously, intracavity, and can be delivered by peristaltic means, if desired, or by other means known by those skilled in the art. The agent can be administered systemically, if so desired.

The efficacy of a given treatment for a given disease, such as cancer (including, but not limited to, breast cancer, melanoma etc.) can be determined by the skilled clinician. However, a treatment is considered “effective treatment,” as the term is used herein, if any one or all of the signs or symptoms of the disease or disorder is/are altered in a beneficial manner, or other clinically accepted symptoms or markers of disease are improved, or ameliorated, e.g., by at least 10% following treatment with a DNA construct encoding a therapeutic protein or RNA. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the disease (e.g., cancer); or (2) relieving the disease, e.g., causing regression of symptoms; and (3) preventing or reducing the likelihood of the development of the disease, or preventing secondary diseases/disorders associated with the disease, such as cancer (e.g., cancer metastasis).

An effective amount for the treatment of a disease means that amount which, when administered to a mammal in need thereof, is sufficient to result in effective treatment as that term is defined herein, for that disease. Efficacy of an agent can be determined by assessing physical indicators that are particular to a given disease. For example, physical indicators for cancer include, but are not limited to, pain, tumor size, tumor growth rate, blood cell count, etc.

Target Cells

The DNA construct according to the present invention provide a means for delivering nucleic acids into a broad range of cells, including dividing and non-dividing cells. In one embodiment, the cells are genetically deficient. In one embodiment, the cells are diseased.

The DNA construct can be employed to deliver a nucleic acid of interest to a cell in vitro, e.g., to produce a polypeptide in vitro or for ex vivo gene therapy. The DNA construct are additionally useful in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The DNA construct can also be used to produce a polypeptide of interest or functional RNA in cultured cells or in a subject (e.g., using the subject as a bioreactor to produce the polypeptide or to observe the effects of the functional RNA on the subject, for example, in connection with screening methods).

The cell(s) into which the DNA vector construct is introduced can be of any type, including but not limited to neural cells (including cells of the peripheral and central, nervous systems, in particular, brain, cells such as neurons and oligodendrocytes), lung cells, cells of the eye (including retinal cells, retinal pigment epithelium, and corneal cells), blood vessel cells (e.g., endothelial cells, intimal cells), epithelial cells (e.g., gut and respiratory epithelial cells), muscle, cells (e.g., skeletal muscle cells, cardiac muscle cells, smooth muscle cells and/or diaphragm muscle cells), dendritic cells, pancreatic cells, (including islet cells), hepatic cells, kidney cells, myocardial cells, bone cells (e.g., bone marrow stem cells), hematopoietic stem cells, spleen cells, keratinocytes, fibroblasts, endothelial cells, prostate cells, germ cells, and the, like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin.

Diseases and Disorders

Gene transfer has substantial potential use for understanding and providing therapy for disease states. There are a number of inherited diseases in which defective genes are known and have been cloned. In general, the disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, DNA constructs according to the present invention permit the treatment and/or prevention of genetic diseases.

The DNA vector/construct according to the present invention may also be employed to provide a functional RNA to a cell in vitro or in vivo. Expression of the functional RNA in the cell, for example, can diminish expression of a particular target protein by the cell. Accordingly, functional RNA can be administered to decrease expression of a particular protein in a subject in need thereof. Functional RNA can also be administered to cells in vitro to regulate gene expression and/or cell physiology, e.g., to optimize cell or tissue culture systems or in screening methods. In certain embodiments, the therapeutic targets a protein for correction of a dysregulated cellular pathway of a disease state.

In general, the DNA vector/construct of the present invention can be employed to deliver a nucleic acid encoding a polypeptide or functional RNA to treat and/or prevent any disease state for which it is beneficial to deliver a therapeutic polypeptide or functional RNA. Illustrative disease states include, but are not limited to: cystic fibrosis (cystic fibrosis transmembrane regulator protein) and other diseases of the lung, hemophilia A (Factor VIII), hemophilia B (Factor IX), thalassemia (beta-globin), anemia (erythropoietin) and other blood disorders, Alzheimer's disease (GDF; neprilysin), multiple sclerosis (.beta.-interferon), Parkinson's disease (glial-cell line derived neurotrophic factor (GDNF)), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product), diabetes mellitus (insulin), muscular dystrophies including Duchenne (dystrophin, mini-dystrophin, insulin-like growth factor I, a sarcoglycan (e.g., alpha, beta, gamma), RNAi against myostatin, myostatin propeptide, follistatin, activin type II soluble receptor, anti-inflammatory polypeptides such as the Ikappa B dominant mutant, sarcospan, utrophin, mini-utrophin, RNAi against splice junctions in the dystrophin gene to induce exon skipping (see, e.g., WO/2003/095647), antisense against U7 snRNAs to induce exon skipping (see, e.g., WO/2006/021724), and antibodies or antibody fragments against myostatin or myostatin propeptide) and Becker, Gaucher disease (glucocerebrosidase), Hurler's disease (.alpha.-L-iduronidase), adenosine deaminase deficiency (adenosine deaminase), glycogen storage diseases (e.g., Fabry disease (.alpha.-galactosidase) and Pompe disease (lysosomal acid .alpha.-glucosidase)) and other metabolic defects, congenital emphysema (.alpha.1-antitrypsin), Lesch-Nyhan Syndrome (hypoxanthine guanine phosphoribosyl transferase), Niemann-Pick disease (sphingomyelinase), Tays Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration), diseases of solid organs such as brain (including Parkinson's Disease (GDNF), astrocytomas (endostatin, angiostatin and/or RNAi against VEGF), glioblastomas (endostatin, angiostatin and/or RNAi against VEGF), liver, kidney, heart including congestive heart failure or peripheral artery disease (PAD) (e.g., by delivering protein phosphatase inhibitor I (I-1), serca2a, zinc finger proteins that regulate the phospholamban gene, Barka, beta2-adrenergic receptor, beta2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as TRAP and TNF.alpha. soluble receptor), hepatitis (alpha-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like. The invention can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

In one embodiment, the heterologous nucleic acid further encode reporter polypeptides (e.g., an enzyme). Reporter polypeptides are known in the art and include, but are not limited to, Green Fluorescent Protein, β-galactosidase, alkaline phosphatase, luciferase, and chloramphenicol acetyltransferase gene.

In one embodiment, the heterologous nucleic acid encodes a secreted polypeptide (e.g., a polypeptide that is a secreted polypeptide in its native state or that has been engineered to be secreted, for example, by operable association with a secretory signal sequence as is known in the art).

In one embodiment, the heterologous nucleic acid is operatively linked to a control element, such as transcription/translation control signals, origins of replication, polyadenylation signals, internal ribosome entry sites (IRES), promoters, and/or enhancers, and the like.

Alternatively, in particular embodiments of this invention, the heterologous nucleic acid may encode an antisense nucleic acid, a ribozyme (e.g., as described in U.S. Pat. No. 5,877,022), RNAs that effect spliceosome-mediated trans-splicing (see, Puttaraju et al. (1999) Nature Biotech. 17:246; U.S. Pat. Nos. 6,013,487; 6,083,702), interfering RNAs (RNAi) including siRNA, shRNA or miRNA that mediate gene silencing (see, Sharp et al. (2000) Science 287:2431), and other non-translated RNAs, such as “guide” RNAs (Gorman et al. (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see e.g., Andino et al. J. Gene Med. 10:132-142 (2008) and Li et al. Acta Pharmacol Sin. 26:51-55 (2005)); phospholamban inhibitory or dominant-negative molecules such as phospholamban S16E (e.g., to treat cardiovascular disease, see e.g., Hoshijima et al. Nat. Med. 8:864-871 (2002)), RNAi to adenosine kinase (e.g., for epilepsy), and RNAi directed against pathogenic organisms and viruses (e.g., hepatitis B and/or C virus, human immunodeficiency virus, CMV, herpes simplex virus, human papilloma virus, etc.).

In one embodiment, the DNA vector expresses an immunogenic polypeptide, e.g., for vaccination. An immunogenic polypeptide can be any polypeptide suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env gene products). The immunogenic polypeptide can also be an arenavirus immunogen (e.g., Lassa fever virus immunogen, such as the Lassa fever virus nucleocapsid protein and/or the Lassa fever envelope glycoprotein), a poxvirus immunogen (e.g., a vaccinia virus immunogen, such as the vaccinia L1 or L8 gene product), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpesvirus immunogen (e.g., CMV, EBV, HSV immunogens) a mumps virus immunogen, a measles virus immunogen, a rubella virus immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell. Exemplary cancer and tumor cell antigens are described in S. A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, PRAME, p15, melanoma tumor antigens (Kawakami et al. (1994) Proc. Natl. Acad. Sci. USA 91:3515; Kawakami et al. (1994) J. Exp. Med., 180:347; Kawakami et al. (1994) Cancer Res. 54:3124), MART-1, gp100 MAGE-1, MAGE-2, MAGE-3, CEA, TRP-1, TRP-2, P-15, tyrosinase (Brichard et al. (1993) J. Exp. Med. 178:489); HER-2/neu gene product (U.S. Pat. No. 4,968,603), CA125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (PCT Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, (1996) Ann. Rev. Med. 47:481-91).

The DNA vectors of the present invention can be employed to deliver a heterologous nucleic acid encoding a polypeptide or functional RNA to treat and/or prevent any disease state for which it is beneficial to deliver a therapeutic polypeptide or functional RNA. Illustrative disease states include, but are not limited to: cystic fibrosis (cystic fibrosis transmembrane regulator protein) and other diseases of the lung, hemophilia A (Factor VIII), hemophilia B (Factor IX), thalassemia (3-globin), anemia (erythropoietin) and other blood disorders, Alzheimer's disease (GDF; neprilysin), multiple sclerosis ((3-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the Ikappa B dominant mutant, sarcospan, utrophin, mini-utrophin, antisense or RNAi against splice junctions in the dystrophin gene to induce exon skipping [see e.g., PCT Publication No. WO/2003/095647], antisense against U7 snRNAs to induce exon skipping [see e.g., PCT Publication No. WO/2006/021724], and antibodies or antibody fragments against myostatin or myostatin propeptide) and Becker,

Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic disorders, congenital emphysema (al-antitrypsin), Lesch-Nyhan Syndrome (hypoxanthine guanine phosphoribosyl transferase), Niemann-Pick disease (sphingomyelinase), Tays Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1) and fragments thereof (e.g., I1C), serca2a, zinc finger proteins that regulate the phospholamban gene, Barkct, β2-adrenergic receptor, β2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as TRAP and TNFα soluble receptor), hepatitis (α-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like. The invention can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

The invention can also be practiced to treat and/or prevent a metabolic disorder such as diabetes (e.g., insulin), hemophilia (e.g., Factor IX or Factor VIII), a lysosomal storage disorder such as a mucopolysaccharidosis disorder (e.g., Sly syndrome (β-glucuronidase), Hurler Syndrome (α-L-iduronidase), Scheie Syndrome (α-L-iduronidase), Hurler-Scheie Syndrome (α-L-iduronidase), Hunter's Syndrome (iduronate sulfatase), Sanfilippo Syndrome A (heparan sulfamidase), B (N-acetylgl ucosaminidase), C (acetyl-CoA:α-glucosaminide acetyltransferase), D (N-acetylglucosamine 6-sulfatase), Morquio Syndrome A (galactose-6-sulfate sulfatase), B (β-galactosidase), Maroteaux-Lamy Syndrome (N-acetylgalactosamine-4-sulfatase), etc.), Fabry disease (α-galactosidase), Gaucher's disease (glucocerebrosidase), or a glycogen storage disorder (e.g., Pompe disease; lysosomal acid α-glucosidase).

The invention can also be practiced to treat and/or prevent a lysosomal storage disorder, such as mucopolysaccharidosis disorder (e.g., Sly syndrome (β-glucuronidase), Hurler Syndrome (α-L-iduronidase), Scheie Syndrome (α-L-iduronidase), Hurler-Scheie Syndrome (α-L-iduronidase), Hunter's Syndrome (iduronate sulfatase), Sanfilippo Syndrome A (heparan sulfamidase), B (N-acetylglucosaminidase), C (acetyl-CoA:α-glucosaminide acetyltransferase), D (N-acetylglucosamine 6-sulfatase(, Morquio Syndrome A (galactose-6-sulfate sulfatase), B (β-galactosidase), Maroteaux-Lamy Syndrome (N-acetylgalactosamine-4-sulfatase), etc.), Fabry disease (α-galactosidase), Gaucher's disease (glucocerebrosidase), or a glycogen storage disorder (e.g., Pompe disease; lysosomal acid α-glucosidase) as described herein.

Further, the invention can also be used to produce induced pluripotent stem cells (iPS). For example, a DNA vector of the invention can be used to deliver stem cell associated nucleic acid(s) into a non-pluripotent cell, such as adult fibroblasts, skin cells, liver cells, renal cells, adipose cells, cardiac cells, neural cells, epithelial cells, endothelial cells, and the like. Nucleic acids encoding factors associated with stem cells are known in the art. Nonlimiting examples of such factors associated with stem cells and pluripotency include Oct-3/4, the SOX family (e.g., SOX1, SOX2, SOX3 and/or SOX15), the Klf family (e.g., Klf1, Klf2, Klf4 and/or Klf5), the Myc family (e.g., C-myc, L-myc and/or N-myc), NANOG and/or LIN28.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.”

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The invention is further illustrated by the following examples, which should not be construed as further limiting.

Various embodiments are the invention described herein can further be described in the following two sets of numbered paragraphs.
1. A method for introducing a nucleic acid construct into a target cell for sustained expression comprising administering to the target cell a covalently closed non-viral DNA construct comprising:
   a. at least one DD-ITR comprising:
   i. an inverted terminal repeat having an A, A′, B, B′, C, C′ and D region;
   ii. a D′ region;
   iii. wherein the D and D′ region are complementary palindromic sequences, and wherein D and D′ are positioned adjacent the A and A′ region;
   b. complementary strands of the nucleic acid construct comprising a predetermined DNA sequence that can anneal into expressible dsDNA;
   c. wherein the DNA construct forms linear DNA with covalently closed hairpin ends; and
   d. wherein the DNA construct can express the predetermined DNA sequence in the target cell.
2. The method of paragraph 1, wherein the D regions contain a nicking site.
3. The method of paragraph 1, wherein the D regions are at least 5 nucleotides in length.
4. The method of paragraph 1, wherein the D regions are about 20 nt in length.
5. The method of paragraph 1, wherein the D region corresponds to a parvovirus D region of a parvovirus ITR.
6. The method of paragraph 1, wherein the parvovirus is a dependovirus.
7. The method of paragraph 1, wherein the dependovirus is AAV.
8. The method of paragraph 1, wherein the predetermined DNA sequence is operably linked to a promoter.
9. The method of paragraph 8, wherein the ITR is acting as a promoter.
10. The method of paragraph 8, wherein the promoter is separate from the ITR.
11. The method of paragraph 1-10, wherein the DD-ITR drives expression of the predetermined DNA sequence.
12. The method of paragraph 4, wherein the D and D′ region has a substitution, insertion, and/or deletion that retains at least 5 nucleic acids of the region.
13. The method of paragraph 12, wherein the retained nucleic acids comprise the nicking site and/or junction of the A and A′ region and the D and D′ regions.
14. The method of paragraph 1, wherein the predetermined DNA sequence encodes a protein, a protein fragment, a peptide, or a functional RNA.
15. The method of paragraph 14, wherein the functional RNA is selected from the group consisting of micro RNA, RNAi, shRNA, and guide RNA for Crisper Cas 9 recombination.
16. The method of any of paragraphs 1-15, wherein there are at least 2 nucleotides between the D and D′ region and the predetermined DNA sequence as spacers.
17. The method of paragraph 14, wherein there are at least 2 nucleotides between the D and D′ region and the promoter as spacers.
18. The method of paragraph 16 or 17, wherein the spacers are at least 5 nucleotides.
19. The method of paragraph 16 or 17, wherein the spacers are at least 20 nucleotides.
20. The method of paragraph 16 or 17, wherein the spacers are at least 25 nucleotides.
21. The method of any one of paragraphs 1-20, wherein the at least one DD-ITR is generated from an AAV ITR, a parvovirus ITR, or a synthetic ITR.
22. The method of any one of paragraphs 1-21, wherein the DNA construct comprises two DD-ITRs.
23. The method of any one of paragraphs 1-22, wherein the D regions are from different stereotypes than the ITR.
24. The method of paragraph 22, wherein each DD-ITR is derived from a different viral serotype.
25. The method of paragraph 22 wherein one DD-ITR is derived from an AAV2 ITR, and a second DD-ITR is derived from an AAV5 ITR.
26. The method of any one of paragraphs 1-25, wherein there is a deletion, substitution and/or insertion in the B and B′ or C and C′ region.
27. The method of any one of paragraphs 1-26, wherein there is a deletion, substitution and/or insertion in the A and A′ region.
28. The method of any one of paragraphs 1-27, wherein the DNA construct further comprises a partial protelomerase binding site at the covalently closed ends formed by protelomerase enzyme activity in a host cell.
29. The method of paragraph 28, wherein the host cell expresses the protelomerase under the control of an inducible promoter.
30. The method of any one of paragraphs 1-27, wherein the DNA construct further comprises a partial protelomerase binding site at the covalently closed ends formed by protelomerase enzyme activity in vitro.
31. The method of any one of paragraphs 1-30, wherein the DNA construct persists within the target cell and results in sustained expression of the predetermined sequence.
32. The method of any one of paragraphs 1-31, wherein the DNA construct can be converted into a concatemeric structure in the cell.
33. The method of any one of paragraphs 1-32, wherein the sustained expression of the predetermined DNA sequence in the target cell is for a period of time at least at least 2-5 weeks, at least 1-12 months, at least 1-10 years.
34. The method of any one of paragraphs 32-33, wherein the concatemeric structure persists in the target cell and results in sustained expression of the predetermined sequence.
35. The method of any one of paragraphs 32-34, wherein the concatemeric structure persists in the target cell extra-chromosomally.
36. The method of any one of paragraphs 32-34, wherein the concatemeric structure integrates into the target cell chromosome.
37. The method of any one of paragraphs 1-36 wherein nucleic acid is a therapeutic nucleic acid.
38. The method of any one of paragraphs 1-37, wherein the target cell is in vitro.
39. The method of any one of paragraphs 1-37 wherein the target cell is in vivo.
40. The method of any one of paragraphs 1-37, wherein the construct is administered to the target cell ex vivo.
41. The method of any one of paragraphs 1-40, wherein the target cell is a genetically deficient cell and/or a diseased cell.
42. The method of any one of paragraphs 1-41, wherein the target cell is a diseased cell.
43. The method of any one of paragraphs 1-42, wherein the target cell is selected from the group consisting of a neural cell, lung cell, retinal cell, epithelial cell, smooth muscle cell, skeletal muscle cell cardiac muscle cell, pancreatic cell, hepatic cell, kidney cell, myocardial cell, bone cell, spleen cell, keratinocyte, fibroblast, endothelial cell, prostate cell, germ cell, progenitor cell, stem cell, cancer cell, and tumor cell.
44. A DNA vector for delivery of a predetermined nucleic acid sequence into a target cell for sustained expression, comprising,
   a. two DD-ITRs each comprising:
   i. an inverted terminal repeat having an A, A′, B, B′, C, C′ and D region;
   ii. a D′ region; and
   iii. wherein the D and D′ region are complementary palindromic sequences of about 5-20 nt in length, are positioned adjacent the A and A′ region;
   b. the predetermined nucleic acid sequence (e.g. a heterologous gene for expression); and
      wherein the two DD-ITRs flank the nucleic acid in the context of covalently closed non-viral DNA.
45. The DNA vector of paragraph 44, wherein the predetermined nucleic acid sequence is operably linked to a promoter.
46. The DNA vector of paragraph 44, wherein the DD-ITR drives expression of the predetermined nucleic acid sequence.
47. The DNA vector of paragraph 46, wherein the D and D′ region has a substitution, insertion, and/or deletion that retains at least 5 nucleic acids of the region.
48. The DNA vector of paragraph 47, wherein the retained nucleic acids comprise the nicking site and/or junction of the A and A′ region and the D and D′ regions.
49. The DNA vector of paragraph 44, wherein the predetermined nucleic acid sequence encodes a protein, a protein fragment, a peptide, or a functional RNA.
50. The DNA vector of paragraph 49, wherein the functional RNA is selected from the group consisting of micro RNA, RNAi, shRNA, and guide RNA for Crisper Cas 9 recombination.
51. The DNA vector of paragraph 45, wherein there are at least 2 nucleotides between the D and D′ region and the predetermined nucleic acid sequence as spacers.
52. The DNA vector of paragraph 46, wherein there are at least 2 nucleotides between the D and D′ region and the promoter as spacers.
53. The DNA vector of paragraph 51 or 52 wherein the spacers are at least 5 nucleotides.
54. The DNA vector of paragraph 53, wherein the spacers are at least 20 nucleotides.
55. The DNA vector of paragraph 53, wherein the spacers are at least 25 nucleotides.
56. The DNA vector of any one of paragraphs 44-55, wherein the DD-ITRs are generated from an ITR selected from the group consisting of a parvovirus ITR, and a synthetic ITR.
57. The DNA vector of paragraph 56, wherein the parvovirus is a dependovirus.
58. The DNA vector of paragraph 57, wherein the dependovirus is AAV.
59. The DNA vector of any one of paragraphs 44-58, wherein the DNA construct comprises more than two DD-ITRs.
60. The DNA vector of paragraph 59, wherein each DD-ITR is derived from a different viral serotype.
61. The DNA vector of paragraph 60, wherein one DD-ITR is derived from an AAV2 ITR, and a second DD-ITR is derived from an AAV5 ITR.
62. The DNA vector of any one of paragraphs 44-61, wherein there is a deletion, substitution or insertion in the B and B′ or C and C′ region.
63. The DNA vector of any one of paragraphs 44-61, wherein there is a deletion, substitution or insertion in the A and A′ region.
64. The DNA vector of any one of paragraphs 44-63, wherein the DNA vector further comprises a partial protelomerase binding site and wherein the covalently closed ends are formed by protelomerase enzyme activity in vitro.
65. The DNA vector of any one of paragraphs 44-64, wherein the DNA vector persists within the target cell and results in sustained expression of the predetermined sequence.
66. The DNA vector of any one of paragraphs 44-65, wherein the DNA vector can be converted into a concatemeric structure in the cell.
67. The DNA vector of any one of paragraphs 44-66, wherein the sustained expression of the predetermined DNA sequence in the target cell is for a period of time at least 2-5 weeks, at least 1-12 months, at least 1-10 years.
68. The DNA vector of any one of paragraphs 66-67, wherein the concatemeric structure persists within the target cell and results in sustained expression of the predetermined sequence.
69. The DNA vector of any one of paragraphs 66-68, wherein the concatemeric structure persists in the target cell extra-chromosomally.
70. The DNA vector of any one of paragraphs 66-68, wherein the concatemeric structure integrates into the target cell chromosome.
71. The DNA vector of any one of paragraphs 44-70, wherein predetermined nucleic acid is a therapeutic nucleic acid.
72. The DNA vector of paragraph 44-71 wherein at least one DD-ITR is an AAV ITR.
73. The DNA vector of any one of paragraphs 44-72, wherein the DNA vector further comprises a partial protelomerase binding site flanking the two DD-ITRs.
74. The DNA vector of paragraph 73, wherein the partial protelomerase binding sites flanking the two DD-ITRs are formed by protelomerase enzyme activity in vitro or by protelomerase enzyme activity in vivo.
75. The DNA vector of any one of paragraphs 44-74, wherein the covalently closed non-viral DNA construct persists as a concatemeric structures within the target cell.
76. The DNA vector of paragraph 75, wherein the DNA vector promotes sustained expression of the nucleic acid for a period of time from 2-5 weeks, from 1-12 months, from 1-10 years, or longer.
77. A method for introducing a nucleic acid into a target cell for sustained expression comprising administering to the target cell a covalently closed non-viral DNA construct comprising:
   a. at least one ITR sequence selected from the group consisting of the ITR's shown in FIG. 5;
   b. complementary strands of the nucleic acid construct, wherein the nucleic acid construct comprises a predetermined DNA sequence, wherein the complementary strands can anneal into expressible dsDNA; and
   c. wherein the DNA construct forms linear DNA with hairpin covalently closed ends.
78. The method of paragraph 77, wherein the ITR sequence is flanked on either side by complementary sequences D and D′ to thereby create a DD-ITR.
79. The method of paragraph 77, wherein the predetermined DNA sequence is operably linked to a promoter.
80. The method of paragraph 77, wherein the DD-ITR drives expression of the predetermined DNA sequence.
81. The method of paragraph 77, wherein the D and D′ region has a substitution, insertion, and/or deletion that retains at least 5 nucleic acids of the region.
82. The method of paragraph 81, wherein the retained nucleic acids comprise the nicking site and/or junction of the A and A′ region and the D and D′ regions.
83. The method of paragraph 77, wherein the predetermined DNA sequence encodes a protein, a protein fragment, a peptide, or a functional RNA.
84. The method of paragraph 83, wherein the functional RNA is selected from the group consisting of micro RNA, RNAi, shRNA, and guide RNA for Crisper Cas 9 recombination.
85. The method of paragraph 77-84 wherein there are at least 2 nucleotides between the D and D′ region and the predetermined DNA sequence as spacers.
86. The method of paragraph 77-85, wherein there are at least 2 nucleotides between the D and D′ region and the promoter as spacers.
87. The method of paragraph 85 or 86, wherein the spacers are at least 5 nucleotides.
88. The method of paragraph 87, wherein the spacers are at least 20 nucleotides.
89. The method of paragraph 87, wherein the spacers are at least 25 nucleotides.
90. The method of any one of paragraphs 77-89, wherein the at least one DD-ITR is generated from a parvovirus ITR or a synthetic ITR.
91. The method of paragraph 90 wherein the parvovirus is a dependovirus.
92. The method of paragraph 91, wherein the dependovirus is AAV.
93. The method of any one of paragraphs 65-78, wherein the DNA construct comprises two DD-ITRs.
94. The method of paragraph 93, wherein each DD-ITR is derived from a different viral serotype.
95. The method of paragraph 93, wherein one DD-ITR is derived from an AAV2 ITR, and a second DD-ITR is derived from an AAV5 ITR.
96. The method of any one of paragraphs 77-95, wherein there is a deletion, substitution or insertion in the B and B′ or C and C′ region.
97. The method of any one of paragraphs 77-95, wherein there is a deletion, substitution or insertion in the A and A′ region.
98. The method of any one of paragraphs 77-97, wherein the DNA construct further comprises a partial protelomerase binding site and wherein the covalently closed ends are formed by protelomerase enzyme activity in vitro.
99. The method of any one of paragraphs 77-98, wherein the DNA construct persists within the target cell and results in sustained expression of the predetermined sequence.
100. The method of any one of paragraphs 77-99, wherein the DNA construct can be converted into a concatemeric structure in the cell.
101. The method of any one of paragraphs 77-100, wherein the sustained expression of the predetermined DNA sequence in the target cell is for a period of time at least 1-5 weeks, at least 2-5 weeks, at least 1-12 months, at least 1-10 years.
102. The method of any one of paragraphs 100-101, wherein the concatemeric structure persists within the target cell and results in sustained expression of the predetermined sequence.
103. The method of any one of paragraphs 100-102, wherein the concatemeric structure persists in the target cell extra-chromosomally.
104. The method of any one of paragraphs 100-102, wherein the concatemeric structure integrates into the target cell chromosome.
105. The method of any one of paragraphs 77-104, wherein nucleic acid is a therapeutic nucleic acid.
106. The method of any one of paragraphs 77-105, wherein the target cell is in vitro.
107. The method of any one of paragraphs 77-105, wherein the target cell is in vivo.
108. The method of any one of paragraphs 77-105, wherein the construct is administered to the target cell ex vivo.
109. The method of any one of paragraphs 77-108, wherein the target cell is a genetically deficient cell.
110. The method of any one of paragraphs 77-108, wherein the target cell is a diseased cell.
111. The method of any one of paragraphs 77-110, wherein the target cell is selected from the group consisting of a neural cell, lung cell, retinal cell, epithelial cell, smooth muscle cell, skeletal muscle cell cardiac muscle cell, pancreatic cell, hepatic cell, kidney cell, myocardial cell, bone cell, spleen cell, keratinocyte, fibroblast, endothelial cell, prostate cell, germ cell, progenitor cell, stem cell, cancer cell, and tumor cell.
112. A cell or population thereof, produced by the method of any one of paragraphs 1-43, or 77-111.
113. A covalently closed non-viral linear DNA vector for delivery of predetermined nucleic acid into a target cell for sustained expression comprising
   a. at least one DD-ITR comprising:
      i. an inverted terminal repeat having an A, A′, B, B′, C, C′ and D region;
      ii. a D′ region;
      iii. wherein the D and D′ region are complementary palindromic sequences, and wherein D and D′ are positioned adjacent the A and A′ region;
   b. complementary strands of the nucleic acid construct comprising a predetermined DNA sequence that can anneal into expressible dsDNA;
   c. wherein the DNA vector construct forms linear DNA with covalently closed hairpin ends; and
   d. wherein the DNA vector construct can express the predetermined DNA sequence in the target cell.
114. The DNA vector of paragraph 113, wherein the D regions contain a nicking site.
115. The DNA vector of paragraph 113, wherein the D regions are at least 5 nucleotides in length.
116. The DNA vector of paragraph 113, wherein the D regions are about 20 nt in length.
117. The DNA vector of paragraph 113, wherein the D region corresponds to a parvovirus D region of a parvovirus ITR.
118. The DNA vector of paragraph 113, wherein the parvovirus is a dependovirus.
119. The DNA vector of paragraph 113, wherein the dependovirus is AAV.
120. The DNA vector of paragraph 113, wherein the predetermined DNA sequence is operably linked to a promoter.
121. The DNA vector of paragraph 120, wherein the ITR is acting as a promoter.
122. The DNA vector of paragraph 120, wherein the promoter is separate from the ITR
123. The DNA vector of paragraph 113-122, wherein the DD-ITR drives expression of the predetermined DNA sequence.
124. The DNA vector of paragraph 113, wherein the D and D′ region has a substitution, insertion, and/or deletion that retains at least 5 nucleic acids of the region.
125. The DNA vector of paragraph 124, wherein the retained nucleic acids comprise the nicking site and/or junction of the A and A′ region and the D and D′ regions.
126. The DNA vector of paragraph 113, wherein the predetermined DNA sequence encodes a protein, a protein fragment, a peptide, or a functional RNA.
127. The DNA vector of paragraph 126, wherein the functional RNA is selected from the group consisting of micro RNA, RNAi, shRNA, and guide RNA for Crisper Cas 9 recombination.
128. The DNA vector of any of paragraphs 1-127, wherein there are at least 2 nucleotides between the D and D′ region and the predetermined DNA sequence as spacers.
129. The DNA vector of paragraph 126, wherein there are at least 2 nucleotides between the D and D′ region and the promoter as spacers.
130. The DNA vector of paragraph 128 or 129, wherein the spacers are at least 5 nucleotides.
131. The DNA vector of paragraph 128 or 129, wherein the spacers are at least 20 nucleotides.
132. The DNA vector of paragraph 128 or 129, wherein the spacers are at least 25 nucleotides.
133. The DNA vector of any one of paragraphs 113-132, wherein the at least one DD-ITR is generated from an AAV ITR, a parvovirus ITR, or a synthetic ITR.
134. The DNA vector of any one of paragraphs 113-133, wherein the DNA vector construct comprises two DD-ITRs.
135. The DNA vector of any one of paragraphs 113-134, wherein the D regions are from different stereotypes than the ITR.
136. The DNA vector of paragraph 134, wherein each DD-ITR is derived from a different viral serotype.
137. The DNA vector of paragraph 134, wherein one DD-ITR is derived from an AAV2 ITR, and a second DD-ITR is derived from an AAV5 ITR.
138. The DNA vector of any one of paragraphs 113-137, wherein there is a deletion, substitution and/or insertion in the B and B′ or C and C′ region.
139. The DNA vector of any one of paragraphs 113-138, wherein there is a deletion, substitution and/or insertion in the A and A′ region.
140. The DNA vector of any one of paragraphs 113-139, wherein the DNA vector construct further comprises a partial protelomerase binding site and wherein the covalently closed ends are formed by protelomerase enzyme activity in vitro.
141. The DNA vector of any one of paragraphs 113-140, wherein the DNA vector construct persists within the target cell and results in sustained expression of the predetermined sequence.
142. The DNA vector of any one of paragraphs 113-141, wherein the DNA vector construct can be converted into a concatemeric structure in the cell.
143. The DNA vector of any one of paragraphs 113-142, wherein the sustained expression of the predetermined DNA sequence in the target cell is for a period of time at least at least 2-5 weeks, at least 1-12 months, at least 1-10 years.
144. The DNA vector of any one of paragraphs 142-143, wherein the concatemeric structure persists in the target cell and results in sustained expression of the predetermined sequence.
145. The DNA vector of any one of paragraphs 142-143, wherein the concatemeric structure persists in the target cell extra-chromosomally.
146. The DNA vector of any one of paragraphs 142-143, wherein the concatemeric structure integrates into the target cell chromosome.
147. The DNA vector of any one of paragraphs 113-146 wherein nucleic acid is a therapeutic nucleic acid.
148. A pharmaceutical composition for delivery of a nucleic acid to a target cell comprising the DNA vector of any of paragraphs 44-76 and 113-147 and pharmaceutically acceptable carrier for delivery into a target cell, wherein the target cell is selected from the group consisting of a neural cell, lung cell, retinal cell, epithelial cell, smooth muscle cell, skeletal muscle cell cardiac muscle cell, pancreatic cell, hepatic cell, kidney cell, myocardial cell, bone cell, spleen cell, keratinocyte, fibroblast, endothelial cell, prostate cell, germ cell, progenitor cell, stem cell, cancer cell, and tumor cell.
149. The pharmaceutical composition of paragraph 148, wherein the composition is administered to the target cell in vivo for treatment of a disease or disorder.
150. The method of paragraph 28, wherein the host cell has been designed to encode at least a first Tel recombinase under the control of an inducible promoter, wherein said cell comprises an expression vector adapted to produce a bacterial sequence-free vector, said vector comprising an expression cassette, and a nucleic acid of interest are flanked by at least one DD-ITR, and on either side by a target sequence for the Tel recombinase.
151. The method of paragraph 150, wherein integrated within non-binding regions of the Tel target sequence are target binding sequences for one or more additional recombinases.
152. The method of paragraph 151, wherein the one or more additional recombinases is selected from the group consisting of pK02 telRL site, the telRL site, the pal site, the loxPsite, the FRT site, phiC31, attP site and the XattP site.
153. A method of producing a linear covalently closed vector containing at least one DD-ITR comprising incubating the host cell of paragraph 150 under conditions suitable to permit expression of the first recombinase to result in a linear covalently closed vector.
154. A method of producing a circular covalently closed vector containing at least one DD-ITR comprising incubating the host cell of paragraph 151-152 under conditions suitable to permit expression of a second recombinase to result in circular covalently closed vector.
155. The method of paragraph 150, wherein the Tel recombinase target site is the phage PY54 Tel 142 base pair target site.
156. The method of paragraphs 150-155 wherein the nucleic acid of interest are flanked on both sides by DD-ITRs.

Second set of numbered paragraphs:
1. A method of manufacturing circular nucleic acid vectors containing a transgene, the method comprising:
   a. contacting a host system with a template, wherein the template comprises at least one flanking cleavage site, and:
      i. at least one phage origin of replication (ORI);
      ii. at least one Terminal Repeat (TR), and;
      iii. a promoter sequence operatively linked to a transgene,
      wherein at least one TR is an adeno associated virus (AAV) Double D ITR (DD-ITR);
   b. incubating the host system for a time sufficient for replication to occur resulting in circular nucleic acid production; and
   c. recovering the circular nucleic acid produced, wherein the circular nucleic acid self-anneals.
2. The method of paragraph 1, wherein the template further comprises a second flanking cleavage sites, and within the two sites are (i)-(iii).
3. The method of paragraphs 1 and 2, wherein the template further comprises at least one additional cleavage site immediately downstream of the at least one ORI.
4. The method of any of paragraphs 1-3, further comprising the step of cutting at least one cleavage site of the recovered circular nucleic acid.
5. The method of any of paragraph 1, further comprising, following recovery, the step of in vitro replication of the circular nucleic acid.
6. The method of paragraph 1, wherein the template further comprises at least one adapter sequence.
7. The method of paragraph 1, wherein the template further comprises at least two adapter sequences.
8. The method of paragraphs 6 or 7, wherein the adaptor sequence induces closure of cleaved DNA.
9. The method of paragraphs 6 or 7, wherein the adaptor sequence further comprises a cleavage site.
10. The method of any of paragraphs 1-9, wherein the recovered circular nucleic acid is used for delivery of the transgene.
11. The method of any of paragraphs 1-9, wherein the recovered circular nucleic acid is used for recombinant viral vector production.
12. The method of paragraph 1, wherein the circular nucleic acid is self-annealed and double-stranded.
13. The method of paragraph 1, wherein the vector is single-stranded.
14. The method of paragraph 1, wherein there is a second TR and the promoter sequence operably linked to a transgene is flanked on both sides by a TR.
15. The method of paragraph 1, wherein the ORI is upstream of the left TR.
16. The method of paragraph 1, wherein the ORI is flanked by the TRs and upstream of the promoter sequence operably linked to a transgene.
17. The method of paragraph 1, wherein the host system is a bacterial packaging cell.
18. The method of paragraph 1, wherein the host system is a cell-free system.
19. The method of paragraph 1, wherein the host system is a cell-free system and contains helper phage particles.
20. The method of paragraph 1, wherein the host system is a host cell.
21. The method of paragraph 20, wherein the host cell is a mammalian cell, a bacterial cell, or an insect cell.
22. The method of paragraph 11, wherein the viral vector is an adeno associated virus (AAV), a lentivirus (LV), a herpes simplex virus (HSV), an adeno virus (AV), or a pox virus (PV).
23. The method of paragraphs 11 and 22, wherein the viral vector is a DNA or RNA virus.
24. The method of paragraph 22, wherein the virus is an AAV and has a mutant ITR, wherein the mutant ITR is a Double D mutant ITR.
25. The method of paragraph 1, wherein the at least one TR is a mutant ITR, a synthetic ITR, a wild-type ITR, or a non-functional ITR.
26. The method of paragraph 1, wherein the vector has flanking DD-ITRs, and in between the flanking DD-ITRs is a promoter operatively linked to a sense strand of the transgene, a replication defective ITR, and an anti-sense complement of the transgene.
27. The method of paragraphs 25-26, wherein the ITR is an AAV ITR
28. The method of paragraph 1, wherein the ORI is located upstream of the ITR, and immediately downstream of the upstream ITR.
29. The method of paragraph 1, wherein the at least one phage ORI is selected from the group consisting of: M13 derived ORI, F1 derived ORI, or Fd derived ORI.
30. The method of paragraph 1, wherein the temple further comprises a second ORI that is a truncated ORI that does not initiate replication.
31. The method of paragraph 30, wherein the truncated ORI is ORIΔ29.
32. The method of paragraph 1, wherein the at least two cleavage sites are a restriction site.
33. The method of paragraph 32, wherein the at least two restriction sites are identical or different.
34. The method of paragraph 32, wherein the restriction site is not found within the transgene sequence.
35. The method of paragraph 1, wherein the cleavage site is cleaved by a nuclease.
36. The method of paragraph 1, wherein the promotor is selected from the group consisting of: a constitutive promoter, a repressible promoter, a ubiquitous promoter, an inducible promoter, a viral promoter, a tissue specific promoter, and a synthetic promoter.
37. The method of paragraph 1, wherein the transgene is a therapeutic gene.
38. A method of manufacturing circular nucleic acid vectors containing a transgene, the method comprising:
   a. transforming a host system with a plasmid template, wherein the plasmid template comprises:
      i. a phage origin of replication (ORI);
      ii. a truncated phage ORI (e.g., ORIΔ29);
      iii. at least one Terminal Repeat (TR), and;
      iv. a promoter sequence operatively linked to a transgene, wherein the plasmid template comprises, in the 5′ to 3′ direction, the sense sequence and the anti-sense sequence separated by a hairpin sequence that allows for annealing of the sense and anti-sense strand, and wherein at least one TR is an AAV Double D ITR (DD-ITR);
   b. incubating the host system for a time sufficient for replication to occur resulting in circular nucleic acid production; and
   c. recovering the circular nucleic acid produced,
   wherein the circular nucleic acid self-anneals.
39. The method of paragraph 38, further comprising a linker and a self-complement linker flanking the ORI.
40. The method of paragraph 38, wherein the transgene contains the sense sequences and the anti-sense complement thereof separated by a linker sequences that will permit the sense and anti-sense strands to bind as a double strand.
41. The method of paragraph 38, wherein the truncated ORI is ORIΔ29.
42. A circular nucleic acid vector manufactured by the methods of any of paragraphs 1-41.
43. A circular nucleic acid vector comprising:
   at least one flanking cleavage site, and:
   i. at least one phage origin of replication (ORI);
   ii. at least one Terminal Repeat (TR); and
   iii. a promoter sequence operatively linked to a transgene,
   wherein at least one TR is an AAV Double D ITR (DD-ITR).
44. The vector of paragraph 43, wherein the template further comprises a second flanking cleavage sites, and within the two sites are (i)-(iii).
45. The vector of paragraphs 43 and 44, wherein the vector further comprises at least one additional cleavage site immediately downstream of the at least one ORI.
46. The vector of paragraph 43, wherein the vector further comprises at least one adapter sequence.
47. The vector of paragraph 43, wherein the vector further comprises at least two adapter sequences.
48. The vector of paragraphs 46 and 47, wherein the adaptor sequence induces closure of cleaved DNA.
49. The vector of paragraphs 46 and 47, wherein the adaptor sequence further comprises a cleavage site.
50. The vector of any of paragraphs 43-49, wherein the vector is used for delivery of the transgene.
51. The vector of any of paragraphs 43-49, wherein the vector is used for recombinant viral vector production.
52. The vector of paragraph 43, wherein the vector is self-annealed and double-stranded.
53. The vector of paragraph 43, wherein the vector is single-stranded.
54. The vector of paragraph 43, wherein there is a second TR and the promoter sequence operably linked to a transgene is flanked on both sides by a TR.
55. The vector of paragraph 43, wherein the ORI is upstream of the left TR.
56. The vector of paragraph 43, wherein the ORI is flanked by the TRs and upstream of the promoter sequence operably linked to a transgene.
57. The vector of paragraph 43, wherein the at least one TR is a mutant ITR, a synthetic ITR, a wild-type ITR, or a non-functional ITR.
58. The vector of paragraph 43, wherein the vector has flanking DD-ITRs, and in between the flanking is a promoter operatively linked to a sense strand of the transgene, a replication defective ITR, and an anti-sense complement of the transgene.
59. The vector of paragraphs 57 and 58, wherein the ITR is an AAV ITR.
60. The vector of paragraph 43, wherein the ORI is located upstream of the ITR, and immediately downstream of the upstream ITR.
61. The vector of paragraph 43, wherein the phage ORI is selected from the group consisting of: M13 derived ORI, F1 derived ORI, or Fd derived ORI.
62. The vector of paragraph 43, wherein the temple further comprises a second ORI that is a truncated ORI that does not initiate replication.
63. The vector of paragraph 43, wherein the truncated ORI is ORIΔ29.
64. The vector of paragraph 43, wherein the at least two cleavage sites are a restriction site.
65. The vector of paragraph 64, wherein the at least two restriction sites are identical or different.
66. The vector of paragraph 64, wherein the restriction site is not found within the transgene sequence.
67. The vector of paragraph 43, wherein the cleavage site is cleaved by a nuclease.
68. The vector of paragraph 43, wherein the promotor is selected from the group consisting of: a constitutive promoter, a repressible promoter, a ubiquitous promoter, an inducible promoter, a viral promoter, a tissue specific promoter, and a synthetic promoter.
69. The vector of paragraph 43, wherein the transgene is a therapeutic gene.
70. A circular nucleic acid vector comprising:
   i. a phage origin of replication (ORI);
   ii. a truncated phage ORI (e.g., ORIΔ29);
   iii. at least one Terminal Repeat (TR), and;
   iv. a promoter sequence operatively linked to a transgene,
   wherein the vector comprises, in the 5′ to 3′ direction, the sense sequence and the anti-sense sequence separated by a hairpin sequence that allows for annealing of the sense and anti-sense strand, and
   wherein at least one TR is an AAV Double D ITR (DD-ITR).
71. The vector of paragraph 70, further comprising a linker and a self-complement linker flanking the ORI.
72. The vector of paragraph 70, wherein the transgene contains the sense sequences and the anti-sense complement thereof separated by a linker sequences that will permit the sense and anti-sense strands to bind as a double strand.
73. The vector of paragraph 70, wherein the truncated ORI is ORIΔ29.
74. The method of paragraph 35, wherein the nuclease is a protelomerase.
75. The vector of paragraph 67, wherein the nuclease is a protelomerase.

### Examples

### Example 1: Production of Covalently Closed Linear Vector

A. Synthesis of Double D ITR Sequences

The Polymerase Chain Reaction (PCR) is used to construct the inverted terminal repeat with a D′ sequence added to the other end. The rationale is based on the T-shape structure of the ITR. In the first round of PCR reaction, the AAV viral IRT will self-prime the elongation to produce a long T-shaped hairpin structure containing D and D′ on the stem. Upon denaturation, this DNA can serve as template for single-primed PCR.

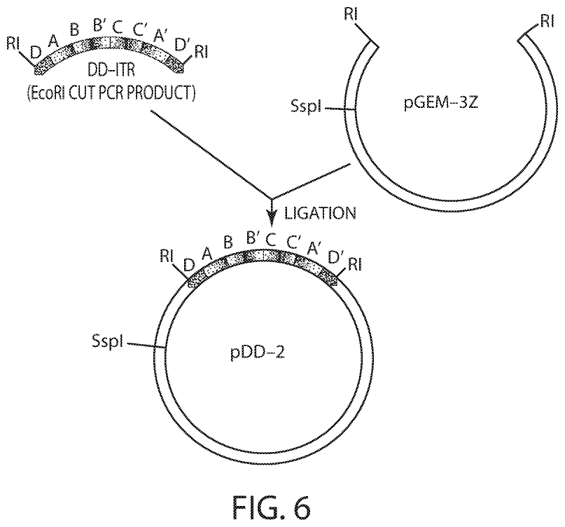

Owing to the high GC content and the strong palindromic structure in the ITR region, several strategies such as 7-deazo-dGTP, 2.5% formamide, and high concentration of primer can be utilized to tackle the PCR problems and yield sufficient desired PCR product. For the convenience of cloning, an EcoRI recognition sequence is attached to the 5′ of the primer so that the PCR product can be cut by EcoRI and easily cloned into the polylinker of pGEM 3Z. Due to the instability of the ITR in bacteria host, the recombinant plasmid is transformed into an E. coli SURE strain (Stratagene) in which the ITR is rather stable. By using the above strategy, numerous positive clones can be obtained. Some clones are characterized by restriction digestion and sequencing. One of the clones shown to bear an insert of D′ABB′CC′A′D in the EcoRI site of the pGEM3Z will be used. This generated plasmid named pDD-2 (FIG. 6) is an exemplary source of a DD-ITR.

Materials and Methods

B. PCR and Construction of ITR Plasmid. Low molecular weight DNA from AAV and Ad5 infected cells is used as template for the PCR reaction with a single primer derived from D-sequence of AAV. The PCR is performed at 94° C. 1 min., 45° C. 30 seconds and 72° C. 1 min. for 35 cycles in a 50 ul reaction solution containing 20 mM Tris-HCl (pH8.8), 1.5 mM MgCl, 50 mM KCl, 2.5% formamide, 100 uM dATP, dCTP and dTTP, 75 uM 7-deazo-dGTP, 25 uM dGTP, 1.5U AmpliTaq (Perkin Elmer Cetus), 1 ng AAV DNA and 100 pmole primer TR-1 (5′-GGAATTCAGGAACCCCTAGTGATGG3-3′). The PCR product is purified by agarose gel electrophoresis, cut with EcoRI and ligated with an EcoRI cut and dephosphorylated pGEM 3Z plasmid (Promega). The ligated plasmid is transformed into E. coli Sure strain (Stratagene). Positive clones are screened for the presence of a double-D terminal repeat and confirmed by dideoxy-sequencing with 7-deazo-dGTP substituted for dGTP (Sanger, F. et al., 1977, Proc. Natl. Acad. Sci. U.S.A. 74:5463-5467). Subsequently, a neogene is cloned into the SalI site of pDD resulting in the plasmid pDD-neo. pDD-neo is used as a source of the DD-ITR

C. Production of Closed Linear DNA from a Double Stranded Circular DNA Template

Double stranded circular DNA containing a luciferase reporter gene in expressible form, the DD-ITR, and a protelomerase TelN binding sequence is used as the DNA template. A single palindromic oligonucleotide complementary to a section of one half of the palindromic sequence that comprises the protelomerase TelN binding site is used to specifically prime both strands. Examples of suitable primers include:

(SEQ ID NO: 16)
CGCATATTACCYWTAACACAC;
(SEQ ID NO: 17)
GCGTATAATGGRCWATTGTGTG;
(SEQ ID NO: 18)
GCGTATAATGG;
(SEQ ID NO: 19)
CCATTATACGC;
(SEQ ID NO: 20)
CACACAATWGYCCAT;
(SEQ ID NO: 21)
ATGGRCWATTGTGTG;

where in Y is T or C, W is A or T, and R is A or G. Denaturation of the double stranded circular template and the annealing of the single primer is carried out in an annealing/denaturation buffer containing, for example, 30 mM Tris-HCl pH 7.5, 20 mM KCl, 2.5 mM MgCl₂. Denaturation is carried out by heating to 95° C. and maintaining at this temperature for 1 to 10 minutes followed by a carefully controlled cooling profile optimised for the maximum binding of the specific primer to the template. The temperature is then reduced to the optimum for DNA amplification by a suitable DNA polymerase. A suitable enzyme is phi29 isolated from the Bacillus subtilis phage phi29 that works optimally at 30° C.

A suitable volume of reaction buffer containing the enzymes phi29 and PPi (Yeast Inorganic pyrophosphatase), is then added to the annealed DNA/primer reaction. The reaction mixture is incubated at around 30° C. for between 5 and 20 hours or longer. A suitable reaction buffer typically contains 35 mM Tris-HCl, 50 mM KCl, 2.5 mM MgCl₂, 10 mM (NH₄)₂SO₄, 4 mM DTT, 1 mM dNTP.

Concatemeric DNA amplified by RCA is then incubated at 30° C. with the protelomerase TelN in a suitable buffer such as 10 mM Tris HCl pH 7.6, 5 mM CaCl₂, 50 mM potassium glutamate, 0.1 mM EDTA, 1 mM DTT until the reaction is complete. The resulting closed linear DNA product may be purified, for example, by gel electrophoresis or a suitable chromatographic method depending on the amount to be purified.

D. Production of Closed Linear DNA from a Closed Linear DNA Template

Closed linear DNA containing a luciferase reporter gene in expressible form, the DD-ITR, and telomeric ends comprising the binding sequence of a protelomerase TelN is used as the DNA template. Constructs that have spacers between the DD-ITR and the promoter of the luciferase reporter gene but are otherwise identical are generated. Constructs with no spacers, and with spacers of 2, 5, 10, 20 and 25 nucleotides are generated. A single palindromic oligonucleotide complementary to a section of one half of the palindromic sequence that comprises the telomeric ends of the template is used as a specific primer. The primer binds to two identical sites on the DNA template. Examples of suitable primers include those shown in the above example.

Denaturation of the closed linear DNA template and the annealing of the single primer is carried out in an annealing/denaturation buffer containing, for example, 30 mM Tris-HCl pH 7.5, 20 mM KCl, 2.5 mM MgCl₂. Denaturation is carried out by heating to 95° C. for 1 min and maintaining at this temperature for 1 to 10 minutes followed by a carefully controlled cooling profile optimised for the maximum binding of the specific primer to the template. The temperature is then reduced to the optimum for DNA amplification by a suitable DNA polymerase. One such suitable enzyme is phi29 isolated from the Bacillus subtilis phage phi29 that works optimally at 30° C.

A suitable volume of reaction buffer containing the enzymes phi29 and PPi (Yeast Inorganic pyrophosphatase) is then added to the annealed DNA/primer reaction. The reaction mixture is incubated at around 30° C. for between 5 and 20 hours or longer. A suitable reaction buffer typically contains 35 mM Tris-HCl, 50 mM KCl, 2.5 mM MgCl₂, 10 mM (NH₄)₂SO₄, 4 mM DTT, 1 mM dNTP.

Concatameric DNA amplified by RCA is then incubated at 30° C. with the protelomerase TelN in a suitable buffer such as 10 mM Tris HCl pH 7.6, 5 mM CaCl₂), 50 mM potassium glutamate, 0.1 mM EDTA, 1 mM DTT until the reaction is complete. The resulting closed linear DNA product may be purified, for example, by gel electrophoresis or a suitable chromatographic method depending on the amount to be purified.

These methods provide for a cyclic reaction wherein the product is identical to the template. This reaction is easily scaled up from a very small amount of template by carrying out additional cycles of the methods steps.

### Example 2: Construction of Covalently Closed Linear Vector by Cell-Lines Containing Inducible Recombinases

Strains and Plasmids

E. coli K-12 strains are used in the generation of all recombinant cell constructs and DH5α and JM109, in particular, are employed as hosts for plasmid constructions and amplification.

Construction of Recombinant Cells (R-Cells)

W3110 (recA⁺) E. coli is used for chromosomal engineering studies and in vivo recombinase-expression as follows. Protelomerase coding gene tel is amplified from bacteriophage PY54 lysate using the following primers: Tel-F 5′-GCGGATCCTGGGTTACTTTAATTTGTGTGTT-3′ (SEQ ID NO: 22) and Tel-R 5′-CGCTCGAGTTACTCCATATTTTCAGTCCATGCTTGT-3′ (SEQ ID NO: 23) (annealing Tm 64° C.). Protelomerase coding gene telN is amplified from bacteriophage N15 lysate using primers: TelN-F 5′-ATCGGATCCCGATATCCAGAGACTTAGAAACGGG-3′ (SEQ ID NO: 24) and TelN-R 5′-ATATAAAGCTTCTTTTAGCTGTAGTACGTTTCCCATGCG-3′ (SEQ ID NO: 25) (annealing Tm 62° C.). As a positive control for in vivo production of modified pDNA vectors, the recombinase encoding gene cre is amplified from bacteriophage Plrev6 lysate using primers: Cre-F 5′-GGAAATTCCGGTCGCTGGCGTTTCTATGAC-3′ (SEQ ID NO: 26) and Cre-R 5′-CGCTCGAGTGAATATTAGTGCTTACAGACAG-3′ (SEQ ID NO: 27) (annealing Tm 66° C.). Italicized regions denote restriction sites for BamHI, XhoI, HindIII, and EcoRI. PCR amplifications is conducted using Phusion Flash High-Fidelity PCR Master Mix (New England Biolab) for 30 s at 98° C. for initial denaturation, 30 cycles of 5 s at 98° C., 10 s at annealing Tm, 45 s at 72° C., and 2 min at 72° C. for final extension to generate cre (1.3 kb), tel (2.1 kb), and telN (2.3 kb) fragments. Constructs are tested and confirmed by colony PCR and analytical digestion. PCR products are purified from 0.8% agarose gel (Qiagen Gel extraction kit), and digested with the listed enzymes (New England Biolabs). Recombinase genes are cloned into the MCS of the inducible prokaryotic expression vector pPL451 (Accession #AB248919) to produce pNN1, pNN2, and pNN3 vectors. pPL451 (4.2 kb) imparts temperature-regulated expression of the cloned gene via CI[Ts]857-mediated repression of the λ P_{L} strong promoter. All primers are designed using the Gene Runner 3.01 (Hastings Software, Inc) and synthesized commercially (Sigma-Aldrich, Inc). R-cells are constructed via insertion of recombinase genes into E. coli W3110 chromosome using the pBRINT-Cm integrating plasmids, which facilitates the homologous recombination and chromosomal integration of cloned sequence of interest into the lacZ gene of E. coli. For each plasmid construct encoding inducible expression of a cloned recombinase in pPL451, the cI857-P_{L}-X-t_{L}, cassette (where X=cre, tel or telN) is amplified from the pNN1 to 3 constructs by the cI857X-F 5′-TCCCCGCGGAGCTATGACCATGATTACGAATTGC-3′ (SEQ ID NO: 28), cI857telN/cre-R 5′-GGACTAGTCCCCATTCAGGCTGCGCAACTGTTG-3′ (SEQ ID NO: 29), and cI857tel-R 5′-GCTCTAGAGCAGGCTGCGCAACTGTTGGGAAG-3′ (SEQ ID NO: 30) primers with SacII, SpeI, and XbaI sites respectively. The amplified cassettes are cloned into the MCS of pBRINT (Cm^{R}) plasmid to produce pNN4, pNN5, and pNN6 integrating vector constructs. Amplification is performed by the Phusion Flash High-Fidelity PCR Master Mix (New England Biolab) for 10 s at 98° C. for initial denaturation, 30 cycles of is at 98° C., 5 s at 68° C., 120 s at 72° C., and 1 min at 72° C. for final extension to generate cI857-cre (2.8 kb), cI857-tel (3.2 kb), and cI857-telN (3.5 kb) fragments. Constructs are tested and confirmed by colony PCR and analytical digestion.

The multi-purpose recombinase target site, named Supersequenee (SS), is designed to carry Cre, Flp and TelN minimal targets sites (loxP-FRT-telRL) respectively, all within the Tel 142 bp pal sequence, which provides the PY54 derived Tel target site. SS also carries a 78 bp SV40 enhancer sequence that flanks each side of the pal sequence to facilitate nuclear translocation and enhancing transfection efficiency. The SS fragment is synthesized by the GeneScript and cloned into the pUC57 by EcoRI and HindIII.

The plasmid described in Examples 1A and B above (5.8 kb) (Promega), is modified by replacement of the luc gene (1.65 kb) with egfp (790 bp) from pGFP (clontech) to form pDDEGFR. Next, SS is cloned immediately upstream of the SV40 promoter+intron site of pDDEFGR to form pDD3. Then the SS fragment is cloned downstream of the poly A site of pDD3 to form pDD4. This plasmid carries 2 SS sites that flank the EGFP gene cassette.

The multicopy plasmid can be conformed to mini circular (ccc) vector (mediated by Cre-loxP; Flp/FRT), or mini linear covalently closed (lee) vector (mediated by TelN-telRL; Tel/pal). R-cells are transformed by 1 ug of pNN7 to 9 DNA constructs on LB+Ap (50 ug/ml) to A₆₀₀=0.6 at 30° C. with aeration. To induce recombinase expression and plasmid conformational conversion, transformed R-cells are heat shocked to induce the recombinase expression at 42° C. for 30 min at mid-logarithmic phase of bacterial growth, before being transferred to 30° C. overnight. Cells are then harvested and plasmid extracted (Omega mini plasmid extraction kit, VWR). Plasmid topology is assayed by agarose gel electrophoresis and digestion. Standard recombinant DNA techniques is performed as described by Sambrook et al. (1989).

R-Cells Exhibit Temperature-Regulated Recombinase Expression

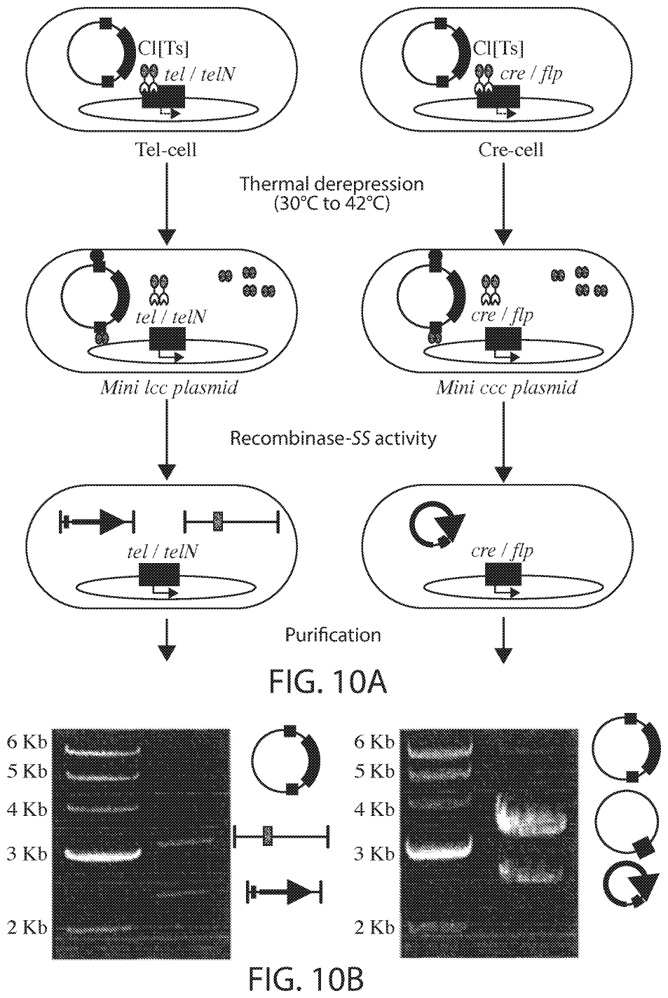

Recombinant cells are constructed that place tel or telN recombinase genes under control of the bacteriophage λ strong promoter, pL, that is regulated by the temperature-sensitive λ repressor, CI[Ts]857 (FIG. 10). Positive cre-expressing control cells are similarly prepared. Total cellular protein in tel and telN R-cells under repressed and fully induced (42° C.) conditions are examined. Both R-cells demonstrate minimal recombinase protein levels, identified at 72 KDa for both TelN and Tel at 30° C., where CI[Ts] actively binds the O_{L} operator and represses transcription of the downstream recombinase gene. Upon shifting cells to 42° C., where repressor activity is completely abrogated and occlusion of pL promoter activity is relieved, prominent recombinase expression is observed. These results confirm that the constructed Tel- and Tel-N cells are temperature inducible for recombinase production.

To determine the fate of cells upon linearizing/disrupting the E. coli chromosome, λ site-specific SS⁻ and SS⁺ integrants are incubated under conditions that provide none to very low (30° C.) or full (42° C.) expression of the recombinase, then cell viability is measured. Under repressed conditions (30° C.), all recombinant cells retain near full viability regardless of the presence or absence of the SS integrated in the chromosome. However, upon shifting temperature to 42° C. and inducing expression of Tel or TelN, recombinants that carried SS⁺ shows dramatically reduced viability. And, in both systems, Tel-cells results in approximately 5-fold greater killing than that seen in TelN-cells.

### Example 3—Linear Covalently Closed DNA Generated by the Prokaryotic Cleaving-Joining Enzyme TelN is Functional in Mammalian Cells

### Materials and Methods

Mice and Cell Lines

C57BL/6 mice are used at 6-8 weeks of age. HEK293 cells are grown in Dulbecco's Modified Eagles Medium supplemented with 10% fetal calf serum, 100 μg/ml streptomycin, and 100 IU/ml penicillin. HEK293 cells are transiently transfected using lipofectamin (Invitrogen, Life Technologies, Carlsbad, USA) or calcium phosphate precipitation with a mixture of the telRL-carrying expression construct with or without pEGFPc2-CNK-CT as an internal control for transfection efficiency.

Construction of Recombinant Plasmids

A plasmid containing a Double D ITR sequence and a reporter gene can be made as described in Examples 1A and 1B or using a Double D ITR plasmid modified by insertion of a SalI/NotI enhanced green fluorescent protein (EGFP) [Hartikka J, Sawdey M, Cornefert-Jensen F, Margalith M, Barnhart K, Nolasco M, Vahlsing H L, Meek J, Marquet M, Hobart P, Norman J, Manthorpe M (1996) An improved plasmid DNA expression vector for direct injection into skeletal muscle. Hum Gene Ther 7:1205-1217, See Example 2]. A telRL site derived from pJD105 [Deneke J, Ziegelin G, Lurz R, Lanka E (2000) The protelomerase of temperate Escherichia coli phage N15 has cleaving-joining activity. Proc Natl Acad Sci USA 97:7721-7726] can be inserted as a 222-bp PvuII/DpnI fragment harboring the 56-bp telRL site into the filled-in NdeI site of pIRES.mu-IL12 [Schultz J, Pavlovic J, Strack B, Nawrath M, Moelling K (1999) Long-lasting anti-metastatic efficiency of interleukin 12-encoding plasmid DNA. Hum Gene Ther 10:407-417] and pVR1012.EGFP upstream of the CMV promoter. A second telRL site is inserted into the filled-in BstXI site of the plasmid, located downstream of the polyA signal.

Preparation of Linear DNA with Covalently Closed Ends

The circular covalently closed coiled plasmid DNA (ccc-DNA) of the plasmid is converted to linear DNA with covalently closed ends (lc-DNA) using purified protelomerase TelN as described [Deneke J, Ziegelin G, Lurz R, Lanka E (2000) The protelomerase of temperate Escherichia coli phage N15 has cleaving-joining activity. Proc Natl Acad Sci USA 97:7721-7726]. lc-DNA is extracted with phenol/chloroform, washed with chloroform, and precipitated twice with ethanol. The same procedure is applied to DNA linearized by restriction enzymes and ccc-DNA. In the case of these plasmids the mini lc- and lo-DNAs are separated from the backbone fragments on 1.2% agarose gels and purified using Elutip columns (Schleicher and Schuell, Dassel, Germany).

Determination of EGFP

The expression of EGFP in transiently transfected HEK293 cells is determined 48 h post transfection by Western blot analysis as described previously [Zimmermann S, Rommel C, Ziogas A, Lovric J, Moelling K, Radziwill G (1997) MEK1 mediates a positive feedback on Raf-1 activity independently of Ras and Src. Oncogene 15:1503-1511] using monoclonal anti-GFP antibody (Clontech) and the ECL detection system (Amersham Pharmacia Biotech, Piscataway, USA).

Results

Linear Closed Plasmid DNAs Derived by the TelN/telRL System.

In order to express therapeutic molecules from DNA, a transcriptional unit consisting of promoter, intron A, gene of interest, and polyadenylation signal is sufficient. When DNA fragments containing a transcriptional unit are generated using restriction endonucleases, the resulting DNA molecules are sensitive for degradation by exonucleases. Most naturally occurring DNA molecules have covalently closed ends for stabilization. Mechanisms to achieve this are circularization, integration into the host genome, or formation of covalently closed hairpin structures. The latter mechanism yields a linear DNA molecule. The prokaryotic telomerase, protelomerase TelN, of bacteriophage N15 is shown to exert this function in vitro [Deneke J, Ziegelin G, Lurz R, Lanka E (2000) The protelomerase of temperate Escherichia coli phage N15 has cleaving-joining activity. Proc Natl Acad Sci USA 97:7721-7726]. The reaction requires the presence of a protelomerase target site telRL on the DNA molecule. This telomere resolution site is a 56-bp imperfect palindrome that consists of the two halves telR and telL and contains a central perfect palindrome telO that binds the protelomerase. This protelomerase is used to produce DNA fragments with covalently closed ends. The gene encoding EGFP can be used. It is present on a circular plasmid DNA, which also harbored a telRL site upstream of the CMV promoter. The presence of the protelomerase target site allows the conversion of the circular covalently closed coiled plasmid DNA (ccc-DNA) to a linear covalently closed DNA molecule by exposure to purified TelN. The linear covalently closed DNA molecules lc-DNA and the molecules linearized by a restriction endonuclease linear open (10) DNA can be designated. The linear covalently closed DNA functions as a template for transcription in mammalian cells. The linearization of the plasmid DNA by TelN and XhoI is demonstrated by agarose gel electrophoresis. As a control, ccc-DNA is used, which has been subjected to the same procedure without DNA-modifying enzymes. Equal amounts of ccc-DNA, lc-DNA formed as described above or by Example 2, and lo-DNA are introduced into HEK293 cells by transient transfection using lipofectamine. The linear covalently closed DNA can yield comparable expression levels of EGFP as the parental ccc-DNA, whereas the linear covalently closed DNA expressed significantly less EGFP. Similar results are obtained by transient calcium phosphate transfection. GFP activity can also be viewed by fluorescence microscopy. Since this method is not easily quantifiable, Western blot analysis can be used. This is expected to demonstrate that the TelN-derived linear DNA molecule is a functional template.

Linear Closed Mini DNA Derived by the TelN/telRL System Using Two telRL Sites.

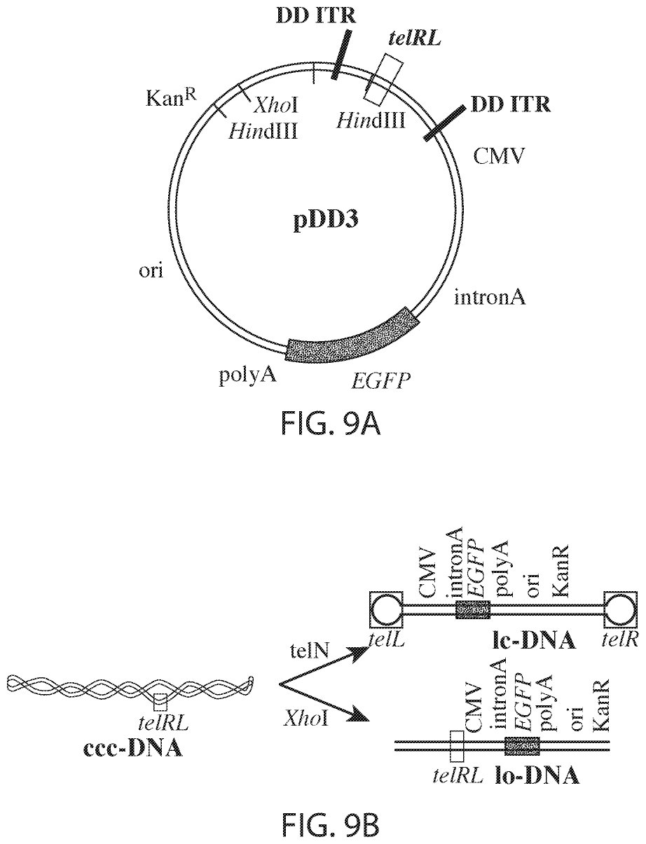

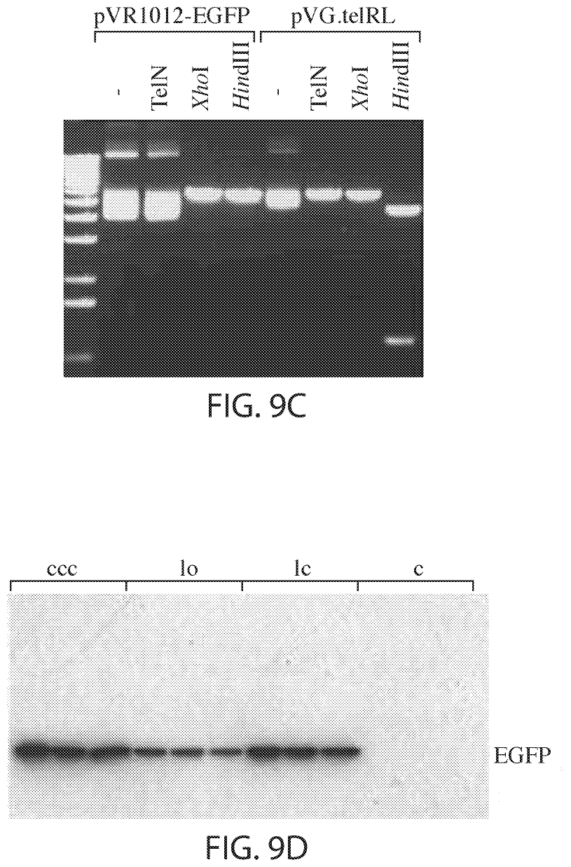

A second telRL site can be inserted downstream of the polyadenylation signal in the DD-ITR containing expression plasmid. In these constructs the transcriptional unit is flanked by telRL at both sites. The linear closed DNA molecule excised by TelN from the parental plasmid is designated mini covalently closed DNA. It contains the viral CMV promoter, the intron A sequence, the gene of interest, and the polyadenylation site, and flanking DD-ITRs and lacks the origin of replication and the antibiotic resistance gene (FIG. 9A). The mini lc-DNA is compared with its equivalent linear open form (mini lo-DNA). Mini lc- and lo-DNAs can be derived from the EGFP-expressing plasmid by digestion with TelN and MunI, respectively. Equimolar amounts of the mini DNAs and the ccc-DNA are used to transiently transfect HEK293 cells using lipofectamine (FIG. 9B). An empty vector DNA can be used as control. A second plasmid encoding a C-terminal fragment of connector enhancer of kinase suppressor of Ras (CNK) fused to EGFP is cotransfected to control for equal transfection efficiencies. Expression of EGFP from the mini lc-DNA is similar to the ccc-DNA, but substantially higher than from mini lo-DNA (FIG. 9B).

Methods for producing DNA vectors described herein generated by the prokaryotic cleaving-joining enzyme TelN can be further reviewed in, e.g., Heinrich, J., et al. J Mol Med. (2002) 80: 648-654, and U.S. Pat. No. 9,290,778, the contents of which are incorporated herein by reference in their entireties.

### Example 4: A Protocol for AAV Particle Production and Purification

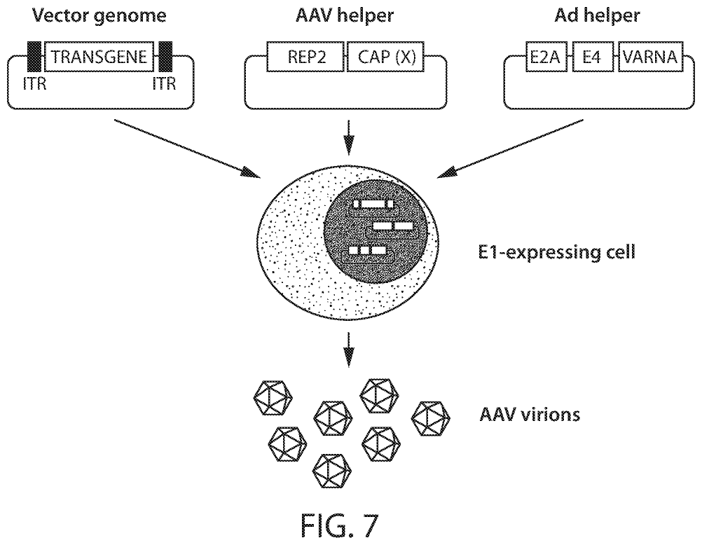

The covalently closed linear DNA vector of Examples 1, 2, and 3 are used as a rAAV vector genome template for production of rAAV particles by the technique described in Mizukami et al. (1998). A Protocol for AAV vector production and purification (Doctoral dissertation, Division of Genetic Therapeutics, Center for Molecular Medicine, Jichi Medical School). See FIG. 7.

A helper plasmid that encodes the E2A, E4, and VA regions (Ad-helper plasmid) is used to transfect 293 cells of human embryonic kidney cells encodes the E1 region of the Ad5 genome, together with the closed DNA of Example 1 (vector plasmid), as well as the rep and cap genes (AAV-helper plasmid).

Reagents
Helper plasmid DNA (pHLP, pAdeno)
Closed linear DNA containing the transgene flanked by DD-ITRs
293 cells (human embryonic kidney cells)
DMEM/F12 culture medium
Fetal bovine serum
2×HBS buffer, containing 290 mM NaCl, 50 mM HEPES buffer and 1.5 mM Na2HPO4, pH 7.1
300 mM CaCl2
Phosphate-buffered saline (PBS)
1 M HEPES buffer, pH 7.4
100 mM Tris-HCl (pH 8.0) plus 150 mM NaCl (TBS)
0.5 M EDTA (pH 8.0)
40% sucrose plus 0.01% BSA in TBS
DNase buffer, containing 50 mM Hepes (pH 7.6), 0.15 M NaCl and 10 mM
MgCl₂
HNE buffer, containing 50 mM Hepes (pH 7.4), 0.15 M NaCl and 25 mM
EDTA
A solution of CsCl in HNE (1.25 g/ml) in HNE
A solution of CsCl in HNE (1.50 g/ml) in HNE

Plasmids

The AAV helper plasmid pHLP, harboring rep and cap, has been described previously as pHLP19. The Ad-helper plasmid pAdeno is identical to pVAE2AE4-5 and encodes the entire E2A and E4 regions plus the VA RNA I and II genes.

Transfection and Extraction

Trypsinized 293 cells at 5×10⁶ cells per 225-cm² flask are used to generate a monolayer of 20% to 40% confluence when cells attach initially to the surface of the flask. Use 40 ml of medium per flask The plate is placed in an incubator in 5% CO₂ in air and cells are grown to 80% confluence (24 to 48 h.).

One hour before transfection, half the medium in the flask is replaced with fresh medium. 23 μg of the closed linear DNA vector containing DD-ITRs and of each helper plasmid to 4 ml of 300 mM CaCl₂. This solution is gently added to 4 ml of 2×HBS and mixed immediately by gentle inversion three times. This mixture is immediately pipetted into the 225-cm² flask of 293 cells in 40 ml of DMEM/F12 medium plus 10% FCS and is swirled to produce a homogeneous solution. The plate is immediately returned to the incubator and incubated at 37° C. for 4 to 6 hr. Do not disturb the plate during this period. At the end of the incubation, the medium is replaced with pre-warmed DMEM/F12 culture (contains 2% FCS). Three days after transfection, 1 ml of 0.5 M EDTA is added to the flask and incubated for 3 min at room temperature. The suspension of cells is collected and is centrifuged at 300×g for 10 min. The supernatant is removed and the cells in the pellet resuspended in 2 ml of TBS.

Freeze and thaw the cells that have been suspended in TBS three times by placing them alternately in a dry ice/ethanol bath until the suspension is completely frozen and in a water bath of 37° C. until it is completely thawed. Return the sample to the ice bath immediately when it is completely thawed. Remove tissue debris by centrifugation at 10,000×g for 10 min and collect the supernatant.

Purification of the AAV Vector

Prepare supernatants, as described above from 24 flasks. Place 11 ml of a solution of 40% sucrose plus 0.01% BSA in TBS in a sterile ultracentrifuge tube (Ultrabottle #3430-3870; Nalge Nunc, Rochester, N.Y.). Carefully overlay the 48 ml of pooled supernatants on this solution. Pellet the crude viral particles by centrifugation at 100,000×g for 16 hr at 4° C. Resuspend the pellet by vigorous agitation in 5 ml of DNase buffer. Add 1,000 units of DNase I and incubate for 1 hr at 37° C. Add 250 μL of 0.5 M EDTA and then remove debris by centrifugation at 10,000×g for 2 min. Then filter the supernatant through a low-protein-binding 5-μm syringe filter (Sterile Acrodisc Syringe Filter; Pall Gelman Laboratory, Ann Arbor, Mich.). Load the filtered material onto a two-tier CsCl gradient (1.25 g/ml and 1.50 g/ml) prepared in HNE buffer. Spin the gradient at 35,000 rpm for 2 h at 16° C. in an SW40 rotor (Beckman Instruments, Palo Alto, Calif.). Collect the band of viral particles and load it on a second two-tier CsCl gradient (1.25 g/ml and 1.50 g/ml) prepared in HNE buffer. Spin the gradient at 65,000 rpm for 2 h at 16° C. in a VTi65.2 rotor (Beckman Instruments). Fractions of 0.5 ml each are collected and the virus-rich fraction is selected by semi-quantitative PCR analysis, Western blotting with antibodies against Cap, or quantitative DNA dot-blot hybridization. A dialysis cassette (Slide-A-Lyzer; Pierce, Rockford, Ill.) is used to desalt the virus-rich fraction by three cycles of dilution with 300 ml of HNE buffer. The material is concentrated to 50 μL with Ultrafree-4 (Millipore, Bedford, Mass.) according to the manufacturer's instruction. The final titer usually ranges between 1×10¹³ and 5×10¹³ particles from 5×10⁸ 293 cells, as determined by quantitative DNA dot-blot hybridization or Southern blotting.

The above method is repeated except the closed linear DNA does not have the double D and is used as a comparator. The double DD containing closed linear circle is expected to be more efficient—it has a higher yield of packaged genome infectious viral particles.

### Example 5: Delivery of the Vector to Target Cells

The resultant closed end vectors of Example 1 containing the luciferase reporter gene are delivered to target cells by transfection. Constructs with no spacers, and with spacers of 2, 5, 10, 20 and 25 nucleotides generated in Example 1 are used delivered to HeLa cells to verify delivery of the vector, and expression of expression constructs (luciferase reporter gene) from covalently closed linear DNA produced in Example 1. Luciferase expression from cells transfected with constructs with no spacers, and with spacers of 2, 5, 10, 20 and 25 nucleotides is quantitatively detected by luciferase assay. Plasmid DNA and covalently closed circular DNA (e.g. containing the same expression construct), and open linear DNA of the same type, e.g. synthetically produced by cleavage of unique restriction sites to cleave the hairpin ends, are used as controls. Transfection is carried out at a 60% confluence in 20 mm diameter wells in RPMI using Transfectam™ (Promega) in accordance with manufacturer's instructions. Each transfection uses 400 ng of construct DNA. Transfection frequency is normalised within and between experiments by inclusion of an internal control using 40 ng of the Renilla luciferase-expressing plasmid pGL4.73 (containing the hRluc gene from Renilla reniformis) in each transfection. Firefly luciferase (luminescence from Photinus pyralis) and Renilla luciferase activity is measured sequentially using the Dual-Luciferase™ Reporter (DLR™) Assay System (Promega). Relative light units are measured using a GloMax Multi Luminometer (Promega) and results are expressed as the ratio of Firefly luciferase/Renilla luciferase. All experiments are carried out in triplicate.

Results are expected to indicate that closed linear DNA or particles containing DD-ITRs, including that amplified by RCA, expresses luciferase at higher levels over time than the open linear PCR constructs and open linear PCR constructs lacking the DD-ITR. This will demonstrate that closed linear DNA produced in accordance with the invention may be used to successfully express luciferase when introduced into mammalian cells. In addition, the closed linear DNA vectors are expected to persist longer within the cell as compared to the controls. Further, DNA that have spacers between the DD-ITR and the promoter are expected to generate higher expression of luciferase than otherwise identical constructs without spacers. The size of the spacers is expected to correlate with expression levels, with larger spacers (e.g. 20-25 nucleotide spacers) leading to higher expression.

In addition, it is expected that the closed linear DNA vectors can form concatemers in vivo. These can form either as extrachromosomal DNA or integrated into the cellular genome. For example, they can exist randomly head to head, tail to tail, head to tail. Assays for concatemers are performed by methods known in the art (Chen et al., (2001) Molecular Therapy 3: 403-410; Wilson et al., (1982) Mol. Cell. Biol. 2: 1258-1269; Folger et al., (1982) Mol. Cell. Biol. 2: 1372-1387; Critchlow et al., (1998) Trends Biochem. Sci. 23: 394-398; Wu et al., (1999) PNAS USA 96: 1303-1308).

### Example 6—the Vector with Expression Cassette Persists in Recipient Tissue Cells

In order to demonstrate clinical relevance, an expression cassette containing the human factor IX minigene driven by the ApoE enhancer/HCR and al-antitrypsin promoter (Miao et al., (2000) in vitro. Mol. Ther. 1:522-532) is delivered to the liver of hemophilia B mice using different vectors (the closed linear DNA comprising the DD-ITR and telomeric ends of the instant invention, corresponding linear DNA, and corresponding plasmid DNA. The mice are analyzed for vector presence and for gene expression of factor IX by analysis of serum human factor IX concentrations at various times following the injections (3, 4, 5, 6, 7, 8, 9, 10 weeks, and at 3 months, 5 months, 10 months, over a year, etc).

The recipients of the DD-ITR constructs containing the factor IX expression cassette are expected to have higher amounts of vector present over time, and have higher concentrations of the factor IX than recipients of ss linear DNA and plasmid DNA controls. The amount of vector present and the concentrations of the human factor IX is expected to persist over time in the recipients of the DD-ITR constructs, and to decrease over time in the recipients of the controls. In addition, over time, the expression of the factor IX in the DD-ITR construct in recipient mice is expected to persist at substantially higher levels and over a substantially longer period of time than expression of the Factor IX in recipients of the controls. This is determined by quantitation of human factor IX in serum by ELISA analysis, and identification of correction of their bleeding diathesis, and also by quantitation of vector in the livers of treated mice by Southern blot analysis at different times following vector administration. In addition, the number of hepatic nuclei containing the vectors in the treated mice are determined by in situ hybridization of liver sections, to verify that the relative number of hepatic nucleic containing the vectors is similar.

Molecular Structure of Delivered DNA In Vivo

The vector is expected to form concatemers in recipient tissue cells that persist over time. The concatemers persist extrachromasomally, or integrate into the host cell genome. To demonstrate this, recipient mice liver tissue is analyzed by Southern blot analysis for the molecular structure of the vector DNA. The DNA is isolated and then digested with a restriction endonuclease that either does not cut within the vector, or cuts once in the expression cassette. Analysis of the cut DNA is performed to determine integration of DNA into the mouse genome or retention extrachromasomally. The production of a high molecular weight band in all samples from digestion with an endonuclease that fails to cut within the vector DNA, is consistent with either the integration of the DNA vector into the mouse genome or rapid formation of concatemers in vivo. These two possibilities are distinguished by digestion of the liver DNA samples with the restriction endonuclease that cuts once through the expression cassette. Conversion of the high molecular DNA signal into a DNA ladder by this digestion would indicate concatemerization in vivo.

We expect that linkage of the DNA fragments occurs randomly in a head-to-head, tail-to-tail, and head-to-tail orientation. This can be verified by appropriate digestion of the DNA samples with restriction enzymes and analysis/probing of the resulting fragments.

The majority of the high-molecular-weight DNA may originate from concatemers. However, to determine if this is extrachromasomal or integrated into the genome, the mice are injected with either the DD-ITR vector DNA, or an integrating factor IX transposon (Yant et al., Nat. Genet. 25: 35-41) to a ⅔ partial hepatectomy. In the transposon group, the transposase, expressed from one plasmid, mediates the release of the human factor IX expression cassette flanked by the transposon ITR from a second plasmid and the insertion of the released transgene expression cassette into the mouse genome. If the partial hepatectomy results in one or two rounds of hepatocyte cell division and a significant loss of extrachromosomal DNA, this would indicate that in contrast to integrating plasmid-infused mice, whose transgene expression should be unchanged by the induction of hepatocyte proliferation, the vector DNA treated mice will show a 10-fold drop in gene expression after partial hepatectomy over the same time period. These data would indicate that transcriptionally active DD-ITR vectors remain predominantly extrachromosomal in the liver. The alternative results would indicate that the active DD-ITR vectors are integrated into the liver cell chromosomes.

Methods

Animal studies. Eight- to ten-week-old female C57BL/6 mice are obtained from Taconic Farms, Inc. (Germantown, N.Y.). All animal procedures are performed under the guidelines set forth by Stanford University and the National Institute of Health. Forty micrograms of DNA in 2 ml 0.85% saline is injected into mouse tail veins as previously described (Liu et al., (1999) Gene Ther. 6: 1258-1266; Zhang et al., (1999) Human Gene Therapy 10: 1735-1737). For each injection, the mass of DNA is the same while the molar ratio can vary (e.g., by twofold). In other studies, small variations in molar ratios is not expected to significantly affect gene expression. Mice are bled periodically by a retro-orbital technique. In some cases, mice are subjected to a surgical ⅔ partial hepatectomy as previously described (Park et al., Nat. Genet. 24: 49-52 (2000)). The bleeding times in mice are determined by measuring the time required for clotting of the blood from a 2- to 3-mm tail snip, as previously described (Yant et al., (2000) Nat. Genet. 25: 35-41).

In situ hybridization. Five-micrometer sections of paraffin-embedded livers of the mice 2 to 3 weeks after receiving 40 μg of the respective construct DNA via tail vein infusion are processed for in situ hybridization according to the protocol described previously (Miao et al. (2000) J. Virol. 74: 3793-3803). Following deparaffinization, rehydration, denaturation, and digestion with proteinase, the sections are incubated with a denatured DNA probe specific for the vector labeled with digoxigenin using the DIG labeling kit from Roche Molecular Biochemicals (Indianapolis, Ind.). After hybridization, the sections are incubated with a goat anti-digoxigenin antibody conjugated with alkaline phosphatase, and the alkaline phosphatase-bound vector DNA is visualized by nitroblue tetrazolium chloride-5-4-chloro-3-indolyl phosphate (Roche Molecular Biochemicals).

ELISA quantitation. After DNA delivery, mouse blood is collected periodically, and human factor IX (hFIX) (Walter et al., (1996) PNAS USA 93: 3056-3061) is quantitated by ELISA.

Southern blot analyses. Mice are sacrificed at period times after DNA injection and total liver DNA is prepared by a salting-out procedure. Twenty micrograms of liver DNA is digested with restriction enzymes, separated by gel electrophoresis, and analyzed by Southern blot hybridization using a cDNA as a probe. Radioactive DNA bands are quantitated by phosphoimager analyses.

The present invention is not to be limited in scope by the exemplified embodiments disclosed herein which are intended as illustrations of single aspects of the invention, and any clones, DNA or amino acid sequences which are functionally equivalent are within the scope of the invention. Indeed, various modifications of the invention, in addition to those shown and described herein, will become apparent to those skilled in the art from the foregoing description and accompanying drawings. Such modifications are intended to fall within the scope of the appended claims.

### Example 7—Making Templates that Produce Circular Nucleic Acids

Exemplified herein are methods for making a template useful for generating vectors for expressing cystic fibrosis transmembrane conductance regulator (CFTR) in a subject. A plasmid having, from 5′ to 3′, a BAMHI restriction site, a F1 ORI, a PvuII restriction site, an ITR-L, a promoter operatively linked to the coding region of CFTR, a Double D-ITR (DD-ITR), a promoter operatively linked to the coding region of CFTR, ITR-R, and a HINDIII restriction site, is digested with BAMHI and HINDIII restriction enzymes for 24 hours at 37° C. The digest is run on an electrophoresis gel to visual and isolate the plasmid fragment. The plasmid fragment is cut out of the gel and purified. An adaptor sequence having BAMHI restriction site sequence at the end of a hairpin loop and an adaptor sequence having a HINDIII restriction site sequence at the end of a hairpin loop are additionally digested and purified in the same manner.

To form the circular nucleic acid template, the adaptor sequences are annealed to the cut ends of the plasmid fragment. The purified plasmid fragment and adapter sequences are ligated in the presence of a ligase, such as T4 ligase, and ATP at room temperature for at least 1 hour. The ligation reaction is then heat inactivated at 65° C. for 10 minutes to inactivate the ligase enzyme.

The circular nucleic template acid is transformed into E. coli cells and grown at 37° C., shaking, for 14-16 hours to induce replication of the circular nucleic acid. The circular nucleic acid encoding the CFTR transgene is released from E. coli cells using a bacterial lysis reagent to cause cell lysis. Following release, the CFTR circular nucleic acid is recovered using standard methods, for example, via purification using column chromatography. The circular nucleic acid can be recovered and used directly for delivery of the transgene in vivo, or used for viral production (see Example 8).

The recovered CFTR circular nucleic acid is further digested with a PvuII restriction enzyme for 24 hours at 25° C. to cut the PvuII cleavage site. Cutting PvuII removes the ORI and creates an open-end on the CFTR nucleic acid construct. The open ended circular nucleic acid can be used for in vivo delivery of transgene or for production of recombinant viral DNA. The circular nucleic acid need not be digested with PvuII to be used for recombinant viral production, or for in vivo delivery of transgene.

### Example 8: Manufacturing of Viral Vectors Using Circular Nucleic Acids

The open- and closed-ended CFTR nucleic acid construct is used to manufacture viral vectors in Pro10/HEK293 cells. Pro10/HEK293 cells, as described in U.S. Pat. No. 9,441,206, are ideal for scalable production of AAV vectors. This cell line is contacted with the CFTR nucleic acid construct via transfection to express the circular nucleic acid. Expression of CFTR nucleic acid construct is confirmed via PCR-based assays using primers specific for the plasmid.

Transfection. Pro10/HEK293 cells are transfected with CFTR circular nucleic acid and are also transfected with a Packaging plasmid encoding Rep2 and serotype-specific Cap2: AAV-Rep/Cap, and the Ad-Helper plasmid (XX680: encoding adenoviral helper sequences).

On the day of transfection, the cells are counted using a ViCell XR Viability Analyzer (Beckman Coulter) and diluted for transfection. To mix the transfection cocktail the following reagents are added to a conical tube in this order: plasmid DNA, OPTIMEM® I (Gibco) or OptiPro SFM (Gibco), or other serum free compatible transfection media, and then the transfection reagent at a specific ratio to plasmid DNA. The cocktail is inverted to mix prior to being incubated at room temperature. The transfection cocktail is pipetted into the flasks and placed back in the shaker/incubator. All optimization studies are carried out at 30 mL culture volumes followed by validation at larger culture volumes. Cells are harvested 48 hours post-transfection.

Production of rAAV Using Wave Bioreactor Systems. Wave bags are seeded 2 days prior to transfection. Two days post-seeding the wave bag, cell culture counts are taken and the cell culture is then expanded/diluted before so transfection. The wave bioreactor cell culture is then transfected. Cell culture are harvested from the wave bio-reactor bag at least 48 hours post-transfection.

Titer: AAV titers are calculated after DNase digestion using qPCR against a standard curve (AAV ITR specific) and primers specific to the CFTR circular nucleic acid.

Harvesting Suspension Cells from Shaker Flasks and 60 Wave Bioreactor Bags. 48 hours post-transfection, cell cultures are collected into 500 mL polypropylene conical tubes (Corning) either by pouring from shaker flasks or pumping from wave bioreactor bags. The cell cultures are then centrifuged at 655×g for 10 min using a Sorvall RC3C plus centrifuge and H6000A rotor. The supernatant is discarded, and the cells are resuspended in 1×PBS, transferred to a 50 mL conical tube, and centrifuged at 655×g for 10 mM. At this point, the pellet could either be stored in NLT-60° C. or continued through purification.

Titering rAAV from Cell Lysate Using qPCR. 10 mL of cell culture is removed and centrifuged at 655×g for 10 min using a Sorvall RC3C plus centrifuge and H6000A rotor. The supernatant is decanted from the cell pellet. The cell pellet is then resuspended in 5 mL of DNase buffer (5 mM CaCl2, 5 mM MgCl2, 50 mM Tris-HCl pH 8.0) followed by sonication to lyse the cells efficiently. 300 uL is then removed and placed into a 1.5 mL microfuge tube. 140 units of DNase I is then added to each sample and incubated at 37° C. for 1 hour. To determine the effectiveness of the DNase digestion, 4-5 mg of TReGFP plasmid is spiked into a non-transfected cell lysate with is and without the addition of DNase. 504, of EDTA/Sarkosyl solution (6.3% sarkosyl, 62.5 mM EDTA pH 8.0) is added to each tube and incubated at 70° C. for 20 minutes. 50 μL of Proteinase K (10 mg/mL) is then added and incubated at 55° C. for at least 2 hours. Samples are boiled for 15 minutes to inactivate the Proteinase K. An aliquot is removed from each sample to be analyzed by qPCR. Two qPCR reactions are carried out in order to effectively determine how much rAAV vector is generated per cell. One qPCR reaction is set up using a set of primers 2s designed to bind to a homologous sequence on the backbones of plasmids XX680, pXR2 and TReGFP. The second qPCR reaction is set up using a set of primers to bind and amplify a region within the eGFP gene. qPCR is conducted using Sybr green reagents and Light cycler 480 from 30 Roche. Samples are denatured at 95° C. for 10 minutes followed by 45 cycles (90° C. for 10 sec, 62° C. for 10 sec and 72° C. for 10 sec) and melting curve (1 cycle 99° C. for 30 sec, 65° C. for 1 minute continuous).

Purification of rAAV from Crude Lysate. Each cell pellet is adjusted to a final volume of 10 mL. The pellets are vortexed briefly and sonicated for 4 minutes at 30% yield in one second on, one second off bursts. After sonication, 550 U of DNase is added and incubated at 37° C. for 45 minutes. The pellets are then centrifuged at 9400×g using the Sorvall RCSB centrifuge and HS-4 rotor to pellet the cell debris and the clarified lysate is transferred to a Type70Ti centrifuge tube (Beckman 361625). In regard to harvesting and lysing the suspension HEK293 cells for isolation of rAAV, one skilled in the art can use as mechanical methods such as microfluidization or chemical methods such as detergents, etc., followed by a clarification step using depth filtration or Tangential Flow Filtration (TFF).

AAV Vector Purification. Clarified AAV lysate is purified by column chromatography methods as one skilled in the art would be aware of and described in the following manuscripts (Allay et al., Davidoff et al., Kaludov et al., Zolotukhin et al., Zolotukin et al, etc), which are incorporated herein by reference in their entireties.

What is claimed:

1. A method for introducing a nucleic acid construct into a target cell for sustained expression comprising administering to the target cell a covalently closed non-viral DNA construct comprising:
a. at least one DD-ITR comprising:
i. an inverted terminal repeat having an A, A′, B, B′, C, C′ and D region;
ii. a D′ region;
iii. wherein the D and D′ region are complementary palindromic sequences, and wherein D and D′ are positioned adjacent the A and A′ region;
b. complementary strands of the nucleic acid construct comprising a predetermined DNA sequence that can anneal into expressible dsDNA;
c. wherein the DNA construct forms linear DNA with covalently closed hairpin ends; and
d. wherein the DNA construct can express the predetermined DNA sequence in the target cell.

2. The method of claim 1, wherein the D regions contain a nicking site.

3. The method of claim 1, wherein the D regions are at least 5 nucleotides in length.

4. The method of claim 1, wherein the D regions are about 20 nt in length.

5. The method of claim 1, wherein the D region corresponds to a parvovirus D region of a parvovirus ITR.

6. The method of claim 1, wherein the parvovirus is a dependovirus.

7. The method of claim 1, wherein the dependovirus is AAV.

8. The method of claim 1, wherein the predetermined DNA sequence is operably linked to a promoter.

9. The method of claim 8, wherein the ITR is acting as a promoter.

10. The method of claim 8, wherein the promoter is separate from the ITR.

11. The method of claim 1-10, wherein the DD-ITR drives expression of the predetermined DNA sequence.

12. The method of claim 4, wherein the D and D′ region has a substitution, insertion, and/or deletion that retains at least 5 nucleic acids of the region.

13. The method of claim 12, wherein the retained nucleic acids comprise the nicking site and/or junction of the A and A′ region and the D and D′ regions.

14. The method of claim 1, wherein the predetermined DNA sequence encodes a protein, a protein fragment, a peptide, or a functional RNA.

15. The method of claim 14, wherein the functional RNA is selected from the group consisting of micro RNA, RNAi, shRNA, and guide RNA for Crisper Cas 9 recombination.

16. The method of any of claims 1-15, wherein there are at least 2 nucleotides between the D and D′ region and the predetermined DNA sequence as spacers.

17. The method of claim 14, wherein there are at least 2 nucleotides between the D and D′ region and the promoter as spacers.

18. The method of claim 16 or 17, wherein the spacers are at least 5 nucleotides.

19. The method of claim 16 or 17, wherein the spacers are at least 20 nucleotides.

20. The method of claim 16 or 17, wherein the spacers are at least 25 nucleotides.

21. The method of any one of claims 1-20, wherein the at least one DD-ITR is generated from an AAV ITR, a parvovirus ITR, or a synthetic ITR.

22. The method of any one of claims 1-21, wherein the DNA construct comprises two DD-ITRs.

23. The method of any one of claims 1-22, wherein the D regions are from different stereotypes than the ITR.

24. The method of claim 22, wherein each DD-ITR is derived from a different viral serotype.

25. The method of claim 22 wherein one DD-ITR is derived from an AAV2 ITR, and a second DD-ITR is derived from an AAV5 ITR.

26. The method of any one of claims 1-25, wherein there is a deletion, substitution and/or insertion in the B and B′ or C and C′ region.

27. The method of any one of claims 1-26, wherein there is a deletion, substitution and/or insertion in the A and A′ region.

28. The method of any one of claims 1-27, wherein the DNA construct further comprises a partial protelomerase binding site at the covalently closed ends formed by protelomerase enzyme activity in a host cell.

29. The method of claim 28, wherein the host cell expresses the protelomerase under the control of an inducible promoter.

30. The method of any one of claims 1-27, wherein the DNA construct further comprises a partial protelomerase binding site at the covalently closed ends formed by protelomerase enzyme activity in vitro.

31. The method of any one of claims 1-30, wherein the DNA construct persists within the target cell and results in sustained expression of the predetermined sequence.

32. The method of any one of claims 1-31, wherein the DNA construct can be converted into a concatemeric structure in the cell.

33. The method of any one of claims 1-32, wherein the sustained expression of the predetermined DNA sequence in the target cell is for a period of time at least at least 2-5 weeks, at least 1-12 months, at least 1-10 years.

34. The method of any one of claims 32-33, wherein the concatemeric structure persists in the target cell and results in sustained expression of the predetermined sequence.

35. The method of any one of claims 32-34, wherein the concatemeric structure persists in the target cell extra-chromosomally.

36. The method of any one of claims 32-34, wherein the concatemeric structure integrates into the target cell chromosome.

37. The method of any one of claims 1-36 wherein nucleic acid is a therapeutic nucleic acid.

38. The method of any one of claims 1-37, wherein the target cell is in vitro.

39. The method of any one of claims 1-37 wherein the target cell is in vivo.

40. The method of any one of claims 1-37, wherein the construct is administered to the target cell ex vivo.

41. The method of any one of claims 1-40, wherein the target cell is a genetically deficient cell and/or a diseased cell.

42. The method of any one of claims 1-41, wherein the target cell is a diseased cell.

43. The method of any one of claims 1-42, wherein the target cell is selected from the group consisting of a neural cell, lung cell, retinal cell, epithelial cell, smooth muscle cell, skeletal muscle cell cardiac muscle cell, pancreatic cell, hepatic cell, kidney cell, myocardial cell, bone cell, spleen cell, keratinocyte, fibroblast, endothelial cell, prostate cell, germ cell, progenitor cell, stem cell, cancer cell, and tumor cell.

44. A DNA vector for delivery of a predetermined nucleic acid sequence into a target cell for sustained expression, comprising,
c. two DD-ITRs each comprising:
i. an inverted terminal repeat having an A, A′, B, B′, C, C′ and D region;
ii. a D′ region; and
iii. wherein the D and D′ region are complementary palindromic sequences of about 5-20 nt in length, are positioned adjacent the A and A′ region;
d. the predetermined nucleic acid sequence (e.g. a heterologous gene for expression); and
wherein the two DD-ITRs flank the nucleic acid in the context of covalently closed non-viral DNA.

45. The DNA vector of claim 44, wherein the predetermined nucleic acid sequence is operably linked to a promoter.

46. The DNA vector of claim 44, wherein the DD-ITR drives expression of the predetermined nucleic acid sequence.

47. The DNA vector of claim 46, wherein the D and D′ region has a substitution, insertion, and/or deletion that retains at least 5 nucleic acids of the region.

48. The DNA vector of claim 47, wherein the retained nucleic acids comprise the nicking site and/or junction of the A and A′ region and the D and D′ regions.

49. The DNA vector of claim 44, wherein the predetermined nucleic acid sequence encodes a protein, a protein fragment, a peptide, or a functional RNA.

50. The DNA vector of claim 49, wherein the functional RNA is selected from the group consisting of micro RNA, RNAi, shRNA, and guide RNA for Crisper Cas 9 recombination.

51. The DNA vector of claim 45, wherein there are at least 2 nucleotides between the D and D′ region and the predetermined nucleic acid sequence as spacers.

52. The DNA vector of claim 46, wherein there are at least 2 nucleotides between the D and D′ region and the promoter as spacers.

53. The DNA vector of claim 51 or 52 wherein the spacers are at least 5 nucleotides.

54. The DNA vector of claim 53, wherein the spacers are at least 20 nucleotides.

55. The DNA vector of claim 53, wherein the spacers are at least 25 nucleotides.

56. The DNA vector of any one of claims 44-55, wherein the DD-ITRs are generated from an ITR selected from the group consisting of a parvovirus ITR, and a synthetic ITR.

57. The DNA vector of claim 56, wherein the parvovirus is a dependovirus.

58. The DNA vector of claim 57, wherein the dependovirus is AAV.

59. The DNA vector of any one of claims 44-58, wherein the DNA construct comprises more than two DD-ITRs.

60. The DNA vector of claim 59, wherein each DD-ITR is derived from a different viral serotype.

61. The DNA vector of claim 60, wherein one DD-ITR is derived from an AAV2 ITR, and a second DD-ITR is derived from an AAV5 ITR.

62. The DNA vector of any one of claims 44-61, wherein there is a deletion, substitution or insertion in the B and B′ or C and C′ region.

63. The DNA vector of any one of claims 44-61, wherein there is a deletion, substitution or insertion in the A and A′ region.

64. The DNA vector of any one of claims 44-63, wherein the DNA vector further comprises a partial protelomerase binding site and wherein the covalently closed ends are formed by protelomerase enzyme activity in vitro.

65. The DNA vector of any one of claims 44-64, wherein the DNA vector persists within the target cell and results in sustained expression of the predetermined sequence.

66. The DNA vector of any one of claims 44-65, wherein the DNA vector can be converted into a concatemeric structure in the cell.

67. The DNA vector of any one of claims 44-66, wherein the sustained expression of the predetermined DNA sequence in the target cell is for a period of time at least 2-5 weeks, at least 1-12 months, at least 1-10 years.

68. The DNA vector of any one of claims 66-67, wherein the concatemeric structure persists within the target cell and results in sustained expression of the predetermined sequence.

69. The DNA vector of any one of claims 66-68, wherein the concatemeric structure persists in the target cell extra-chromosomally.

70. The DNA vector of any one of claims 66-68, wherein the concatemeric structure integrates into the target cell chromosome.

71. The DNA vector of any one of claims 44-70, wherein predetermined nucleic acid is a therapeutic nucleic acid.

72. The DNA vector of claim 44-71 wherein at least one DD-ITR is an AAV ITR.

73. The DNA vector of any one of claims 44-72, wherein the DNA vector further comprises a partial protelomerase binding site flanking the two DD-ITRs.

74. The DNA vector of claim 73, wherein the partial protelomerase binding sites flanking the two DD-ITRs are formed by protelomerase enzyme activity in vitro or by protelomerase enzyme activity in vivo.

75. The DNA vector of any one of claims 44-74, wherein the covalently closed non-viral DNA construct persists as a concatemeric structures within the target cell.

76. The DNA vector of claim 75, wherein the DNA vector promotes sustained expression of the nucleic acid for a period of time from 2-5 weeks, from 1-12 months, from 1-10 years, or longer.

77. A method for introducing a nucleic acid into a target cell for sustained expression comprising administering to the target cell a covalently closed non-viral DNA construct comprising:
a. at least one ITR sequence selected from the group consisting of the ITR's shown in FIG. 5;
b. complementary strands of the nucleic acid construct, wherein the nucleic acid construct comprises a predetermined DNA sequence, wherein the complementary strands can anneal into expressible dsDNA; and
c. wherein the DNA construct forms linear DNA with hairpin covalently closed ends.

78. The method of claim 77, wherein the ITR sequence is flanked on either side by complementary sequences D and D′ to thereby create a DD-ITR.

79. The method of claim 77, wherein the predetermined DNA sequence is operably linked to a promoter.

80. The method of claim 77, wherein the DD-ITR drives expression of the predetermined DNA sequence.

81. The method of claim 77, wherein the D and D′ region has a substitution, insertion, and/or deletion that retains at least 5 nucleic acids of the region.

82. The method of claim 81, wherein the retained nucleic acids comprise the nicking site and/or junction of the A and A′ region and the D and D′ regions.

83. The method of claim 77, wherein the predetermined DNA sequence encodes a protein, a protein fragment, a peptide, or a functional RNA.

84. The method of claim 83, wherein the functional RNA is selected from the group consisting of micro RNA, RNAi, shRNA, and guide RNA for Crisper Cas 9 recombination.

85. The method of claim 77-84 wherein there are at least 2 nucleotides between the D and D′ region and the predetermined DNA sequence as spacers.

86. The method of claim 77-85, wherein there are at least 2 nucleotides between the D and D′ region and the promoter as spacers.

87. The method of claim 85 or 86, wherein the spacers are at least 5 nucleotides.

88. The method of claim 87, wherein the spacers are at least 20 nucleotides.

89. The method of claim 87, wherein the spacers are at least 25 nucleotides.

90. The method of any one of claims 77-89, wherein the at least one DD-ITR is generated from a parvovirus ITR or a synthetic ITR.

91. The method of claim 90 wherein the parvovirus is a dependovirus.

92. The method of claim 91, wherein the dependovirus is AAV.

93. The method of any one of claims 65-78, wherein the DNA construct comprises two DD-ITRs.

94. The method of claim 93, wherein each DD-ITR is derived from a different viral serotype.

95. The method of claim 93, wherein one DD-ITR is derived from an AAV2 ITR, and a second DD-ITR is derived from an AAV5 ITR.

96. The method of any one of claims 77-95, wherein there is a deletion, substitution or insertion in the B and B′ or C and C′ region.

97. The method of any one of claims 77-95, wherein there is a deletion, substitution or insertion in the A and A′ region.

98. The method of any one of claims 77-97, wherein the DNA construct further comprises a partial protelomerase binding site and wherein the covalently closed ends are formed by protelomerase enzyme activity in vitro.

99. The method of any one of claims 77-98, wherein the DNA construct persists within the target cell and results in sustained expression of the predetermined sequence.

100. The method of any one of claims 77-99, wherein the DNA construct can be converted into a concatemeric structure in the cell.

101. The method of any one of claims 77-100, wherein the sustained expression of the predetermined DNA sequence in the target cell is for a period of time at least 1-5 weeks, at least 2-5 weeks, at least 1-12 months, at least 1-10 years.

102. The method of any one of claims 100-101, wherein the concatemeric structure persists within the target cell and results in sustained expression of the predetermined sequence.

103. The method of any one of claims 100-102, wherein the concatemeric structure persists in the target cell extra-chromosomally.

104. The method of any one of claims 100-102, wherein the concatemeric structure integrates into the target cell chromosome.

105. The method of any one of claims 77-104, wherein nucleic acid is a therapeutic nucleic acid.

106. The method of any one of claims 77-105, wherein the target cell is in vitro.

107. The method of any one of claims 77-105, wherein the target cell is in vivo.

108. The method of any one of claims 77-105, wherein the construct is administered to the target cell ex vivo.

109. The method of any one of claims 77-108, wherein the target cell is a genetically deficient cell.

110. The method of any one of claims 77-108, wherein the target cell is a diseased cell.

111. The method of any one of claims 77-110, wherein the target cell is selected from the group consisting of a neural cell, lung cell, retinal cell, epithelial cell, smooth muscle cell, skeletal muscle cell cardiac muscle cell, pancreatic cell, hepatic cell, kidney cell, myocardial cell, bone cell, spleen cell, keratinocyte, fibroblast, endothelial cell, prostate cell, germ cell, progenitor cell, stem cell, cancer cell, and tumor cell.

112. A cell or population thereof, produced by the method of any one of claim 1-43, or 77-111.

113. A covalently closed non-viral linear DNA vector for delivery of predetermined nucleic acid into a target cell for sustained expression comprising
a. at least one DD-ITR comprising:
i. an inverted terminal repeat having an A, A′, B, B′, C, C′ and D region;
ii. a D′ region;
iii. wherein the D and D′ region are complementary palindromic sequences, and wherein D and D′ are positioned adjacent the A and A′ region;
b. complementary strands of the nucleic acid construct comprising a predetermined DNA sequence that can anneal into expressible dsDNA;
c. wherein the DNA vector construct forms linear DNA with covalently closed hairpin ends; and
d. wherein the DNA vector construct can express the predetermined DNA sequence in the target cell.

114. The DNA vector of claim 113, wherein the D regions contain a nicking site.

115. The DNA vector of claim 113, wherein the D regions are at least 5 nucleotides in length.

116. The DNA vector of claim 113, wherein the D regions are about 20 nt in length.

117. The DNA vector of claim 113, wherein the D region corresponds to a parvovirus D region of a parvovirus ITR.

118. The DNA vector of claim 113, wherein the parvovirus is a dependovirus.

119. The DNA vector of claim 113, wherein the dependovirus is AAV.

120. The DNA vector of claim 113, wherein the predetermined DNA sequence is operably linked to a promoter.

121. The DNA vector of claim 120, wherein the ITR is acting as a promoter.

122. The DNA vector of claim 120, wherein the promoter is separate from the ITR

123. The DNA vector of claim 113-122, wherein the DD-ITR drives expression of the predetermined DNA sequence.

124. The DNA vector of claim 113, wherein the D and D′ region has a substitution, insertion, and/or deletion that retains at least 5 nucleic acids of the region.

125. The DNA vector of claim 124, wherein the retained nucleic acids comprise the nicking site and/or junction of the A and A′ region and the D and D′ regions.

126. The DNA vector of claim 113, wherein the predetermined DNA sequence encodes a protein, a protein fragment, a peptide, or a functional RNA.

127. The DNA vector of claim 126, wherein the functional RNA is selected from the group consisting of micro RNA, RNAi, shRNA, and guide RNA for Crisper Cas 9 recombination.

128. The DNA vector of any of claims 1-127, wherein there are at least 2 nucleotides between the D and D′ region and the predetermined DNA sequence as spacers.

129. The DNA vector of claim 126, wherein there are at least 2 nucleotides between the D and D′ region and the promoter as spacers.

130. The DNA vector of claim 128 or 129, wherein the spacers are at least 5 nucleotides.

131. The DNA vector of claim 128 or 129, wherein the spacers are at least 20 nucleotides.

132. The DNA vector of claim 128 or 129, wherein the spacers are at least 25 nucleotides.

133. The DNA vector of any one of claims 113-132, wherein the at least one DD-ITR is generated from an AAV ITR, a parvovirus ITR, or a synthetic ITR.

134. The DNA vector of any one of claims 113-133, wherein the DNA vector construct comprises two DD-ITRs.

135. The DNA vector of any one of claims 113-134, wherein the D regions are from different stereotypes than the ITR.

136. The DNA vector of claim 134, wherein each DD-ITR is derived from a different viral serotype.

137. The DNA vector of claim 134, wherein one DD-ITR is derived from an AAV2 ITR, and a second DD-ITR is derived from an AAV5 ITR.

138. The DNA vector of any one of claims 113-137, wherein there is a deletion, substitution and/or insertion in the B and B′ or C and C′ region.

139. The DNA vector of any one of claims 113-138, wherein there is a deletion, substitution and/or insertion in the A and A′ region.

140. The DNA vector of any one of claims 113-139, wherein the DNA vector construct further comprises a partial protelomerase binding site and wherein the covalently closed ends are formed by protelomerase enzyme activity in vitro.

141. The DNA vector of any one of claims 113-140, wherein the DNA vector construct persists within the target cell and results in sustained expression of the predetermined sequence.

142. The DNA vector of any one of claims 113-141, wherein the DNA vector construct can be converted into a concatemeric structure in the cell.

143. The DNA vector of any one of claims 113-142, wherein the sustained expression of the predetermined DNA sequence in the target cell is for a period of time at least at least 2-5 weeks, at least 1-12 months, at least 1-10 years.

144. The DNA vector of any one of claims 142-143, wherein the concatemeric structure persists in the target cell and results in sustained expression of the predetermined sequence.

145. The DNA vector of any one of claims 142-143, wherein the concatemeric structure persists in the target cell extra-chromosomally.

146. The DNA vector of any one of claims 142-143, wherein the concatemeric structure integrates into the target cell chromosome.

147. The DNA vector of any one of claims 113-146 wherein nucleic acid is a therapeutic nucleic acid.

148. A pharmaceutical composition for delivery of a nucleic acid to a target cell comprising the DNA vector of any of claims 44-76 and 113-147 and pharmaceutically acceptable carrier for delivery into a target cell, wherein the target cell is selected from the group consisting of a neural cell, lung cell, retinal cell, epithelial cell, smooth muscle cell, skeletal muscle cell cardiac muscle cell, pancreatic cell, hepatic cell, kidney cell, myocardial cell, bone cell, spleen cell, keratinocyte, fibroblast, endothelial cell, prostate cell, germ cell, progenitor cell, stem cell, cancer cell, and tumor cell.

149. The pharmaceutical composition of claim 148, wherein the composition is administered to the target cell in vivo for treatment of a disease or disorder.

150. The method of claim 28, wherein the host cell has been designed to encode at least a first Tel recombinase under the control of an inducible promoter, wherein said cell comprises an expression vector adapted to produce a bacterial sequence-free vector, said vector comprising an expression cassette, and a nucleic acid of interest are flanked by at least one DD-ITR, and on either side by a target sequence for the Tel recombinase.

151. The method of claim 150, wherein integrated within non-binding regions of the Tel target sequence are target binding sequences for one or more additional recombinases.

152. The method of claim 151, wherein the one or more additional recombinases is selected from the group consisting of pK02 telRL site, the telRL site, the pal site, the loxPsite, the FRT site, phiC31, attP site and the XattP site.

153. A method of producing a linear covalently closed vector containing at least one DD-ITR comprising incubating the host cell of claim 150 under conditions suitable to permit expression of the first recombinase to result in a linear covalently closed vector.

154. A method of producing a circular covalently closed vector containing at least one DD-ITR comprising incubating the host cell of claim 151-152 under conditions suitable to permit expression of a second recombinase to result in circular covalently closed vector.

155. The method of claim 150, wherein the Tel recombinase target site is the phage PY54 Tel 142 base pair target site.

156. The method of claims 150-155 wherein the nucleic acid of interest are flanked on both sides by DD-ITRs.


SEQUENCE LISTING (82 sequences)

SEQ ID NO: 1 - DNA, 22 bp, Unknown
  source: /note=“Description of Unknown Bacteriophage perfect inverted repeat sequence”
  modified_base (1)..(1): a, c, t, g, unknown or other
  modified_base (5)..(6): a, c, t, g, unknown or other
  modified_base (9)..(10): a, c, t, g, unknown or other
  modified_base (13)..(14): a, c, t, g, unknown or other
  modified_base (17)..(18): a, c, t, g, unknown or other
  modified_base (22)..(22): a, c, t, g, unknown or other
ncatnntanncgnntannatgn

SEQ ID NO: 2 - DNA, 22 bp, Unknown
  source: /note=“Description of Unknown Bacteriophage perfect inverted repeat sequence”
ccattatacgcgcgtataatgg

SEQ ID NO: 3 - DNA, 22 bp, Unknown
  source: /note=“Description of Unknown Bacteriophage perfect inverted repeat sequence”
gcatactacgcgcgtagtatgc

SEQ ID NO: 4 - DNA, 22 bp, Unknown
  source: /note=“Description of Unknown Bacteriophage perfect inverted repeat sequence”
ccatactatacgtatagtatgg

SEQ ID NO: 5 - DNA, 22 bp, Unknown
  source: /note=“Description of Unknown Bacteriophage perfect inverted repeat sequence”
gcatactatacgtatagtatgc

SEQ ID NO: 6 - DNA, 14 bp, Unknown
  source: /note=“Description of Unknown Bacteriophage perfect inverted repeat sequence”
attatatatataat

SEQ ID NO: 7 - DNA, 24 bp, Unknown
  source: /note=“Description of Unknown Bacteriophage perfect inverted repeat sequence”
ggcatactatacgtatagtatgcc

SEQ ID NO: 8 - DNA, 42 bp, Unknown
  source: /note=“Description of Unknown Bacteriophage perfect inverted repeat sequence”
acctatttcagcatactacgcgcgtagtatgctgaaataggt

SEQ ID NO: 9 - DNA, 90 bp, Unknown
  source: /note=“Description of Unknown Bacteriophage perfect inverted repeat sequence”
cctatattgggccacctatgtatgcacagttcgcccatactatacgtatagtatgggcgaactgtgcatacataggtggcccaatatagg

SEQ ID NO: 10 - DNA, 56 bp, Artificial Sequence
  source: /note=“Description of Artificial Sequence Synthetic oligonucleotide”
tatcagcacacaattgcccattatacgcgcgtataatggactattgtgtgctgata

SEQ ID NO: 11 - DNA, 42 bp, Artificial Sequence
  source: /note=“Description of Artificial Sequence Synthetic oligonucleotide”
atgcgcgcatccattatacgcgcgtataatggcgataataca

SEQ ID NO: 12 - DNA, 52 bp, Artificial Sequence
  source: /note=“Description of Artificial Sequence Synthetic oligonucleotide”
tagtcacctatttcagcatactacgcgcgtagtatgctgaaataggttactg

SEQ ID NO: 13 - DNA, 90 bp, Artificial Sequence
  source: /note=“Description of Artificial Sequence Synthetic oligonucleotide”
gggatcccgttccatacatacatgtatccatgtggcatactatacgtatagtatgccgatgttacatatggtatcattcgggatcccgtt

SEQ ID NO: 14 - DNA, 38 bp, Artificial Sequence
  source: /note=“Description of Artificial Sequence Synthetic oligonucleotide”
tactaaataaatattatatatataattttttattagta

SEQ ID NO: 15 - protein, 735 aa, Adeno-associated virus [sequence omitted]

SEQ ID NO: 16 - DNA, 21 bp, Artificial Sequence
  source: /note=“Description of Artificial Sequence Synthetic primer”
cgcatattaccywtaacacac

SEQ ID NO: 17 - DNA, 22 bp, Artificial Sequence
  source: /note=“Description of Artificial Sequence Synthetic primer”
gcgtataatggrcwattgtgtg

SEQ ID NO: 18 - DNA, 11 bp, Artificial Sequence
  source: /note=“Description of Artificial Sequence Synthetic primer”
gcgtataatgg

SEQ ID NO: 19 - DNA, 11 bp, Artificial Sequence
  source: /note=“Description of Artificial Sequence Synthetic primer”
ccattatacgc

SEQ ID NO: 20 - DNA, 15 bp, Artificial Sequence
  source: /note=“Description of Artificial Sequence Synthetic primer”
cacacaatwgyccat

SEQ ID NO: 21 - DNA, 15 bp, Artificial Sequence
  source: /note=“Description of Artificial Sequence Synthetic primer”
atggrcwattgtgtg

SEQ ID NO: 22 - DNA, 31 bp, Artificial Sequence
  source: /note=“Description of Artificial Sequence Synthetic primer”
gcggatcctgggttactttaatttgtgtgtt

SEQ ID NO: 23 - DNA, 36 bp, Artificial Sequence
  source: /note=“Description of Artificial Sequence Synthetic primer”
cgctcgagttactccatattttcagtccatgcttgt

SEQ ID NO: 24 - DNA, 34 bp, Artificial Sequence
  source: /note=“Description of Artificial Sequence Synthetic primer”
atcggatcccgatatccagagacttagaaacggg

SEQ ID NO: 25 - DNA, 39 bp, Artificial Sequence
  source: /note=“Description of Artificial Sequence Synthetic primer”
atataaagcttcttttagctgtagtacgtttcccatgcg

SEQ ID NO: 26 - DNA, 30 bp, Artificial Sequence
  source: /note=“Description of Artificial Sequence Synthetic primer”
ggaaattccggtcgctggcgtttctatgac

SEQ ID NO: 27 - DNA, 31 bp, Artificial Sequence
  source: /note=“Description of Artificial Sequence Synthetic primer”
cgctcgagtgaatattagtgcttacagacag

SEQ ID NO: 28 - DNA, 34 bp, Artificial Sequence
  source: /note=“Description of Artificial Sequence Synthetic oligonucleotide”
tccccgcggagctatgaccatgattacgaattgc

SEQ ID NO: 29 - DNA, 33 bp, Artificial Sequence
  source: /note=“Description of Artificial Sequence Synthetic oligonucleotide”
ggactagtccccattcaggctgcgcaactgttg

SEQ ID NO: 30 - DNA, 32 bp, Artificial Sequence
  source: /note=“Description of Artificial Sequence Synthetic oligonucleotide”
gctctagagcaggctgcgcaactgttgggaag

SEQ ID NO: 31 - DNA, 54 bp, Artificial Sequence
  source: /note=“Description of Artificial Sequence Synthetic oligonucleotide”
tatcagcacacaattgcccattatacgcgcgtataatggactattgtgtgctga

SEQ ID NO: 32 - DNA, 42 bp, Artificial Sequence; identical to SEQ ID NO: 8
  source: /note=“Description of Artificial Sequence Synthetic oligonucleotide”

SEQ ID NO: 33 - DNA, 33 bp, Artificial Sequence
  source: /note=“Description of Artificial Sequence Synthetic oligonucleotide”
taacttcgtatagcatacattatacgaagttat

SEQ ID NO: 34 - DNA, 34 bp, Artificial Sequence
  source: /note=“Description of Artificial Sequence Synthetic oligonucleotide”
gaagttcctattctctagaaagtataggaacttc

SEQ ID NO: 35 - DNA, 100 bp, Artificial Sequence
  source: /note=“Description of Artificial Sequence Synthetic polynucleotide”
cccaggtcagaagcggttttcgggagtagtgccccaactggggtaacctttgagttctctcagttgggggcgtagggtcgccgacaygacacaaggggtt

SEQ ID NO: 36 - DNA, 100 bp, Artificial Sequence
  source: /note=“Description of Artificial Sequence Synthetic polynucleotide”
tgatagtgacctgttcgttgcaacacattgatgagcaatgcttttttataatgccaactttgtacaaaaaagctgaacgagaaacgtaaaatgatataaa

SEQ ID NO: 37 - DNA, 430 bp, Artificial Sequence
  source: /note=“Description of Artificial Sequence Synthetic polynucleotide”
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatta

SEQ ID NO: 38 - DNA, 381 bp, Artificial Sequence
  source: /note=“Description of Artificial Sequence Synthetic polynucleotide”
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcgg

SEQ ID NO: 39 - DNA, 329 bp, Artificial Sequence
  source: /note=“Description of Artificial Sequence Synthetic polynucleotide”
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggtttaacactc

SEQ ID NO: 40 - DNA, 329 bp, Artificial Sequence
  source: /note=“Description of Artificial Sequence Synthetic polynucleotide”
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggtttaacactc

SEQ ID NO: 41 - DNA, 39 bp, Artificial Sequence
  source: /note=“Description of Artificial Sequence Synthetic oligonucleotide”
ccattctgttccgcatgattcctctgcggaacagaatgg

SEQ ID NO: 42 - DNA, 13 bp, Artificial Sequence
  source: /note=“Description of Artificial Sequence Synthetic oligonucleotide”
  modified_base (5)..(9): a, c, t, g, unknown or other
ggccnnnnnggcc

SEQ ID NO: 43 - DNA, 25 bp, Artificial Sequence
  source: /note=“Description of Artificial Sequence Synthetic primer”
ggaattcaggaacccctagtgatgg

SEQ ID NO: 44 - DNA, 165 bp, Artificial Sequence
  source: /note=“Description of Artificial Sequence Synthetic polynucleotide”
aggaacccctagtgatggagttggccactccctctctgcgcgctcgctcgctcactgagggcgggcgaccaaaggtcgcccgagcccggccctttgggccgggcccctcagtgagcgagcgagcgcgcagagagggagtggccaactccatcactaggggttcct

SEQ ID NO: 45 - DNA, 198 bp, Artificial Sequence
  source: /note=“Description of Artificial Sequence Synthetic polynucleotide”
tacaaaacctccttgcttgagagtgtggcactctcccccctgtcgcgttcgctcgctcgctggctcgtttgggggggcgacggcccaaagggccgtcgtctggcagctctttgagctgccacccccccaaacgagccagcgagcgagcgaacgcgacaggggggagagtgccacactctcaagcaaggaggttttgta

SEQ ID NO: 46 - DNA, 189 bp, Artificial Sequence
  source: /note=“Description of Artificial Sequence Synthetic polynucleotide”
aggaacccctagtgatggagttggccactccctcccccctgtcgcgttcgcgcgctcgctcgctcactgagggcgggcgaccaaaggtcgcccgagcccggccctttgggccgggcccctcagtgagcgagcgagcgcgcgaacgcgacaggggggagggagtggccaactccatcactaggggttcct

SEQ ID NO: 47 - DNA, 191 bp, Artificial Sequence
  source: /note=“Description of Artificial Sequence Synthetic polynucleotide”
tacaaaacctccttgcttgagagtgtggcactctcccccctgtcgcgttcgcgcgctcgctcgctcactgagggcgggcgaccaaaggtcgcccgagcccggccctttgggccgggcccctcagtgagcgagcgagcgcgcgaacgcgacaggggggagagtgccacactctcaagcaaggaggttttgta

SEQ ID NO: 48 - DNA, 196 bp, Artificial Sequence
  source: /note=“Description of Artificial Sequence Synthetic polynucleotide”
aggaacccctagtgatggagttggccactccctcccccctgtcgcgttcgctcgctcgctggctcgtttgggggggcgacggcccaaagggccgtcgtctggcagctctttgagctgccacccccccaaacgagccagcgagcgagcgaacgcgacaggggggagggagtggccaactccatcactaggggttcct

SEQ ID NO: 49 - DNA, 170 bp, Artificial Sequence
  source: /note=“Description of Artificial Sequence Synthetic polynucleotide”
tacaaaacctccttgcttgagagtgtggcactctctctgcgcgctcgctcgctcactgagggcgggcgaccaaaggtcgcccgacgcccggccctttgggccgggcggccctcagtgagcgagcgagcgcgcagagagagtgccacactctcaagcaaggaggttttgta

SEQ ID NO: 50 - DNA, 164 bp, Artificial Sequence
  source: /note=“Description of Artificial Sequence Synthetic polynucleotide”
aggaacccctagtgatggagtggccctccctctctgcgcgctcgctcgctcactgagggcgggcgaccaaaggtcgcccgacgcccggccctttgggccgggcggccctcagtgagcgagcgagcgcgcagagagggagggccactccatcactaggggttcct

SEQ ID NO: 51 - DNA, 202 bp, Artificial Sequence
  source: /note=“Description of Artificial Sequence Synthetic polynucleotide”
tacaaaacctccttgcttgagagtgttggacactctcccccctgtcgcgttcgctcgctcgctggctcgtttgggggggcgacggcccaaagggccgtcgtctggcagctctttgagctgccacccccccaaacgagccagcgagcgagcgaacgcgacaggggggagagtgtccaacactctcaagcaaggaggttttgta

SEQ ID NO: 52 - DNA, 161 bp, Artificial Sequence
  source: /note=“Description of Artificial Sequence Synthetic polynucleotide”
aggaacccctagtatggagttggccactccattctgcgcgctcgctcgctcactgagggcgggcgaccaaaggtcgcccgagcccggccctttgggccgggcccctcagtgagcgagcgagcgcgcagaatggagtggccaactccatactaggggttcct

SEQ ID NO: 53 - DNA, 202 bp, Artificial Sequence
  source: /note=“Description of Artificial Sequence Synthetic polynucleotide”
tacaaaacctccttgcttggagagtgtggcactctccccccctgtcgcgttcgctcgctcgctggctcgtttgggggggcgacggcccaaagggccgtcgtctggcagctctttgagctgccacccccccaaacgagccagcgagcgagcgaacgcgacagggggggagagtgccacactctccaagcaaggaggttttgta

SEQ ID NO: 54 - DNA, 155 bp, Artificial Sequence
  source: /note=“Description of Artificial Sequence Synthetic polynucleotide”
aggaacccctagtgctggagttggccactcccgcgcgctcgctcgctcactgagggcgggcgaccaaaggtcgcccgagcccggccctttgggccgggcccctcagtgagcgagcgagcgcgcgggagtggccaactccagcactaggggttcct

SEQ ID NO: 55 - DNA, 153 bp, Artificial Sequence
  source: /note=“Description of Artificial Sequence Synthetic polynucleotide”
aggaacccctagtggagttggccactcctctgcgcgctcgctcgctcactgagggcgggcgaccaaaggtcgcccgagcccggccctttgggccgggcccctcagtgagcgagcgagcgcgcagaggagtggccaactccactaggggttcct

SEQ ID NO: 56 - DNA, 182 bp, Artificial Sequence
  source: /note=“Description of Artificial Sequence Synthetic polynucleotide”
aggaacccctagtgatggagttggccactccctccacgcgttctgcgcgctcgctcgctcactgagggcgggcgaccaaaggtcgcccgacgcccggccctttgggccgggcggccctcagtgagcgagcgagcgcgcagaacgcgtggagggagtggccaactccatcactaggggttcct

SEQ ID NO: 57 - DNA, 161 bp, Artificial Sequence
  source: /note=“Description of Artificial Sequence Synthetic polynucleotide”
aggaacccctagtggatggagttggccactccctcctctgcgcgctcgctcgctcactgagggcgggcgaccaaaggtcgcccgagcccggccctttgggccgggcccctcagtgagcgagcgagcgcgcagaggagtggccaactccactaggggttcct

SEQ ID NO: 58 - DNA, 182 bp, Artificial Sequence; identical to SEQ ID NO: 56
  source: /note=“Description of Artificial Sequence Synthetic polynucleotide”

SEQ ID NO: 59 - DNA, 177 bp, Artificial Sequence
  source: /note=“Description of Artificial Sequence Synthetic polynucleotide”
aggaacccctagtcgtgatggagttggccactccctcacgtctgcgcgctcgctcgctcactgagggcgggcgaccaaaggtcgcccgagcccggccctttgggccgggcccctcagtgagcgagcgagcgcgcagacgtgagggagtggccaactccatcacgactaggggttcct

SEQ ID NO: 60 - DNA, 177 bp, Artificial Sequence
  source: /note=“Description of Artificial Sequence Synthetic polynucleotide”
aggaacccctagtgatggagttggccactccctcccccctgtcgcgttcgcgcgctcgctcgctcactgagggcgggcgaccaaaggtcgcccgagcccggccctttgggccgggcccctcagtgagcgagcgagcgcgcagagagggagtggccaactccatcactaggggttcct

SEQ ID NO: 61 - DNA, 173 bp, Artificial Sequence
  source: /note=“Description of Artificial Sequence Synthetic polynucleotide”
tacaaaacctccttgcttgagagtgtggcactcttctgctcgctcgctggctcgtttgggggggcgacggcccaaagggccgtcgtctggcagctctttgagctgccacccccccaaacgagccagcgagcgagcagagagagtgccacactctcaagcaaggaggttttgta

SEQ ID NO: 62 - DNA, 194 bp, Artificial Sequence
  source: /note=“Description of Artificial Sequence Synthetic polynucleotide”
tacaaaacctccttgcttagagtgtggcactctccccctgtcgcgttcgctcgctcgctggctcgtttgggggggcgacggcccaaagggccgtcgtctggcagctctttgagctgccacccccccaaacgagccagcgagcgagcgaacgcgacagggggagagtgccacactctaagcaaggaggttttgta

SEQ ID NO: 63 - DNA, 206 bp, Artificial Sequence
  source: /note=“Description of Artificial Sequence Synthetic polynucleotide”
tacaaaacctccttgcttgagagagtgtggcactctctcccccctgtcgcgttcgctcgctcgctggctcgtttgggggggcgacggcccaaagggccgtcgtctggcagctctttgagctgccacccccccaaacgagccagcgagcgagcgaacgcgacaggggggagagagtgccacactctctcaagcaaggaggttttgta

SEQ ID NO: 64 - DNA, 210 bp, Artificial Sequence
  source: /note=“Description of Artificial Sequence Synthetic polynucleotide”
tacaaaacctccttgcttgagagtgtggcactctacgcgtcccccctgtcgcgttcgctcgctcgctggctcgtttgggggggcgacggcccaaagggccgtcgtctggcagctctttgagctgccacccccccaaacgagccagcgagcgagcgaacgcgacagggggcgcgtggagagtgccacactctcaagcaaggaggttttgta

SEQ ID NO: 65 - DNA, 227 bp, Artificial Sequence
  source: /note=“Description of Artificial Sequence Synthetic polynucleotide”
tacaaaacctccttgcttgagagtgtggcactctcacgcgtagatctagaccccctgtcgcgttcgctcgctcgctggctcgtttgggggggcgacggcccaaagggccgtcgtctggcagctctttgagctgccacccccccaaacgagccagcgagcgagcgaacgcgacagggggtctagatctacggtgagagtgccacactctcaagcaaggaggttttgta

SEQ ID NO: 66 - DNA, 198 bp, Artificial Sequence
  source: /note=“Description of Artificial Sequence Synthetic polynucleotide”
tacaaaacctccttgcttgagagtgtggcactctccccgctcgctcgctcgctcgctcgctggctcgtttgggggggcgacggcccaaagggccgtcgtctggcagctctttgagctgccacccccccaaacgagccagcgagcgagcgagcgagcgagcggggagagtgccacactctcaagcaaggaggttttgta

SEQ ID NO: 67 - DNA, 198 bp, Artificial Sequence
  source: /note=“Description of Artificial Sequence Synthetic polynucleotide”
tacaaaacctccttgcttgagagtgtggcactctccccagctaagatgcagctcgctcgctggctcgtttgggggggcgacggcccaaagggccgtcgtctggcagctctttgagctgccacccccccaaacgagccagcgagcgagctgcatcttagctggggagagtgccacactctcaagcaaggaggttttgta

SEQ ID NO: 68 - DNA, 181 bp, Artificial Sequence
  source: /note=“Description of Artificial Sequence Synthetic polynucleotide”
aggaacccctagtgatggagttggccactccctctctgctcgctcgcgcgctcgctcgctcactgagggcgggcgaccaaaggtcgcccgagcccggccctttgggccgggcccctcagtgagcgagcgagcgcgcgagcgagcagagagggagtggccaactccatcactaggggttcct

SEQ ID NO: 69 - DNA, 198 bp, Artificial Sequence
  source: /note=“Description of Artificial Sequence Synthetic polynucleotide”
tacaaaacctccttgcttgagagtgtggcactctcccccctgtcgcgttcgctcagctaagatgcagtttgggggggcgacggcccaaagggccgtcgtctggcagctctttgagctgccacccccccaaactgcatcttagctgagcgaacgcgacaggggggagagtgccacactctcaagcaaggaggttttgta

SEQ ID NO: 70 - DNA, 180 bp, Artificial Sequence
  source: /note=“Description of Artificial Sequence Synthetic polynucleotide”
aggaacccctagtgatggagttggccactccctctctgctcgctcgcgcgctcaagatgcactgagggcgggcgaccaaaggtcgcccgagcccggccctttgggccgggcccctcagttgcatcttgagcgcgcgagcgagcagagagggagtggccaactccatcactaggggttcct

SEQ ID NO: 71 - DNA, 197 bp, Artificial Sequence
  source: /note=“Description of Artificial Sequence Synthetic polynucleotide”
tacaaaacctccttgcttgagagtgtggcactctcccccctgtcgcgttcgctcgctcgctggctcgtttgggggggcgggcgaccaaaggtcgcccgagcccggccctttgggccgggccccccccaaacgagccagcgagcgagcgaacgcgacaggggggagagtgccacactctcaagcaaggaggttttgta

SEQ ID NO: 72 - DNA, 135 bp, Artificial Sequence
  source: /note=“Description of Artificial Sequence Synthetic polynucleotide”
aggaacccctagtgatggagttggccactccctctctgcgcgctcgctcgctcactgaggcggccctttgggccgcctcagtgagcgagcgagcgcgcagagagggagtggccaactccatcactaggggttcct

SEQ ID NO: 73 - DNA, 153 bp, Artificial Sequence
  source: /note=“Description of Artificial Sequence Synthetic polynucleotide”
aggaacccctagtgatggagttggggtctgcgcgcgctcgctcgctcactgagggcgggcgaccaaaggtcgcccgagcccggccctttgggccgggcccctcagtgagcgagcgagcgcgcgcagaccccaactccatcactaggggttcct

SEQ ID NO: 74 - DNA, 164 bp, Artificial Sequence
  source: /note=“Description of Artificial Sequence Synthetic polynucleotide”
aggaacccctagtgatggagttgggactccctctctgcgcgctcgctcgctcactgagggcgggcgaccaaaggtcgcccgagcccggccctttgggccgggcccctcagtgagcgagcgagcgcgcagagagggagtccccaactccatcactaggggttcct

SEQ ID NO: 75 - DNA, 159 bp, Artificial Sequence
  source: /note=“Description of Artificial Sequence Synthetic polynucleotide”
aggaacccctagtgatggagttggggttaactctgcgcgctcgctcgctcactgagggcgggcgaccaaaggtcgcccgagcccggccctttgggccgggcccctcagtgagcgagcgagcgcgcagagttaaccccaactccatcactaggggttcct

SEQ ID NO: 76 - DNA, 157 bp, Artificial Sequence
  source: /note=“Description of Artificial Sequence Synthetic polynucleotide”
aggaacccctagtgatggagttggtccctctctgcgcgctcgctcgctcactgagggcgggcgaccaaaggtcgcccgagcccggccctttgggccgggcccctcagtgagcgagcgagcgcgcagagagggaccaactccatcactaggggttcct

SEQ ID NO: 77 - DNA, 165 bp, Artificial Sequence
  source: /note=“Description of Artificial Sequence Synthetic polynucleotide”
aggaacccctagtgatggagttggggttaacccctctgcgcgctcgctcgctcactgagggcgggcgaccaaaggtcgcccgagcccggccctttgggccgggcccctcagtgagcgagcgagcgcgcagaggggttaaccccaactccatcactaggggttcct

SEQ ID NO: 78 - DNA, 172 bp, Artificial Sequence
  source: /note=“Description of Artificial Sequence Synthetic polynucleotide”
aggaacccctagtgatggagttggccactccctcttcgctcgctcgctggctcgtttgggggggcgacggcccaaagggccgtcgtctggcagctctttgagctgccacccccccaaacgagccagcgagcgagcgaagagggagtggccaactccatcactaggggttcct

SEQ ID NO: 79 - DNA, 181 bp, Artificial Sequence
  source: /note=“Description of Artificial Sequence Synthetic polynucleotide”
aggaacccctagtgatggagttggggttaaccccactccctctctgcgcgctcgctcgctcactgagggcgggcgaccaaaggtcgcccgagcccggccctttgggccgggcccctcagtgagcgagcgagcgcgcagagagggagtggggttaaccccaactccatcactaggggttcct

SEQ ID NO: 80 - DNA, 56 bp, Artificial Sequence; identical to SEQ ID NO: 10
  source: /note=“Description of Artificial Sequence Synthetic oligonucleotide”

SEQ ID NO: 81 - DNA, 56 bp, Artificial Sequence
  source: /note=“Description of Artificial Sequence Synthetic oligonucleotide”
tatcagcacacaattgcccattatacgcgcgtataatgggcaattgtgtgctgata

SEQ ID NO: 82 - DNA, 56 bp, Artificial Sequence
  source: /note=“Description of Artificial Sequence Synthetic oligonucleotide”
tatcagcacacaatagtccattatacgcgcgtataatggactattgtgtgctgata